(19) United States
(12) Patent Application Publication
(10) Pub. No.: US 2026/0185138 A1
(43) Pub. Date: Jul. 2, 2026

(54) APPARATUS AND METHODS USING COUPLED ENZYME LUMINESCENCE ASSAYS FOR THE DETECTION OF ANALYTES AND BIOMARKERS

(71) Applicant: TETmedical, Inc., FAIR HAVEN, NJ (US)

(72) Inventors: ROY COHEN, CORTLAND, NY (US); DAVID R. FISCHELL, FAIR HAVEN, NJ (US); ALEXANDER J. TRAVIS, ITHACA, NY (US); FURKAT MUKHTAROV, ITHACA, NY (US); JAVIER MAYORGA-KINTANAR, ITHACA, NY (US); PRIYANKA MUKHERJEE, ITHACA, NY (US)

(21) Appl. No.: 19/549,203

(22) Filed: Feb. 25, 2026

Related U.S. Application Data
(63) Continuation-in-part of application No. 18/390,011, filed on Dec. 20, 2023.
(60) Provisional application No. 63/477,700, filed on Dec. 29, 2022.

(51) Int. Cl. C12Q 1/527; G01N 33/552; G01N 33/58

(52) U.S. Cl. CPC C12Q 1/527; G01N 33/552; G01N 33/581; G01N 2333/91215; G01N 2333/988

(57) ABSTRACT

A coupled tethered enzyme luminescence assay for measuring the amount of enzymatic activity of neural specific enolase in a liquid blood sample taken from a patient. The assay has a test well and a positive control well. The test well has a number of components including an inhibitor and a number of first tethered enzyme nanobots formed by tethering pyruvate kinase enzyme to silica nanoparticles, and a number of second enzyme nanobots formed by tethering luciferase molecules to silica nanoparticles for oriented immobilization of the tethered enzymes pyruvate and luciferase. The pyruvate kinase and luciferase enzymes have two differing types of affinity tags. One type of affinity tag facilitates extraction of enzymes from a liquid and another affinity tag for tethering to a silica nanoparticle. The positive control well includes the components of the test well and an added preset amount of enolase enzyme.

### FIELD OF THE INVENTION

The present invention relates to tethered enzyme-based assays for the detection of biomarkers.

### BACKGROUND OF THE INVENTION

There is a great clinical need for improved In-Vitro Diagnostics (IVD) and Point-of-Care (“PoC”) Tests so that accurate diagnoses can be made quickly, enabling appropriate treatment or response as early as possible. A rapid detection system for the diagnosis of neural injury (e.g., stroke, concussion, trauma, aneurism) is especially important, because the treatment options for certain neural injuries such as stroke are extremely time sensitive, with maximal benefits occurring only if treatment can be initiated within the first few hours post-event. In addition, objective/quantitative diagnosis of patients presenting with suspected acute stroke upon arrival at a medical facility is largely limited to computerized axial tomography (i.e., CAT or CT scan), which can typically only accurately identify hemorrhagic strokes (i.e., bleeding in the brain). Such strokes constitute about 15% of strokes. The diagnosis of Acute Ischemic Stroke (AIS) depends predominantly on clinical evaluation based primarily on patient symptoms and clinical signs (e.g., National Institutes of Health Stroke Scale (NIHSS)), and no remarkable findings on the CT scan. Magnetic resonance imaging (MRI), can provide enhanced information, but is not as widely available and usually cannot be performed in a timely fashion, leaving emergency medical providers without a timely means to identify AIS or non-hemorrhagic brain injury. Accurate diagnosis is important, because stroke mimics (non-stroke conditions having similar presentations) account for at least 15% of treatments with tissue plasminogen activator (tPA). Through this over-treatment, these patients are put at risk of hemorrhage, and appropriate diagnostic work-up is delayed for their actual condition. In addition, an average of 17% of strokes are missed, with up to 40% of strokes missed when symptoms are atypical, such as when vertigo and dizziness are the main presenting symptoms. Furthermore, diagnosis of concussion in the field for military applications, or for civilian use such as in athletic settings (field, courtside or rink-side), also depend almost entirely on symptoms because no devices exist that can objectively identify a concussion where symptoms are not clearly evident.

Continuing the example of stroke, current diagnostic methodology relies on neurological expertise and advanced medical imaging techniques (e.g., CT & MRI), which are not widely available, and are time consuming and expensive. Because these limitations are additive to delays in patients reaching the emergency room, only 10-15% of patients suffering from ischemic stroke receive treatment with tissue plasminogen activator (tPA) or similar fibrinolytic drugs within the 4.5-hour effective window (Otite et al., “Ten-Year Trend in Age, Sex, and Racial Disparity in tPA (Alteplase) and Thrombectomy Use Following Stroke in the United States,” Stroke 52:2562-2570 (2021)). In contrast to stroke, today High-Sensitivity Troponin is the standard of care for Acute Coronary Syndromes (ACS) including heart attack. A negative high-sensitivity troponin result provides an accurate means to rule out ACS. Unfortunately, there is no current “Troponin for the brain” that can do for stroke what Troponin does for ACS and heart attack.

All together these issues create a large unmet need for a fast, accurate, highly-sensitive IVD for acute ischemic stroke to provide critical information on brain injury. Such a test would be of particular importance when the CT scan is negative for hemorrhagic stroke. As blood is already drawn for acute testing of patients presenting with stroke systems, an ultra-rapid test (results <15 minutes including blood collection), would not disrupt the workflow for the current Standard Of Care (SOC).

Currently, there are no widely available diagnostics that meet these unmet needs. Several new diagnostic technologies have been proposed including sonography, volumetric impedance phase-shift spectroscopy, and microwave tomography. Various biomarkers have been studied, with most detection approaches utilizing antibody-based capture of biomarker antigens, such as ELISA (Dewey HM and Howells DW (2021) Acute Stroke Biomarkers: Are We There Yet? Front. Neurol. 12:619721. doi: 10.3389/fneur.2021.619721 Dewey HM and Howells DW (2021) Acute Stroke Biomarkers: Are We There Yet? Front. Neurol. 12:619721. doi: 10.3389/fneur.2021.619721). One biomarker that has been studied for decades is neuron-specific enolase (NSE), but “although there appear to be multiple associations of NSE levels with stroke, at this time it does not appear that there is a defined role for serum levels of NSE in the diagnosis or prognosis of acute stroke patients” (Anand and Stead, Cerebrovasc. Dis., 2005, 20:213-219). In part, this is because of problems inherent with antibody-based diagnostic approaches, including variation among antibody affinities, inability of antibodies to differentiate between enzymatically functionally active NSE-FA that is acutely released versus inactive protein NSE-P that is steadily present in the peripheral circulation, and signal: noise challenges that arise from amplification of non-specific binding as well as specific binding. There are commercially available enzyme-based assays for enolase (e.g., Sigma, catalog Number MAK178) that could be used to detect activity via absorbance or fluorescence readouts; however, these are only available for research use and are not practical for direct measurement of NSE activity in fresh blood or blood products. These assays pose numerous barriers to clinical use in general, and particularly with regard to time-sensitive applications, such as the numbers of steps, storage conditions, shelf-life, and need to reconstitute multiple discrete reagents, the technical skills needed to perform these steps, lack of broad dynamic range, lack of standardization, lack of clinical interpretation for a given findings, etc.

Travis and Cohen in U.S. Pat. Nos. 9,547,014, 10,550,415 and 11,549,953 that are incorporated herein by reference and US patent applications 63/005/785/2020/019924, and Ser. No. 16/729,793 (TET Prior Art) describe the techniques by which functional enzymes may be tethered to nanoparticles and other structures allowing increased stability for use in diagnostic and therapeutic applications. Specifically in FIG. 6A-6B of U.S. Pat. No. 9,547,014 (the NSE prior art assay), Travis and Cohen describe the assay to detect Functional Activity (FA) of Neuron Specific Enolase (NSE-FA), an enzyme participating in the glycolysis pathway in neurons. NSE-FA in blood is increased as a result of injury to neurons in the brain. As the NSE was a functional, active enzyme (NSE-FA) before its release, it remains active for a number of hours before it becomes an inactive protein (NSE-P) where it loses its ability to function as an active enzyme. This permits use of the assay shown in FIG. 6A-6B of U.S. Pat. No. 9,547,014 to be able to be used to identify recent (acute) neuronal injury. It should also be noted that the prior art does not provide a description on how to produce a diagnostic assay with long shelf life for NSE-FA or for other enzyme biomarkers, nor does it describe a PoC test embodiment of the NSE-FA assay. The prior art discusses the use of positive and negative controls but does not identify the composition or techniques for producing such controls nor methods for their use in providing a qualitative and/or quantitative measurement of NSE-FA or other enzyme biomarker activity.

Fischell, Travis and Cohen in U.S. Pat. No. 12,306,187 (the '187 patent) describe an IVD strip designed to accept plasma or serum to allow luminescence from positive control, negative control and test wells to produce an optical output that can indicate the presence of NSE as a marker for brain injury from neuronal damage. The limitation of requiring plasma or serum separated from whole blood of a patient typically adds ten minutes to the required time for the assay, for the necessary processing (including centrifuging or coagulation) of the blood sample. While the '187 patent describes point-of-care assays using blood separation paper, it does not provide a description of how one might avoid the delay from obtaining plasma or serum for use in an IVD.

The TET Prior Art also mentions tethering of enzymes using oriented immobilization to be used in the detection of biomarkers but does not describe the process by which this can be accomplished. Such oriented immobilization provides advantages in stability to increase shelf life as well as increased sensitivity in coupled enzyme reactions where a sequence of tethered enzymes work sequentially to produce a measurable signal as described in the TET Prior Art.

Because of the enormity of the clinical need, much attention is focused on developing PoC/at-home diagnostic tests to detect pathology-specific biomarkers. Biomarkers for such tests include proteins, lipids, sugars, nucleic acids, or ions. Blood biomarkers for neural injury have received much attention due to the difficulties regarding timely clinical diagnosis. Currently, over 50 candidate bio-molecules including proteins, metabolites and nucleic acids have been identified and investigated for varied applications in diagnosis, outcome prediction, or treatment of stroke (Jickling and Sharp, “Blood Biomarkers of Ischemic Stroke,” Neurotherapeutics 8(3): 349-60 (2011); Saenger and Christenson, “Stroke Biomarkers: Progress and Challenges for Diagnosis, Prognosis, Differentiation, and Treatment,” Clin. Chem. 56(1): 21-33 (2010); Whiteley et al., “Blood Markers for the Prognosis of Ischemic Stroke: A Systematic Review,” Stroke 40(5): e380-9 (2009); Hasan et al., “Towards the Identification of Blood Biomarkers for Acute Stroke in Humans: A Comprehensive Systematic Review,” Br. J. Clin. Pharmacol. (2012); Glushakova et al., “Biomarkers for acute diagnosis and management of stroke in neurointensive care units,” Brain Circulation, 2:28-47 (2016); Kamtchum-Tatuene and Jickling, “Blood Biomarkers for Stroke Diagnosis and Management,” Neuromolecular Med. 21(4):344-368 (2019); and Bejleri et al., “Diagnostic and Prognostic Circulating MicroRNA in Acute Stroke: A Systematic and Bioinformatic Analysis of Current Evidence,” 23(2):162-182 (2021)). The growing list of potential biomarkers provides a useful resource to guide the development of PoC diagnostic technologies. However, there remains a great need for a rapid, easy-to-use, highly-specific detection system for the diagnosis of neural injury, that has a low Limit of Detection (LOD) for the target analyte, which itself is preferably highly-sensitive for detection of the condition of interest (e.g., stroke).

Several examples of PoC biomarker detection technologies for the diagnosis of various diseases have recently been described. These technologies are divided into 3 major categories including chemical-, immunoassay-or nucleic acid-based detection systems with various signal readout methods such as absorbance, fluorescence, luminescence, electrochemical and colorimetric methods (Chin et al., “Commercialization of Microfluidic Point-of-Care Diagnostic Devices,” Lab Chip (2012)). This list includes Atolyzer.RTM. (Atonomics), Triage.RTM. (Alere), Spinit.RTM. (Biosurfit), and i-STAT.RTM. (i-STAT Corp). However, despite such PoC systems, there remains a great need for increased sensitivity and speed in detecting biomarkers, especially neural injury biomarkers and assays for liver and kidney function. With the potential for liver and kidney damage, patients undergoing chemotherapy or other drug regimens have need for frequent blood tests. This requires travel to a test lab today, which poses logistical barriers such as means and accessibility of transportation, missed employment, etc., as well as inconvenience. New at-home blood sampling devices like the TASSO (Seattle, WA) will allow blood samples to be taken and mailed to a test lab. This too has its pitfalls as such mailing and handling of biologics can degrade the sample and mailing is subject to delays, potentially delaying results for days. Today there are very few at-home blood tests available, with the best-known being for glucose for blood sugar monitoring (e.g., for diabetics), and others for blood typing (e.g., Eldoncard). While at-home/PoC tests for more complex biomarkers like SARS-CoV-2 proteins and/or antibodies are now available, such tests are limited to saliva or nasal swabs.

Beyond neural injury, there are a great many applications also in need of PoC diagnostics. For example, bacteria transferred primarily from the mother can cause significant unsolved dental problems in children resulting in the need for expensive sealant treatments to prevent chronic tooth decay. These bacteria produce a surface expressed enolase enzyme (among others) that if detected in either mother or child could be used to identify early need for appropriate treatment.

PoC tests that can be used at-home or in a doctor's office for monitoring liver and kidney function do not exist and are also needed both for patients suffering from chronic diseases as well as the monitoring of numerous therapies including drug regimens and chemotherapies. Furthermore, ability to test for liver and kidney damage and/or function at-home could also accelerate and improve the safety of drug testing, reducing burdens on trial participants and costs, while increasing the demographic and geographic diversity of participants. An at-home blood test that could utilize an at-home blood collection device (e.g., TASSO, TAP, Mitra) to draw the sample, and generate results that can be transmitted electronically to the patient's doctor would have huge benefit. Achieving luminescence-based enzymatic assays for measurement of liver injury markers ALT and AST directly from fresh blood products has been challenging. Specifically, advanced enzyme-based technologies that utilize hydrogen peroxide (H₂O₂) production to generate luminescence or fluorescence readout have only demonstrated functionality in buffer solutions. References include:
Ecem Saygili, Beyza Orakci, Melisa Koprulu, Alper Demirhan, Esra Ilhan-Ayisigi, Yalin Kilic, Ozlem Yesil-Celiktas, Quantitative determination of H2O2 for detection of alanine aminotransferase using thin film electrodes, Analytical Biochemistry, Volume 591, 2020, 113538, ISSN 0003-2697
Thuy, T. N. T. ; Tseng, T. T.-C. A Micro-Platinum Wire Biosensor for Fast and Selective Detection of Alanine Aminotransferase. Sensors 2016, 16, 767. https://doi.org/10.3390/s16060767
Hsueh C J, Wang J H, Dai L, Liu CC. Determination of alanine aminotransferase with an electrochemical nano ir-C biosensor for the screening of liver diseases. Biosensors (Basel). 2011 Jul 12;1(3):107-17. doi: 10.3390/bios1030107. PMID: 25586923; PMCID: PMC4264364

There is thus a strong need for a PoC device similar to the Lucira™ Covid test now sold by Pfizer that can work with a sample of blood, plasma or serum from a blood collection device; pairing a collection and a PoC test device can provide results from luminescence assays at-home, or in an ambulance or doctor's office.

Today, Covid antigen tests used to detect the presence of SARS Covid-19 are used at-home for detection only and not quantification. As blood testing evolves for at-home tests, there is need for a means to exactly measure biomarker levels that will require exact amounts of body fluids to be placed into a test apparatus. Prior art pipetting tools are sufficient for laboratory use with trained professionals, but for at-home use by untrained people, they are not adequate.

Hemolysis, usually determined via the concentration of hemoglobin in plasma or serum, remains an issue in blood testing as more than a quarter of blood draws for routine biochemistry show hemolysis at interfering levels. Hemolysis interference with plasma-based tests is of particular importance when blood test results are needed urgently, such as when patients present with stroke symptoms. PoC tests that can quantify hemolysis so that samples hemolyzed to a degree that would interfere with test results can be discarded, and/or tests that are designed to overcome the components released by the hemolyzed blood cells causing said interference, would have great clinical advantage.

Luminescence generated from assays having at least one test well and one positive control/reference well with freeze-dried components as described in the TET Prior art may exhibit different reaction speeds. For example, the target analyte pre-seeded and lyophilized in the positive control may take longer to react because it must first be rehydrated by addition of the sample. In contrast, the analyte in the plasma sample being assayed is already in solution. The TET prior art does not address means to account for this potential delay in the analysis of the luminescence from the positive control well(s) in the assay.

Use of freeze drying in the preparation of assay strips requires packaging in a low humidity environment. Mass production of a number of such strips could utilize a holder for a 96 well plate reader, so that a liquid handling robot designed to handle 96 well plates could dispense two or more layers into wells. While Fischell et al in the '187 patent describe a process for assays for brain injury and liver function, it does not describe means to manage the production of hundred or thousands of strips to ensure packaging of strips is efficient and the risk of exposing newly freeze-dried strips to moisture is reduced or eliminated.

In addition to presence in neurons (˜4% of soluble protein), NSE and Non-Neuronal Enolase (NNE) are also present in other cells in the brain including glial cells. These are also subject to damage from both traumatic and atraumatic events including concussion, TBI, hemorrhagic strokes, aneurisms and ischemic events like a stroke or cardiac arrest. Today it is commonplace to send blood samples to a central laboratory for antibody-based protein NSE assays in cases of Cardiac Arrest (CA). These are unfortunately limited in usefulness for managing the cases clinically as the results can take a week or more to be available to clinicians. No current on-location same day IVD or point-of-care NSE assay currently exists. With approximately 100,000 total cardiac arrest survivors per year in the US, occurring both within and outside of hospitals, such an assay would be extremely helpful in assessing the amount of brain damage from CA.

Non-Neuronal Enolase (NNE) is present in other cells in the brain and peripheral nervous system but the prior art for diagnosing brain injury has been focused on NSE; reactions that can enhance sensitivity or specificity by detection/measurement of NNE alone or when interpreted in combination with NSE might also have great clinical utility, such as when evaluating complex trauma cases, particularly with unresponsive patients.

The Travis prior art described the production of enzymes having two affinity tags of a single type being designed to allow tethering of said enzymes to two different kinds of surfaces including silica or other types of nanoparticles. Travis et al, however, does not anticipate the use of production of enzymes for tethering with two different types of affinity tags where a first type is designed to facilitate purification by extraction of enzymes during enzyme production/manufacturing and the second type of tag is designed to produce tethering to a specific surface such as a silica nanoparticle.

For the purposes of this specification, the term “biomarkers” is meant to be inclusive of all analytes, including complex organic compounds, enzymes, simple elements and compounds, other small molecules, sequences of nucleic acids (e.g., DNA, RNA, small RNAs, microRNA, long non-coding RNA, circular RNAs, piwi-interacting RNAs, small nucleolar RNAs, DNA and RNA fragments, etc.), and any other organism (e.g., parasites, fungi, bacteria, viruses, and prions), organic, or inorganic structure that provides important information on patient or environmental condition.

The present invention is directed to overcoming these and other deficiencies in the art, inclusive of applications in human and veterinary medicine, and environmental diagnostics.

### SUMMARY OF THE INVENTION

The present invention is a tethered enzyme platform, system and method for assays to detect important medical biomarkers and analytes using Tethered Enzyme Technology (TET). TET represents a significant advance in medical diagnostics compared to prior art in the form of antibody based In-Vitro Diagnostics (IVD). This platform can also be applied to molecular diagnostics such as currently done via polymerase chain reaction (PCR) or reverse transcription-PCR (RT-PCR). The advances include:
Simplicity with minimal user effort—simply pipette serum, plasma or other liquid samples containing the biomarker into the wells.
Simple quantification—due to the design using test, positive and negative control wells/zones there is a significant reduction in the number of reaction wells needed to obtain the desired specificity and sensitivity. Only 1-4 sample reaction wells and a similarly small number of positive and negative control wells are needed for a specific biomarker compared to 20 or more wells often needed for antibody-based assays.
Speed—due to the minimal steps required and the catalytic nature of enzymes, results are produced in as little as 30 seconds from sample addition.
Customization—TET enables detection of a wide range of biomarkers from different classes including enzymes, enzyme substrates, metabolites, ions, and nucleic acid sequences such as DNA (deoxyribonucleic acids), RNA (ribonucleic acids) and microRNA (non-coding short sequences of RNA), in separate or multiplexed assays with a common readout. Historically, detection techniques for nucleic acid sequences (e.g., PCR) and those for protein biomarkers (e.g., ELISA) have used completely different protocols, devices, and instruments.
Assay stability—tethered enzyme reagents are highly stable compared to enzymes in solution. Lyophilized (freeze-dried) TET reagents have been found to be stable for over a year.
Point-of-care applications—TET-based diagnostics designed as IVDs to be used with plate readers can easily be adapted into PoC embodiments using simple blood filtration papers, taking advantage of TET's combination of sensitivity, specificity, speed, stability and simplicity.
Unique—The TET NSE-Functional Activity Stroke Test (NSE-FAST) assay would be the first-in-class objective measurement for acute brain injury associated with stroke and its PoC version being the only PoC assay for acute brain injury.
Utility—TET assays can be used to detect diverse analytes and biomarkers including enzymes (e.g., alanine aminotransferase (ALT) and aspartate aminotransferase (AST), creatine kinase/creatine phosphokinase (CK/CPK)), and metabolites, ions and other small molecules (e.g., potassium, magnesium, phosphorus/phosphate, calcium, iron, nicotinamide adenine dinucleotide (NAD; NADH is the reduced form), nicotinamide adenine dinucleotide phosphate (NADP; NADPH is the reduced form), creatinine, glucose, and uric acid). Alone or in various combinations, these have clinical utility to monitor liver and kidney function and/or damage to liver, kidneys, and cardiac or other muscles. A PoC device could be used for patients suffering from chronic diseases as well as the monitoring of response to numerous therapies including drug regimens and chemotherapies.

The present invention includes novel embodiments of nanoparticle-tethered enzymes (TET nanobots) that provide a significant increase in coupled enzyme activity and stability compared to untethered enzymes and enzymes tethered via non-specific or chemically-specific but biologically non-oriented immobilization techniques. In embodiments of the present invention, TET nanobots improve substrate access to the enzyme's active site and/or conformational freedom needed for the enzyme to be functionally active. The combination of one or more TET nanobots into a single assay reaction enables the detection of different analytes.

In addition, tethering of more than one enzyme in a detection system utilizing a coupled enzyme reaction pathway produces substantial benefits in the specific activity of the coupled reactions versus when enzymes are in solution or attached to a surface via a non-oriented approach (e.g., Cohen 2015, Mukai, 2013, Mukai, 2017)

The assays of many of the preferred embodiments of the present invention described herein are Coupled Tethered Enzyme Luminescence Assays (CTELA) having a multiplicity of wells or test zones that provide a luminescence/photonic/light output to allow the measurement or detection of one or more biomarkers. CTELA utilize the advantages of TET nanobots.

For the remainder of this specification, the terms “well” or “zone” may both be used to describe any space or volume where a TET-related assay reaction occurs. These include but are not limited to a well in an IVD assay multi-well strip or plate, or a zone on a filter paper, sheet material, or other volume in a PoC or IVD assay device.

The present invention TET assay embodiments provide a significant advance in speed due to the catalytic nature of enzyme function (up to hundreds or thousands of reaction events per enzyme per second), the channeling or proximal diffusion of coupled reaction intermediate products from one tethered enzyme to the next (“substrate channeling”), and the ability to provide oriented immobilization of a thousand or more enzymes on each individual nanoparticle. Thus, with up to millions of reactions per second per nanoparticle and thousands of nanoparticles per reaction well, this amplification of the tethered enzymes in TET assays can provide for high-speed biomarker detection similar or superior to antibody-based detection methods and some other enzyme-based assays that do not use tethered enzymes with oriented immobilization. The reaction efficiency of conversion from substrate to product can also be improved, particularly for coupled enzyme reactions.

The novel embodiments described herein include specific TET assay elements that facilitate the catalytic chain reaction that provides the ability to detect biomarkers at rapid speeds (e.g., less than 5 minutes), from contact of the sample with the TET nanobots. This speed is of particular importance in both IVD and PoC assays for time-critical diagnosis of acute brain injury from stroke, concussion or brain injury during childbirth.

The present invention TET assay embodiments for both IVD and PoC are manufactured using a novel method that includes the following steps:
1. Production of the enzymes to be tethered by introduction of coding genetic material (e.g., DNA or RNA sequences) into a biological entity or expression system that will then produce the needed enzymes with two different types of affinity tag with one type suitable for tethering and the other designed to facilitate extraction from the expression system. The biological entity may be mammalian cells, insect cells, yeast or bacteria. The system may also be a “cell-free expression system” containing elements typically found in one or more of the above entities or completely artificial (e.g., protein printing). One or more follow-on purification steps may be included. Using two different affinity tags allows for higher purity of the enzymes tethered to the nanoparticles in step 2. One preferred embodiment of this method of production is having both a 6-histidine tag (6×His) and a silicon dioxide (SiO₂) tag as the two affinity tags.
2. Tethering of the one or more selected enzymes to nanoparticles to form the nanobots that produce the diagnostic tethered enzyme (chain) reaction in one or more test, positive control or negative control wells, and/or additional select controls such as for sample quality such as presence of interfering substances or conditions (including hemolysis), and/or for additional biomarker specificity, such as for sub-types, isoforms, or other variants of a common base biomarker molecule. Note that for sake of simplicity, we use wells as an example, but wherever used, “wells” should be thought to represent various physical devices with various material compositions, including but not limited to paper strips, paper pads, microfluidic channels, other chambers, surfaces, etc., upon or into which the nanobots can be placed or localized.
3. Use of a multiple step freeze/freeze-drying/drying process and layered introduction of materials into test, positive control, negative control, and/or other control wells or zones. This process prevents untimely/premature activation of the diagnostic wells most important for positive controls, promotes better mixing of the TET reagents with the bio-fluid/sample, and provides for a long shelf life. This process also provides for efficiency in laying down materials in the wells to create the test, negative control, positive control and other control wells. Fast mixing is important to provide rapid separation of the luminescence curves generated from test, negative and positive control wells.
4. Use of a custom IVD strip or a custom PoC structure designed to maximize the efficiency of capture of the output signal (e.g., photons/luminescence) from the wells, improve user convenience and optimize sample addition. Microwell embodiments could range from single wells, to small numbers of wells, to strips or entire plates of wells of varying lengths and widths, all of which could be manufactured to fit within a holder or adaptor, enabling usage in standard plate readers. Embodiments include 96 and 386 well plates.
5. Use of a dispensing system to accurately pipette the well mixtures in layers into the wells for production and/or device quality testing.
6. Use of specific designs using paper filtration, microfluidics, or other separation mechanisms for the PoC assay, in which, for example, blood plasma is separated from

blood cells and cell fragments, and in which the blood product (including plasma or serum) will reach the reaction zones/wells in a timely manner with sufficient concentrations to trigger the TET coupled enzyme reactions.
7. Use of a TET PoC device that includes a detector technology (e.g., photodiodes for detection of luminescence output), and a microcomputer to perform a novel algorithm based on the signal output results of test wells and controls to provide both quantitative and qualitative measurements of biomarker presence.
8. A pre-treatment of blood or blood product samples to allow direct measurement of liver enzymes or other analytes that cannot be detected in blood or its products using luminescence or other desired readouts due to one or more interfering factors. Embodiments of the present invention IVD and PoC use three (or more) types of wells and include a calculation or algorithm for measurement and threshold-based evaluation of biomarker level. For the NSE-FAST this can provide the first objective diagnostic for significant acute brain injury from stroke.

With the future of medicine moving more and more toward telemedicine and the need to provide remote assessment of patient condition, the present invention TET assays will enable point-of-care (PoC) diagnosis not only in the ambulance or doctor's office, but also in the home or at athletic venues, or workplaces including military field deployments. For the purposes of this specification, PoC diagnosis includes use of a diagnostic device or assay without the use of the equipment typically found in central diagnostic laboratories (e.g., Labcorp, Quest, or Eurofins) or in a hospital based diagnostic laboratory. For clarity, the term Point-of-Care as used in this specification can represent embodiments of the present invention that can be used at home and/or at the Point-of-Need (PoN) including in an ambulance, on the sideline of an athletic event, on the battlefield or other location that does not have laboratory test facilities.

The TET prior art describes use of a multiple stage coupled enzyme reaction with the final stage providing a signal indicative of the assay measurement. These signals include color change, electrical outputs, fluorescence, and luminescence. The present invention IVD and PoC embodiments may use any of those signals; however, a preferred embodiment would be the use of luminescence as the significant number of photons produced can be easily measured in embodiments using standard photosensitive assay readers for IVD and inexpensive photodiodes for PoC implementations. Three other important advantages of luminescence include high dynamic range, linearity of the readout, and low background signal coming from biological samples.

In an embodiment, a TET IVD Diagnostic Assay System (TET-IVD) would utilize an assay strip with multiple wells that is insertable into a standard plate reader. A liquid sample (pre-treated or not pre-treated) containing the biomarker would be pipetted into each well; the strip would then be inserted into a plate reader or plate reader-like device where a custom algorithm or set of calculation/algorithms would compare the luminescence, fluorescence, absorbance or color change from the test, negative control and positive control wells to measure the biomarker presence and amount/activity in the sample.

In another embodiment, a TET Point-of-Care Device (TET-PoC) would include electronics attached to photo-detectors (e.g., photodiodes) for the detection of photons/light produced in the test and control wells or zones by an assay, for example, the assay of FIG. 6A-6B of U.S. Pat. No. 9,547,014 where a TET particle utilizes an enzyme that will luminesce such as Luciferase or HRP (Horse Radish Peroxidase). In embodiments, one useful type of photo-detector is a Silicon PhotoMultiplier (SIPM), for example the MicroFC-10035 from Onsemi.

It is important to have reliable calibration methods for PoC readers. An additional aspect of the present invention is to have a separate calibration strip or PoC card having optical sources (e.g., light-emitting diodes (LEDs)), that can be driven to simulate the output of a biomarker assay and/or establish sensitivity calibration of the photo-detectors in any type of optical reader used to measure the luminescence of present invention embodiment assays.

Another embodiment of an internal calibration method could use the existing strip or card activated with an inactive liquid sample that will only activate the positive control/reference well(s)/zone(s). For example, the liquid could be water, artificial plasma or saline solution). Another embodiment of an internal calibration method could use a luminescent protein or chemical reactions that are coupled to upstream enzymes. For example one can use myoglobin (which is not an enzyme) to generate light when in contact with luminol in the presence of H₂O₂ that can be added to the well or produced by an upstream oxidase enzyme.

In an embodiment the TET-PoC has two separable parts, a blood or bio-fluid handling part (disposable cartridge, TET-Card) and an electronic part. The electronic part may be disposable or reusable with the fluid handling part being single use and disposable. It is envisioned that a blood microsampling device (e.g., TASSO® device made by TASSO®, Inc.) could be used to quickly collect a sufficient size blood sample for the TET-PoC.

It is also envisioned that while plasma/serum is mentioned throughout as a fluid for use, the present inventions may also be used with CSF if available.

In a preferred embodiment, the TET-PoC is a disposable unit with both electronics and fluid handling pieces integrated into a single device. For example if the TASSO microsampling device is integrated with a blood filtering mechanism, then plasma or serum can flow into the reaction zones of the TET-PoC where the luminescence produced by one or more TET reactions can be measured by the included photodiodes.

In the preferred embodiment, the TET-PoC is battery powered with electronic circuitry. In other embodiments, the battery powered TET-PoC includes one or more of the following features:
Photodiodes/SIPMs to monitor luminescence produced by any combination of test, positive, negative, and other control wells/zones,
A timer mechanism that disables the device after a pre-set number of days or weeks to ensure the device is used only before its expiration date,
Means to activate the start of the timer mechanism. This can be an actuator, for example, a button, switch or a temperature sensor that activates when the TET-PoC reaches a specified temperature,
Temperature control (e.g., via a heating element and temperature sensor),
GPS chip to provide location/altitude/date data,
Accelerometer or other sensor to ensure correct orientation (to avoid running the test upside down, etc.),
An algorithm implemented in circuitry, a microcomputer or microprocessor to perform calculations for quantifying the amount of a biomarker from the measured luminescence of the zones/wells,
An algorithm implemented in circuitry, a microcomputer or microprocessor based on the luminescence produced by the reactions in the zones/wells to make a threshold-based, yes/no decision,
An algorithm implemented in circuitry, a microcomputer or microprocessor to perform calculations to identify error conditions,
One or more numerical displays, for example a numerical display labeled “AST” and a second numerical display labeled “ALT” both on the TET-PoC to provide liver enzyme data,
One or more visual indicators (e.g., LEDs), that provide a yes/no; low, medium, or high; error or other indication of the assay result or TET-PoC status,
One or more auditory indicators (e.g., sounds produced such as from oscillators, capacitors/resistors, and various buzzers or speakers), that provide “high” or error warning,
Wireless networking of the TET-PoC to local and/or remote smart devices to deliver the test results,
Wired networking of the TET-PoC to local smart devices or computers to electronically deliver the test results to a remote location,
Plug-in or (Re-)charging ability, such as through a USB, USB-C, or magnetic device (e.g., MagSafe, Qi2),
Use of fiber-based (e.g., cellulose, fiber glass or combination) filter micro-channels for wicking blood and/or blood plasma in the TET-PoC from a blood source to the zones/wells containing tethered enzymes and other components for detecting one or more biomarkers. The micro-channels would be 1-10 mm in diameter and in embodiments would include one or more elements selected from the group including:
   1. reservoir paper that wicks whole blood,
   2. a filter mechanism e.g., filter paper that restricts movement of blood cells and cell fragments, but allows plasma/serum to flow on, and
   3. zones/wells comprising one or more assay components including one or more tethered enzymes for detecting a biomarker.

In an embodiment, the TET assay (IVD or PoC) has a multiplicity of test wells/zones including at least one assay test well and at least one negative control well. For example, in a preferred embodiment for the detection of NSE functional activity (NSE-FA), the formulation in the at least one negative control well includes all the components in the test well for detecting NSE except 2-phosphoglycerate (2-PG).

In a preferred embodiment, the TET assay has a multiplicity of test wells/zones including at least one assay test well/zone, at least one negative control well/zone and at least one positive control well/zone. The test, negative control and positive control wells are freeze dried and only become active when they become wet from the presence of the liquid including the potential biomarker, e.g., plasma or serum.

It is also envisioned that an embodiment of the present invention TET-IVD or TET-PoC could have only one zone for test reaction. In a preferred embodiment for the detection of NSE-FA, the formulation of the positive control well(s)/zone(s) includes one or more of the following components to produce a positive reaction when the Tethered Enzyme-based ingredients are activated:
1. 2-PG+Enolase
2. Phosphoenolpyruvate (PEP)
3. Adenosine Triphosphate (ATP)

In a preferred embodiment using Enolase in the positive control well(s)/zone(s), the Enolase is tethered to nanoparticles to improve stability and shelf life.

In an embodiment additional wells/zones may be used in the TET IVD or PoC assays to adjust the final measurement of the biomarker. For example, the TET NSE-FA assay as described by Travis and Cohen in U.S. Pat. No. 9,547,014 may be altered in novel ways to yield additional information on the activity of enzymatically active non-neuronal enolase (NNE) in the sample versus the amount of NSE-FA, based on the isoforms having different characteristics. Use of an inhibitor specific for NSE-FA in an additional well that can be compared to the test well, or use of an inhibitor specific for NNE in the primary test well, or use of an inhibitor of NNE in an additional well that is used to identify the amount of NNE activity by comparison, can be used in various ways with the prior art NSE-FA assay measurement. This can be of most advantage to eliminate measured NNE activity (such as from hemolysis) that could affect the measured assay output. It is also envisioned that a color chart (e.g., quick-reference “Hemolysis Reference Palette” for laboratorians and phlebotomists to determine the hemolysis status of samples, CDC) could be used to identify samples with too much hemolysis causing rejection of that plasma or serum sample.

It is envisioned that to provide an internal test for within-sample reproducibility, 2 or more wells of each type could be used. In a preferred embodiment there is at least one positive control well, at least one negative control well and at least two assay test wells. In a preferred embodiment that minimizes the amount of sample fluid needed for the assay, there is one positive control well, one negative control well and one assay test well. Other embodiments and algorithms to provide consistency are also envisioned such as having 4 of each type of well/zone where the high and low are rejected and the middle two are averaged. Another algorithm/activity calculation would reject results from any well/zone that are considered an outlier compared to the other wells of the same purpose (i.e., test well, positive control or negative control), and calculate the average of the remaining values from that well type. These calculations may apply to the direct luminescence values or to the luminescence curve slopes.

In an embodiment, the IVD or PoC assay includes an algorithm with one or more calculations that can identify significant acute neural injury by comparing the measured luminescence from the test wells with the luminescence from the negative control well and/or the positive control well.

In a preferred embodiment the measurement includes the determination of the slopes of the luminescence outputs of test, positive control and negative control wells. In an embodiment the amount of 2-PG in test wells for NSE-FA is provided in enough quantity that it is not limiting to the reaction such that the limiting component of the reaction is the amount of NSE-FA.

Because a positive control well using the formulation of item 1 above (2-PG and Enolase) will produce a signal that not only depends on the activity of Enolase in the positive control well but the amount of NSE-FA or other Enolase activity introduced in the sample, the activity generated by the positive control alone can be obtained by subtracting from the luminescence of the positive control well, the value of the luminescence from the test well that includes the background luminescence as measured from the negative control well as well as the luminescence generated by NSE in the sample.

In an embodiment, the approximate luminescence generated by the NSE in the sample in the test well can be obtained by subtracting out the luminescence from the negative control well.

In embodiments, it is envisioned that additional mathematical manipulations can be performed to adjust for non-linearity of readouts from one or more wells.

In an embodiment a threshold for detection of an amount of NSE-FA that reflects a pathological state can be set as a percentage of the measured slope of the positive control well luminescence or the difference between the measured slopes of the luminescence of the positive and negative control wells.

In an embodiment a threshold for detection of an amount of NSE-FA that reflects a pathological state can be set as a percentage of the initial reaction slope of the true positive control well luminescence that already subtracts out the negative control well luminescence.

During blood draw, hemolysis may occur that could reduce the effectiveness of the test for NSE-FA. An embodiment of the present invention includes one or more additional wells with a TET-based assay specific to hemolysis that would provide information that can be used to differentiate brain injury from enolase present in blood cells and then released upon hemolysis.

It is also envisioned that embodiments would include one or more wells/zones that can identify levels of hemolysis. This can be used for any of the following:
1. To identify levels of hemolysis that invalidate the assay
2. To identify levels of hemolysis that are usable but require adjustment to the detection/measurement algorithm/calculation of the assay to measure the biomarker.
3. To identify levels of hemolysis within the useable range of the assay.

It is also envisioned that embodiments of the present invention would include changes in the contents of wells/zone to negate interference with the assay from byproducts of hemolysis. For example, one would add, to one or more wells/zones, an adenylate kinase (ADK) inhibitor to reduce non-enolase related conversion of ADP to ATP.

In an embodiment, significant signal from the negative control well or lack of signal from the positive control well will initiate an error condition and subsequent display to the user of the TET-PoC. A similar reading may also result in error conditions for an IVD.

In an embodiment, the TET-PoC includes chemicals in the test well that produce color changes from an assay such as the assay of FIG. 6A-6B of U.S. Pat. No. 9,547,014. Such color changes could be similar to that seen in a COVID-19 antigen test, or pregnancy test.

An embodiment of the TET-PoC would also include a separator to limit movement of blood cells allowing only plasma or serum to flow into the assay wells/zones. This is important as the red blood cells can interfere with the measurement of luminescence. Examples of separators include membranes, filters, chromatography paper magnetic bead separation systems such as those described by Vemulapati in European Patent Application EP3823761A1.

In another embodiment the present invention TET-PoC includes means to extract blood from a human body. For example, if combined with an at-home blood collection device such as the TASSO® blood draw device of U.S. Pat. No. 10,426,390, the integrated, micro-fluidics, mini-centrifuges, and TET-PoC could be used to provide rapid diagnostic test results within a few minutes without need for a phlebotomist, and without need to send the blood sample drawn by a phlebotomist or a device such as the TASSO device to a separate lab. It is also envisioned that some embodiments of means to draw blood would include finger-and heel-pricks, using known devices to perform them.

It is also envisioned that in a preferred embodiment, rather than combine the blood draw device and TET-PoC, a compatible vial that can be removed from the blood draw device would then be inserted into a TET-PoC reader to start the assay. This embodiment also has the advantage of being usable with any blood draw device or means for placing patient blood into a vial.

It is also envisioned that the present invention TET IVD assay may be designed to be inserted into a standard plate reader device. In an embodiment, a TET assay strip of 3 or more wells would be used with a preferred embodiment of 12 wells or 8 wells.

An embodiment of the TET assay may include multiple biomarker assays including separate test wells, negative control well or wells, positive control well or wells, and other control well or wells for each assay. In a preferred embodiment for certain assays, a negative or positive control well may serve for multiple biomarker assays.

In an embodiment of the present invention, a simple to use apparatus and method for placing an exact amount of body fluid (e.g. blood, blood plasma, urine) is strongly needed to allow point-of-care and home use biomarker quantification.

A preferred embodiment of the present invention blood/plasma/serum assay includes additional components in the mixtures placed in the wells or reaction zones to negate the impact of hemolysis on the coupled enzyme reaction. It is also envisioned that such additional components could be placed in a blood collection or other container that would pre-treat the blood sample before or after the blood is centrifuged or converted to plasma or serum.

For the NSE-FAST assay, the main interfering component coming from hemolyzed red blood cells (RBCs) is the enzyme Adenylate Kinase (ADK), which in the forward reaction acts on molecules of ADP to produce ATP. To reduce ADK interference effects coming from hemolysis, we describe in the present invention a reduction in the amount of ADP in the wells and the addition of an ADK inhibitor. These changes to the well composition allow us to obtain meaningful results from samples with hemolysis levels up to 3 on the CDC Hemolysis Reference Palette (within the range of 20 to 50 mg/dL hemoglobin).

A preferred embodiment also includes a hemolysis quantification well (HQW). These wells were designed to generate a signal that is proportional to the levels of hemolysis in real time and without user involvement as opposed to comparing the sample to the CDC hemolysis palette. Options for HQW include 1) a well (PK and Luc) that does not include the ADK inhibitor; 2) a well with luciferase as the only enzyme with no potassium in the mixture; or 3) a well with a peroxide enzyme that generates H₂O₂ and luminol to react with the hemoglobin to generate luminescence.

Another embodiment of the present invention that can quantify the level of hemolysis by measuring the reduction of the total amplitude of the luminescence signal coming from the positive control/reference well/zone.

Another preferred embodiment of the present invention having at least one test well/zone and at least one positive control/reference for quantification of the amount of biomarker in a sample is to utilize an algorithm that compares the data from two or more of the wells/zones during different time windows. For example, one embodiment could compare the slope of the luminescence curve of the test well/zone during a time window from the first 30 seconds to the slope of the luminescence curve of the positive control/high reference during the time window between 30 to 60 seconds.

Another embodiment could compare the slope of the luminescence curve of the test well/zone during a time window from the first 30 seconds to the slope of the luminescence curve of the positive control/high reference at a second analysis period where the start of the second analysis period occurs after a pre-set delay (e.g. more than 10 seconds after the start of the first analysis period).

While Fischell, Travis and Cohen in U.S. Pat. No. 12,306,187 (the '187 patent) describe an IVD strip designed to accept plasma or serum to allow luminescence from positive control, negative control and test wells to produce an optical output that can indicate the presence of brain injury from neuronal death, embodiments of the present invention utilize blood separation techniques for IVDs that can work directly with whole blood, saving significant time and effort in the measurement of biomarkers/analytes as compared with requiring plasma or serum as the IVD input fluid. Specifically, use of an IVD strip with a multiplicity of blood separation paper strips with reaction zones that line up with the location of wells in a 96 well plate could work directly from whole blood but still utilize the functionality of a standard plate reader.

Going beyond descriptions in the '187 patent, the present invention includes apparatus and methods that allow scaling up the manufacturing of the present invention assays, for example, the NSE-FAST, while maintaining high reproducibility and function of the enzymes and coupled activity.

These include:
Making the mixtures for layers in the IVD or PoC with specific apparatus and methods related to:
Manufacturing and tethering the enzymes
Adding reagents
Introducing layers into wells/zones by automated liquid handlers with intermediate freezing steps
Freeze drying of multiple strips at a time
Packaging multiple strips at a time in a controlled environment including introduction of a gas at less than 1 atm pressure to reduce the likelihood of any water vapor that could initiate a reaction in the positive control wells/zones.

It is also envisioned that the present invention NSE Functional Activity Stroke Test (NSE-FAST) would have methods of use for both IVD and PoC versions related to non-stroke disorders in both humans and animals.

While U.S. Pat. No. 12,306,187 describes two approaches to introduction of mixtures into two or three layers, the present invention preferred embodiment for detection of NSE-FA is a different two-layer approach that has:
a base mixture 1 for the negative control/reference wells that receive mixture 1,
mixture 2 having 2-PG added to mixture 1 to produce mixture 2 that is the only layer for the test wells and one of two layers for the positive control/reference well(s) and
mixture 3 that includes a pre-set amount of an enolase enzyme with or without tethering that is the other layer for the positive control/reference well(s)

This preferred embodiment has only a single layer in negative control and test wells and two layers in the positive control wells. This improved manufacturing process reduces well-to-well variability as only the positive control wells have two layers.

It is also envisioned that each lot of manufactured strips during quality testing could have a different sensitivity level creating an adjustment factor for each lot.

While the '187 patent describes other means to produce a positive control without a pre-set amount of the enzyme (or a similar one-e.g., enolase for NSE) being assayed, preferred embodiments using a form of the enzyme being assayed in the positive control/reference wells will reduce the impact of differences in ambient temperature on the measure level of biomarker in the patient sample.

The method of use is the same as that for stroke where a patient's blood, serum or plasma sample is inserted into an assay apparatus having at least one each of test and positive control wells/zones and a measurable signal is generated related to the amount of enzymatically active NSE in the sample. The use of at least one additional negative control well/zone is also envisioned. One additional embodiment would include a well/zone to measure the level of hemolysis in the sample. Another additional embodiment would include a well/zone to measure the amount of non-neuronal enolase in the sample.

An example of present invention method using luminescence for measurement of neuronal injury would include the following steps for a plasma or serum sample:
1. Collect a blood sample from the human or veterinary patient.
2. Use known methods (e.g., centrifugation, coagulation or magnetic beads) to produce a plasma or serum sample from the blood sample
3. Insert a pre-set amount of the plasma/serum sample into the assay having at least two wells/zones so that a portion is input to each well/zone.
4. Measure the amount of luminescence from each well
5. Calculate the amount of active NSE in the sample from the measured luminescence signal.

The embodiments of the present invention using blood separation paper or other means to capture blood cells as described herein would allow step 2 to be skipped in the method described above.

It is also envisioned that the calculation of the amount of active NSE may include adjustments based on any or all of the following:
1. a sensitivity factor determined for each lot of NSE-FAST assays
2. the luminescence curve from a negative control well and/or
3. The amount of hemolysis measured from a hemolysis well/zone
4. Temperature
5. Reader (PoC OR IVD) calibration

While prior art describes the use of fusion proteins, it is envisoned that Coupled Tethered Enzyme Luminescence Assays (CTELA) may function better with various versions of enzyme combinations produced as fusion proteins. These include use of two different enzymes fused (i.e., a “doublezyme”) or three enzymes linked together (a “triplezyme”).

It is also envisioned that embodiments of the present invention IVD and PoC methods of use for assessing brain injury (BI) would be applicable but not limited to the following human uses including traumatic and non-traumatic BI:
Stroke
Traumatic Brain Injury (TBI)
mTBI (Concussion)
Neuro-degenerative diseases (e.g. Alzheimer's)
Epilepsy
Brain tumors e.g. glioblastoma and neuroblastoma
BI resulting from cardiac arrest
BI resulting from anesthesia
Spinal disorders
Infections
Drugs and alcohol related
Asphyxiation
Chronic stress, inflammation
Encephalitis
Toxins including heavy metals
Heat Stroke
Cancers e.g. lung cancer and pancreatic cancer
Dental disease—specifically NNE is present in saliva and can be measured to identify patient at high risk for significant dental decay.

It is envisioned that the biomarkers (e.g. NSE, NNE, ALT, AST etc.) described for the assays in the present invention embodiments for use in human health applications, are also applicable for veterinary use in other animal species.

Thus, an object of the present invention is to provide an in-vitro diagnostic capable of measuring the amount of functionally active NSE (NSE-FA) associated with acute brain injury.

Another object of the present invention is to provide a Tethered Enzyme-based diagnostic for one or more biomarkers including one or more of the following:
1. NSE-FA,
2. ALT, and/or
3. AST

Another object of the present invention is to provide an IVD strip comprising 3 or more wells for use in Tethered Enzyme-based assays where the wells include one or more of the following:
1. Test wells,
2. Positive control wells,
3. Negative control wells, and/or
4. Additional diagnostic wells to provide additional differentiation, e.g., the level of hemolysis in the sample.

Another object of the present invention is to utilize a method for producing one or more of the components of a Tethered Enzyme-based assay, the method including one or more of the following:
1. Nucleic acid-based production of enzymes, which can be followed by purification of the enzyme
2. A process for tethering enzymes to nanoparticles for use in an assay providing specific oriented immobilization that increases the stability and activity of the enzymes to facilitate improved shelf life and shorter detection times than that with non-oriented enzymes, immobilized or not.
3. A process for creating and adding to wells, the mixtures for the assay that prevents premature reaction of the mixtures,
4. A process for freeze drying and packaging the assay to provide a shelf life between hours at room temperature and up to several years in a freezer. A preferred embodiment would have a shelf life of a month at room temperature and a year at normal freezer temperatures.

Still another object of the present invention is to provide for IVD-or PoC-based TET assays, one or more calculations for biomarker measurement based on luminescence from one or more wells including one or more of:
1. Test wells,
2. Positive control wells,
3. Negative control wells, and/or
4. Additional diagnostic wells to provide additional differentiation, e.g. the level of enolase activity introduced from hemolysis in the sample.

Still another object of the present invention is to provide for IVD-or PoC-based TET assays, an algorithm/calculation for biomarker measurement based on the initial reaction rate (slope) of the luminescence data from one or more wells including one or more of:
1. Test wells,
2. Positive control wells,
3. Negative control wells, and/or
4. Additional diagnostic wells to provide additional differentiation, e.g., the level of enolase from hemolysis in the sample.

Still another object of the present invention is to provide an IVD strip with wells shaped to reduce crosstalk and optimize light capture by the photodiode detector or the plate reader. Still another object of the present invention is to provide a formulation of an IVD or PoC luminescence assay to measure liver enzymes ALT or AST directly from plasma or serum using a sample pre-treatment, for example with uric acid and uricase. In a preferred embodiment the pre-treatment materials would be added to or included in a blood collection container. Still another object of the present invention is to provide an IVD embodiment having variable numbers of wells that could all fit within a single adaptor for standard plate readers (e.g., 1, 2, 3, 4, 5, 6, 7, 8, 9, 10, 11, 12, 13, 14, 15, 16, 24, 32, 48, 96, 384 wells). Such an embodiment could allow for readings from a single patient for multiple biomarkers (e.g., different RNA sequences that would be specific for different viral pathogens, or different RNA sequences that would be specific for different variants of a single virus, or various analytes of different kinds that together provide a diagnostic panel that reflects the function of a body system or organ, or pathological condition), or from several patients for one or more biomarkers.

Yet another object of the present invention is to provide a point-of-care device capable of running one or more tethered enzyme-based assays having one or more of the following features:
1. Integrated blood draw microsampling capability of up to 1 ml,
2. Integrated blood/plasma separation,
3. Luminescence detection using photodiodes
4. Inclusion of multiple wells/zones including one or more of the following:
   a. Test wells,
   b. Positive control wells,
   c. Negative control wells, and/or
   d. Additional diagnostic wells to provide additional differentiation or information, e.g., the level of hemolysis in the sample.
5. Interface to allow connection to one or more microsampling vials,
6. Means to ensure appropriate levels of fluid reach each well/zone
7. Staged assembly of assay components during manufacture such as separation of one or more substrates from the primary tethered enzyme mixture, so that premature activation of the reaction is avoided and all required assay components mix upon reconstitution of the freeze-dried reagents,
8. Staged exposure to one or more assay components during flow of the sample such as separation of one or more substrates from the primary tethered enzyme mixture, and/or
9. Calculation-based measurement of luminescence that can be calibrated to provide quantitative and/or qualitative measurement of biomarkers.

Yet another object of the present invention is an embodiment of TET-PoC designed to detect enolase or other surface-expressed enzymes related to the presence of oral bacteria that predispose or are otherwise linked to tooth decay in children.

Yet another object of the present invention is to include components in the assay that help negate the interfering effects of components in a hemolyzed sample on the present invention coupled enzyme reaction.

Yet another object of the present invention is to utilize a well or zone to quantify the level of hemolysis in the sample for a CTELA.

Yet another object of the present invention is to use a portion of the luminescence signal for the positive control/reference that is produced at a different time than that used for the test well/zone. A preferred embodiment has the start of the portion of the signal used for the positive control/reference delayed by more than 10 seconds from that portion used for the test well/zone.

Yet another object of the present invention is to provide a system and method for producing assay IVD test strips or PoC cards in a way that reduces strip exposure to humidity after freeze drying during packaging. This includes using introduction of an inert gas such as nitrogen or argon with a preferred embodiment to have the package sealed with the gas at less than 1 atm pressure

Yet another object of the present invention is to provide a method for assessing conditions other than stroke, concussion or TBI such as assessing the level of brain damage in cardiac arrest patients.

Yet another object of the present invention is to provide a method for assessing neuronal injury in non-human animals of various species and resulting from multiple disorders.

Yet another object of the present invention is to provide an embodiment of the present invention assay with only the positive control/reference well/zone including more than one layer.

Yet another object of the present invention is to provide an embodiment of the present invention assay comprising positive control, negative control and test wells where the positive control comprises two layers with the second layer including a pre-set amount of the biomarker being assayed and all the wells including an ADK inhibitor to reduce the impact of hemolysis, with one or more of the enzymes having two different types of affinity tags one of which is used in the assay to tether the enzyme to a nanoparticle.

Alternate embodiments of the present invention would substitute for the light emission stage of the preferred embodiment to one that results in a change of color, light absorbance, or electrical conductance or resistance.

It is envisioned that the terms positive control and negative control may also represent high and low reference respectively. Together with a test well or zone, these three provide information on both test validity and quantification of the amount of biomarker in the sample.

While the present invention descriptions describe assays for Neuron Specific Enolase, these embodiments are equally appropriate and applicable assays for any active enolase enzyme including isoforms of Non-Neuronal Enolase (NNE).

These and other objects and advantages of this invention will become obvious to a person of ordinary skill in this art upon reading of the detailed description of this invention including the associated drawings as presented herein.

### BRIEF DESCRIPTION OF THE DRAWINGS

FIG. 1 is a block diagram showing an embodiment of the steps in a three stage TET assay, such as for detection of a biomarker that itself has catalytic enzyme activity.

FIG. 2 is a table showing an embodiment of the lists of well components for TET assays for NSE-FA, and liver function enzymes ALT and AST.

FIG. 3A is a diagram showing an embodiment of the test well components for the assay for NSE-FA

FIG. 3B is a diagram showing an embodiment of the process that occurs when a liquid sample containing enzymatically active NSE is inserted into the present invention reaction test well or zone.

FIG. 3C is a block diagram of an embodiment of the method using tethered enzymes to measure liver enzymes directly from plasma or serum.

FIG. 4A is a schematic view of an embodiment of the TET custom IVD strip designed for placement into the holder for a standard plate reader device.

FIG. 4B is a cross-sectional view of an embodiment of the shape of the wells in the strip of FIG. 4A showing up to the three layers of components in the wells.

FIG. 4C is a cross-sectional view of a preferred embodiment showing the shape of three wells in a strip with up to two layers of components in the wells.

FIG. 4D s a schematic view of an embodiment of the TET 8 well assay strip designed for placement into the 8×12 holder for a 96 well plate reader.

FIG. 4E is a cross-sectional view of an embodiment of the wells in the strip of FIG. 4D showing one layer in the test and negative control wells and two layers in the positive control well.

FIG. 5 is a block diagram of an embodiment of the enzyme production process using bacteria to produce enzymes designed for tethering.

FIG. 6 is a block diagram of an embodiment of the enzyme tethering process.

FIG. 7 is a block diagram of an example of a preferred embodiment of the production process for the assay strips of FIG. 4C, including the steps for inserting the materials that are placed into the wells/zones for the present invention TET coupled enzyme assay of FIG. 1.

FIG. 7′ is a block diagram of an example of another preferred embodiment of the production process for the assay strips of FIG. 4D, including the steps for inserting the materials that are placed into the wells/zones for the present invention TET coupled enzyme assay of FIG. 1.

FIG. 8 is a top view of a preferred embodiment of a production module being a standard 96-well, 8-strip holder with eight empty 12-well strips.

FIG. 9A is a top view of the production module with holder and with the eight strips after a first layer has been deposited into the test and negative control wells.

FIG. 9B is a top view of the production module with the eight strips after completion of sub-process 1 of FIG. 7.

FIG. 9B′ is a top view of the production module with eight strips as it would be after step 2-2 of sub-process 2 of FIG. 7.

FIG. 9C is a top view of the production module with eight strips on the freezer block as it would be after completed step 3-1 of sub-process 3 of FIG. 7.

FIG. 9D is a top view of the configuration of the completed production module.

FIG. 9E is a top view of a preferred embodiment of a completed production module having six 8-well strips.

FIG. 10A is a schematic view of the freezer block with depression placed on top of an insulating pad with pedestals to limit the heat flow from the insulating pad to the freezer block so it will remain at a cold temperature during sub-process 3 of FIG. 7.

FIG. 10B is a schematic view of the Freezer block with a handle inserted into slots in the freezer block.

FIG. 11A is a graph showing an example of the luminescence data from a 12-well strip (e.g., the output of an NSE-FA diagnostic strip) following insertion of a patient plasma sample into each well.

FIG. 11B is a graph showing one embodiment of the data used to measure luminescence data from a coupled enzyme reaction.

FIG. 11C is a graph showing an alternate algorithmic embodiment to measure luminescence data from a coupled enzyme reaction.

FIG. 11D is a graph showing a preferred embodiment of the present invention algorithm where the data analyzed for the positive control/reference is from a period different from that for measuring the test well/zone.

FIG. 12A is a top view showing the blood separation paper strip set with negative control strip, test strip and positive control strip as they might be configured before being used in a Point of Care (PoC) diagnostic assay.

FIG. 12B is a top view showing the strips at a time of several seconds after a patient's blood has been placed onto each of the three blood separation paper strips.

FIG. 12C is a top view showing the assay strips as the plasma has now filled the reaction zones and is filling the flow extension zones of the blood separation paper strips.

FIG. 12D is a top view showing an alternate embodiment of the lateral flow blood separation paper strip set.

FIG. 12E is a top view showing the blood separation paper strip set for both liver enzymes ALT and AST as they would be configured before being used in a PoC diagnostic assay.

FIG. 12F is a top view showing another preferred embodiment of the blood separation strip set.

FIG. 13A is a schematic view showing an embodiment of a TET assay card configured for insertion into a photodiode-based reader.

FIG. 13B is a top view showing an embodiment of a TET diagnostic PoC card layout where the fluid input zone is centrally located with 5 blood separation strips leading to five reaction zones.

FIG. 14 is a schematic view of an embodiment of a TET coupled enzyme assay test module where a blood sample volume is deposited into an upper cylinder with strip holder and 4 blood separation paper strips.

FIG. 15 is a schematic view of an embodiment of a two-piece TASSO/TET assay system with a photodiode luminescence reader (PLR) and the TASSO blood collection device and the normal collection vial replaced by an embodiment of the TET coupled enzyme assay test module similar to the test module of FIG. 14 but with the addition of the alignment key.

FIG. 16 is a schematic view of an integrated and fully disposable point-of-care TET coupled enzyme assay system including a TASSO blood collection device.

FIG. 17 is a block diagram of an embodiment of the electronics module that has features that would be incorporated into either or both electronic module embodiments of FIG. 15 and FIG. 16.

FIG. 18 is a schematic view showing a preferred embodiment of a point of care blood collection device such as the TASSO with body, blood collection activator and microtainer shown with collected blood and bottom surface suitable for needle penetration.

FIG. 19 is a schematic view showing a preferred embodiment of a disposable NSE functional activity stroke test (NSE-FAST) assay.

FIG. 20A is a top view showing an embodiment of the present invention assay comprising 8 connected lanes, each having lateral flow blood separation strips sized and positioned to fit within a standard plate reader or other photonic luminescence reader that can function directly from whole blood.

FIG. 20B is a top view showing an embodiment of the present invention assay that can function directly from whole blood where blood has been deposited and is flowing to the left of each blood separation lane as cells are captured and plasma continues to flow toward the reaction zone.

FIG. 20C is a top view showing an embodiment of the present invention assay that can function directly from whole blood where blood has been deposited and is flowing to the left of each blood separation lane as cells are captured and plasma has reached the reaction zone producing luminescence.

FIG. 20D is a top drawing showing three of the present invention assays with the 8 connected lanes of FIG. 20A placed in a 96-well holder that can be inserted into a standard plate reader.

FIG. 21 is a top view of an alternate embodiment of the present invention with 8 blood separation lanes that can provide 16 wells or reaction zones of IVD measurement from 8 centrally located input zones where blood is deposited.

FIG. 22 is a schematic view of an 8-well assay card designed for point-of-care use.

FIG. 23 is a schematic view of a portable point-of-care reader for the 8-well assay card of FIG. 22.

FIG. 24 is a bottom view of the electronics package of the portable point of care reader of FIG. 23.

FIG. 25 is a cross-sectional view of the electronics package of FIG. 24 at 2500-2500.

### DETAILED DESCRIPTION OF THE DRAWINGS

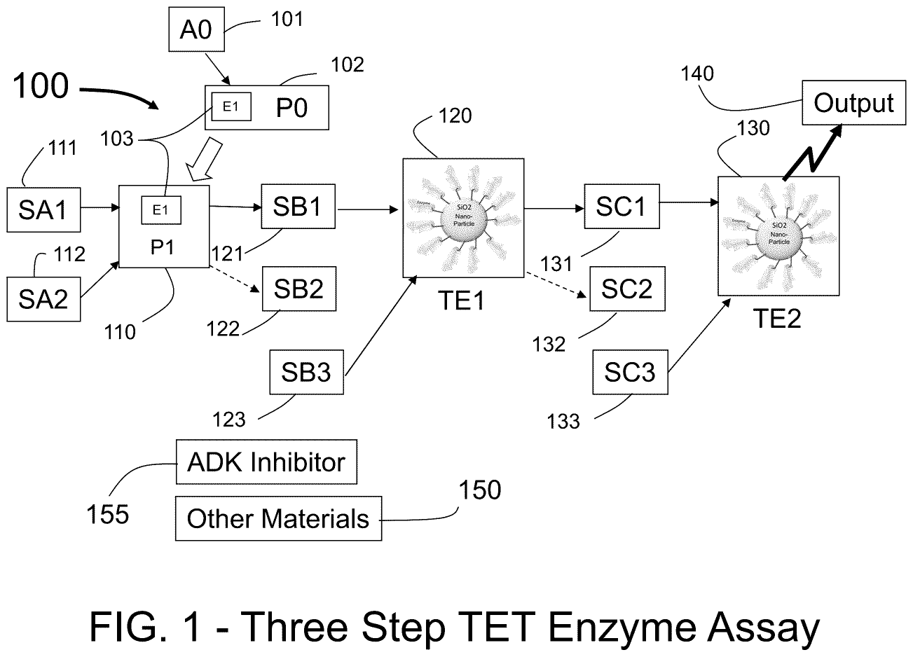

FIG. 1 is a block diagram showing an embodiment of the components in the test reaction well for a three-step TET coupled enzyme assay 100. Element P1 110 that may contain an enzyme biomarker E1 103 is a sample of body fluid (e.g., saliva, urine, mucus, blood or plasma/serum) being assayed, the sample 110 being inserted or flowing into a test well or test zone. In an alternate embodiment important for certain assays, for example assays for the liver enzymes ALT and AST, an additive A0 101 is combined with a precursor sample P0 102 to produce the sample P1 110. For assays that do not require a pre-treatment with A0 101, P0 102 is the same as P1 110.

The test well or zone would typically include the following components at the time the enzyme E1 103 is placed in the well or flows into the zone:
SA1 111 and SA2 112 being the one or more substrates/co-factors/reagents on which the enzyme E1 103 acts to produce the one or more reaction products SB1 121 and/or SB2 122, with at least one that will serve as the substrate taken up by the first tethered enzyme nanobot TE1 120. Note that throughout this text and figures, use of the term “substrate” is meant to include any co-factors (inorganic or organic) or other reagents needed for the activity of the relevant enzyme, whether the enzyme is tethered as part of the TET diagnostic system.
TE1 120, the first tethered enzyme nanobot that acts on the output substrate SB1 121 from E1 103 in conjunction with the substrate SB3 123 that is already in the test well or zone to produce the one or more reaction products SC1 131 and/or SC2 132 that can serve as substrates for the second tethered enzyme nanobot TE2 130.
TE2 130, is the second tethered enzyme nanobot that acts on SC1 131 in conjunction with the substrate SC3 133 already in the well or zone to produce the output signal 140. Examples of output signals include light emission, color changes or electrical signals.
Other materials 150 including buffers and cryo-protectants. For example, Mg2+, KCl, Ca2+, HEPES, dextran, and/or sorbitol.

While FIG. 1 shows a coupled enzyme assay 100 with two tethered enzymes TE1 120 and TE2 130, it is envisioned that embodiments with only one or with three or more sequential coupled enzymes, each producing at least one needed substrate as input for the next in the sequence can be used, with the last tethered enzyme providing an output similar to the output 140 of TE2 130 shown here.

Preferred embodiments of the present invention assay 100 in addition to the other materials 150 listed above, may include the presence of an ADK inhibitor 155 to reduce the impact of hemolysis in the patient sample E1 103.

Examples of ADK inhibitors 155 that may be used in embodiments include but are not limited to:
P1,P5-di(adenosine 5′)pentaphosphate (Ap5A)
Actinomycin D
Diadenosine tetraphosphate (Ap4A)
Diadenosine triphosphate (Ap3A)

In a preferred embodiment the ADK inhibitor 155 is included in the assay wells/zones during manufacturing of the assay.

In another embodiment the ADK inhibitor 155 is included as an additional pre-treatment element A0 101 for the NSE-FA assay.

Such an ADK inhibitor 155 may be placed in (e.g. a prepared system with the ADK inhibitor in liquid or freeze dried format) or added to any of the following:
The blood collection vial,
An intermediary vial into which the blood, plasma or serum is transferred,
The assay wells directly before or after the serum or plasma is pipetted in.

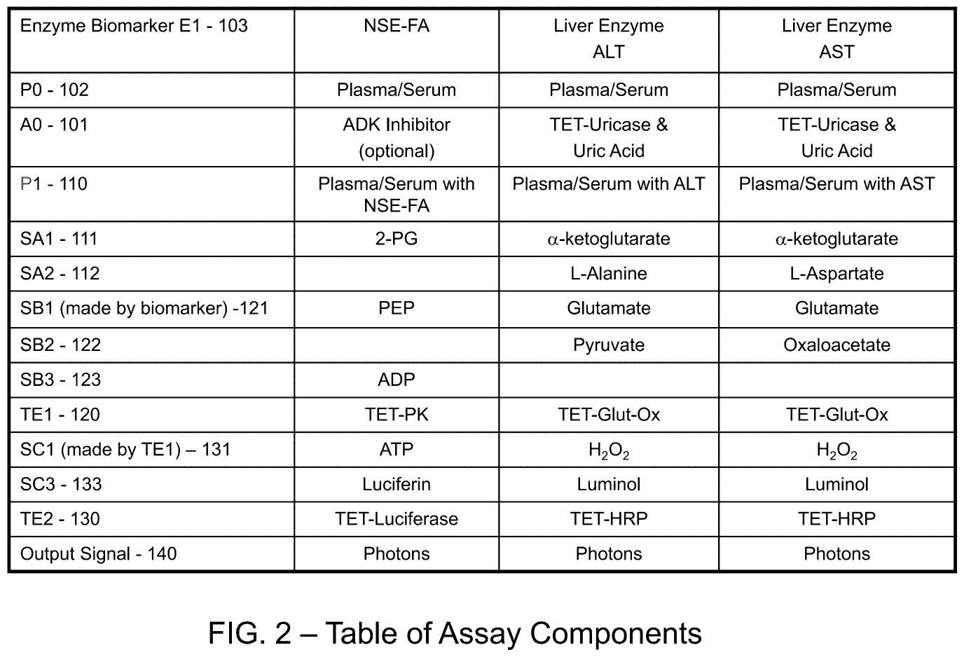

FIG. 2 is a table showing an embodiment of the lists of reaction well/zone components for TET-IVD or TET-PoC luminescence assays for the enzymes NSE-FA and liver enzymes ALT and AST. In a preferred embodiment of this assay formulation for NSE-FA and liver enzymes, P0 102 and P1 110 are plasma or serum produced from a patient's blood.

The pre-treatment element A0 101 for the liver enzyme assays is a combination of tethered uricase (TET-Uricase) plus uric acid that in a preferred embodiment is frozen or freeze dried either in a tablet, powder or onto the inside surface of a vial used to contain the plasma or serum. When thawed or exposed to liquid the uric acid will act as a substrate to the TET-Uricase to produce Hydrogen Peroxide H₂0₂. This pre-treatment step is an important aspect of the present invention as the H₂0₂ produced eliminates anti-oxidants from the sample P0 102 before it is added to the well or zone becoming the sample P1, 110. Specifically, without this step, any anti-oxidants naturally-occurring within the sample such as ascorbic acid, uric acid, and glutathione, would otherwise diminish the enzymatically produced H₂0₂ as input for the final stage of the coupled reaction produced by TE2 130.

It is envisioned that other embodiments of the pre-treatment element A0 101 may include Glutathione s-transferase (GST), superoxide dismutase (SOD), and/or enzymes that are oxidases that following interaction with their subtrates create peroxides including hydrogen peroxide. These include:
Uricase without uric acid
Ascorbate oxidase with or without ascorbic acid, and
Glucose oxidase with or without glucose

The initial substrates SA1 111 and SA2 112 on which the enzyme being detected works are 2-PG for NSE-FA and the combination of α-ketoglutarate and L-Alanine for ALT and α-ketoglutarate and L-Aspartate for AST. SB2 122 represents additional outputs of E1 103 that are not used in subsequent reactions; SB2 122 of the reaction of ALT is Pyruvate and of AST is Oxaloacetate.

The first tethered enzyme TE1 120 is Pyruvate Kinase (PK) for NSE-FA and Glutamate Oxidase (Glut-Ox) for ALT and AST. An additional input pre-seeded in the well or zone for use with TE1 120 being Pyruvate Kinase is ADP as the substrate SB3 123. The additional input substrates SB3-123 required for TE1 120 being glutamate oxidase are oxygen and water supplied by the patient sample. The output of the first Tethered enzyme SC1 131 is ATP for the NSE-FA assay and Hydrogen Peroxide (H₂O₂) for ALT and AST.

The second Tethered enzyme TE2 130 Is Luciferase for NSE-FA and Horse Radish Peroxidase for ALT and AST. The substrate in the mixture SC3 133 that is worked on by the second tethered enzyme TE2 is Luciferin for NSE-FA and Luminol for ALT and AST.

All three assays produce photons through luminescence as the output signal 140 from the second tethered enzyme TE2 130 being luciferase for NSE-FA and Horse Radish Peroxidase (HRP) for ALT and AST.

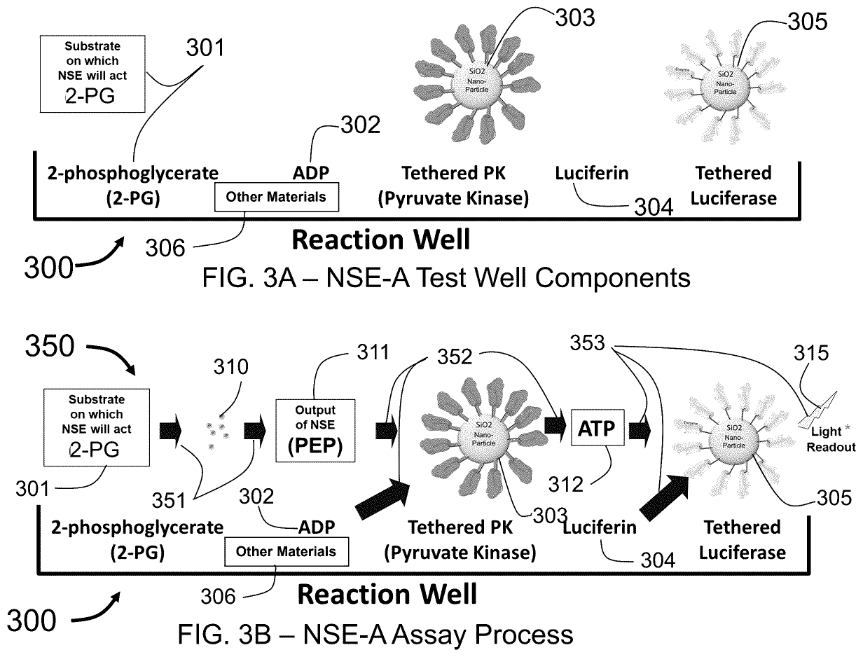

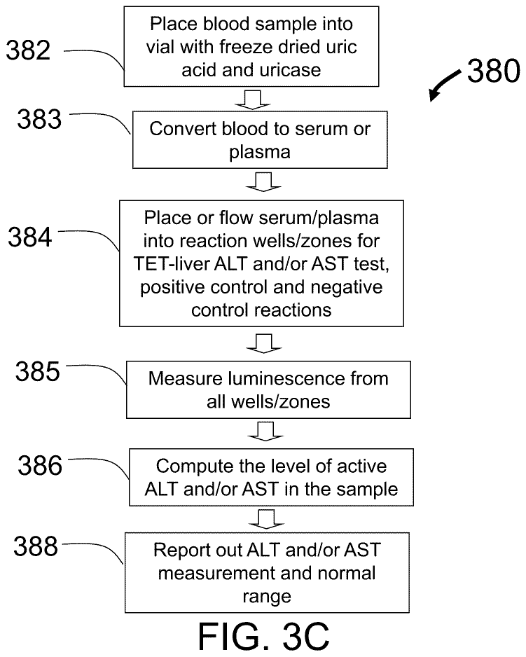

FIG. 3A is a diagram showing an embodiment of the well/zone 300 components for the three stage TET assay for functional activity of NSE (NSE-FA). These are the substrates 2-phosphoglycerate (2-PG) 301, Adenosine Diphosphate (ADP) 302, the first tethered enzyme Pyruvate Kinase (TET-PK) 303, Luciferin 304, the 2^{nd} tethered enzyme Tethered Luciferase (TET-Luciferase) 305 and other materials 306.

FIG. 3B is a diagram showing an embodiment of the process 350 that occurs when active NSE-FA 310 is introduced into the reaction well 300. The sequential steps are:
Step 351. The functionally active NSE placed in the well or flowing into a zone (NSE-FA) 310 will take up the substrate 2-PG 301 to produce Phosphoenolpyruvate (PEP) 311. The PEP 311 is released into the well/zone where the step 351 will continue so long as there is 2-PG 301 in the well.
Step 352. The first tethered enzyme TET-PK 303 will take up the PEP 311 from step 351 and ADP 302 pre-seeded in the well/zone to produce Adenosine Triphosphate (ATP) 312. The ATP 312 is released into the well/zone where step 352 will continue for the needed measurable time period as long as there is PEP 311 from step 351 and ADP 302 in the well.
Step 353. The second tethered enzyme TET-Luciferase 305 will take up the ATP 312 and Luciferin 304 pre-seeded in the well to produce luminescence (a light output signal) 315 with close to one photon produced per ATP 312 molecule produced. Step 353 will continue for the needed measurable time period as long as there is ATP 312 from step 352 and luciferin 302 in the well.

With each molecule of the NSE biomarker capable of performing at least hundreds of reactions per second, and each nanobot having hundreds to thousands of enzymes tethered to each silica (SiO₂) nanoparticle, with an immobilization and enzyme orientation designed to optimize enzyme stability and activity in coupled enzyme reactions, and each of these hundreds to thousands of enzymes producing the reactions of steps 352 or 353, the overall signal production is extremely rapid. The total amount of signal is primarily limited by the amount of activity of the NSE-FA in the sample because no other substrates, co-factors or enzymes are present in limiting quantities for the desired assay time period that is typically less than 10 minutes and ideally less than 3 minutes. The embodiments of the present invention described in the remaining figures also allow excellent dynamic range of detection of the NSE-FA and the ability to directly assay the activity from plasma. While the preferred embodiment would use silica nanoparticles, it is envisioned that other nanoparticles would work so long as the nanoparticle material is transparent, translucent or reflective. For example, such nanoparticles would include:

Polycarbonate, acrylics such as Lucite, diamond, ceramics, other polymers, silver, gold and platinum. While nanoparticles typically range in size from 1 nm to 500 nm, it is also envisioned that other sized or non spherical particles may be used.

While the preferred embodiment uses nanoparticles that are approximately spherical, it is envisioned that embodiments using other shapes are possible including the following shapes:
a. Cylindrical including nanowires,
b. Mesoporous,
c. Plates,
d. Oblate spheroids, and
e. Other heterogeneous shapes
   including beads, solid rods or other surfaces, or shapes extending from a surface that could be used to immobilize and stabilize the tethered enzymes.

FIG. 3C is a block diagram of an embodiment of the present invention method 380 to measure liver enzymes ALT and/or AST directly from plasma or serum using embodiments of the present invention tethered enzyme technology. The method 380 begins with step 382 where a blood sample is placed into a vial or container into which freeze dried uric acid and uricase have been added. In a preferred embodiment, the vial has the freeze-dried materials attached to a portion of the inner surface of the vial that is sealed and packaged in preparation for future use. In an alternate embodiment, powder or a tablet containing uric acid and uricase can be separately added to the vial. In other embodiments, the vial would be a vacutainer. In another preferred embodiment, the uricase is in the form of a tethered enzyme where the tethering may be either to the surface of the vial or to another surface such as that of a silica nanoparticle.

Next in step 383 the blood is converted to serum or plasma. In a preferred embodiment it is envisioned that step 383 could be done first where the blood is converted to serum or plasma before it is placed in the vial with uric acid and uricase.

Next in step 384, the serum or plasma from steps 382 and 383 is placed or flowed into one or more test reaction wells or zones having the materials described in FIGS. 1 and 2 for the liver enzymes ALT and/or AST. A portion of the plasma or serum would also be placed in one or more negative control wells or zones where one or more of the initial substrates SA1 111 and SA2 112 shown in FIG. 2 are absent. Finally, a third portion of the plasma or serum would be placed or flowed into one or more positive control wells or zones with the same components as the test wells/zones but also including a pre-seeded amount of the enzyme biomarker (e.g., ALT and/or AST).

While the preferred embodiment uses positive and negative control reactions, it is also envisioned that embodiments of low range and high range reactions could be used.

Next in step 385, the luminescence is measured from all the wells/zones, and

[symbol]

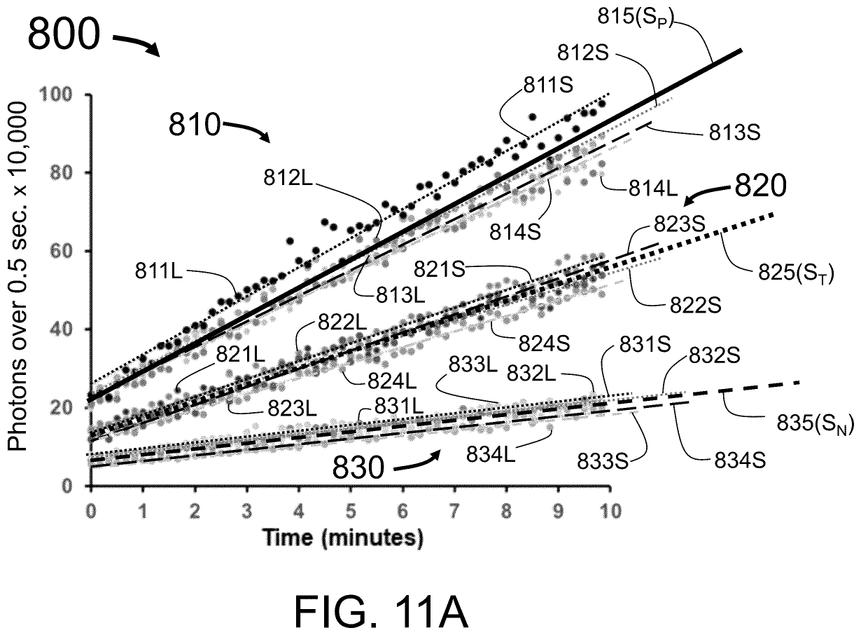

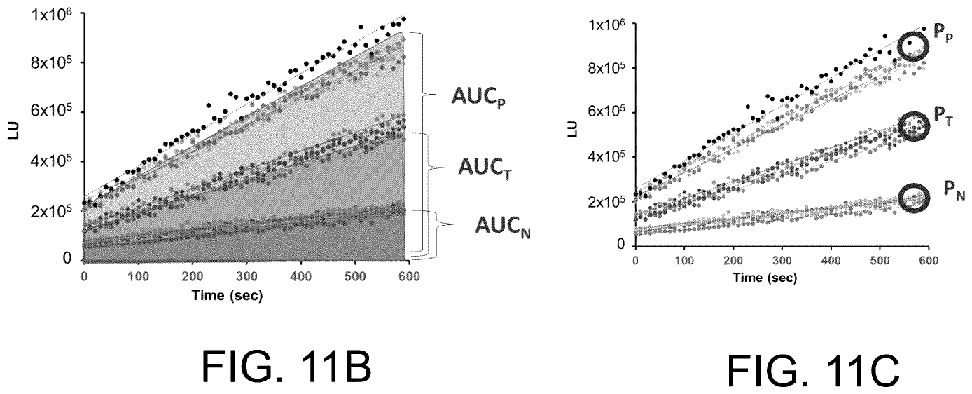

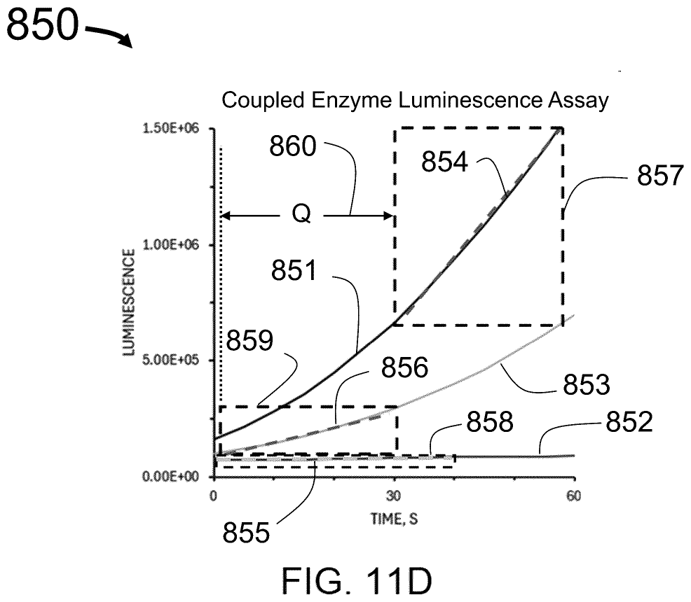

In step 386 the level of active ALT and/or AST is computed based on the luminescence measured from the test, positive and negative control wells/zones (e.g., as described with FIGS. 11A, 11B and 11C).

Finally in step 388, the quantitative measurement of ALT and/or AST activity is reported out and may include a display of the normal range of measurement of each.

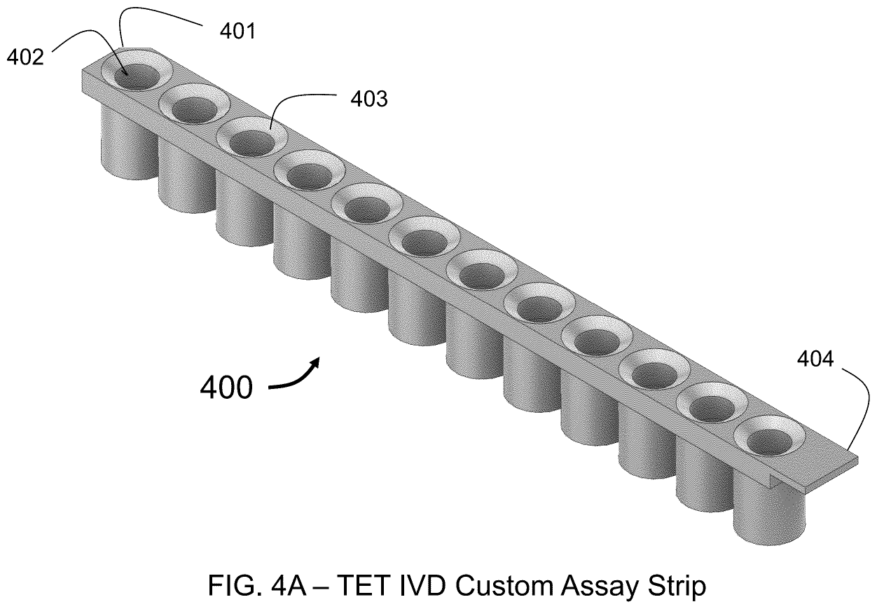

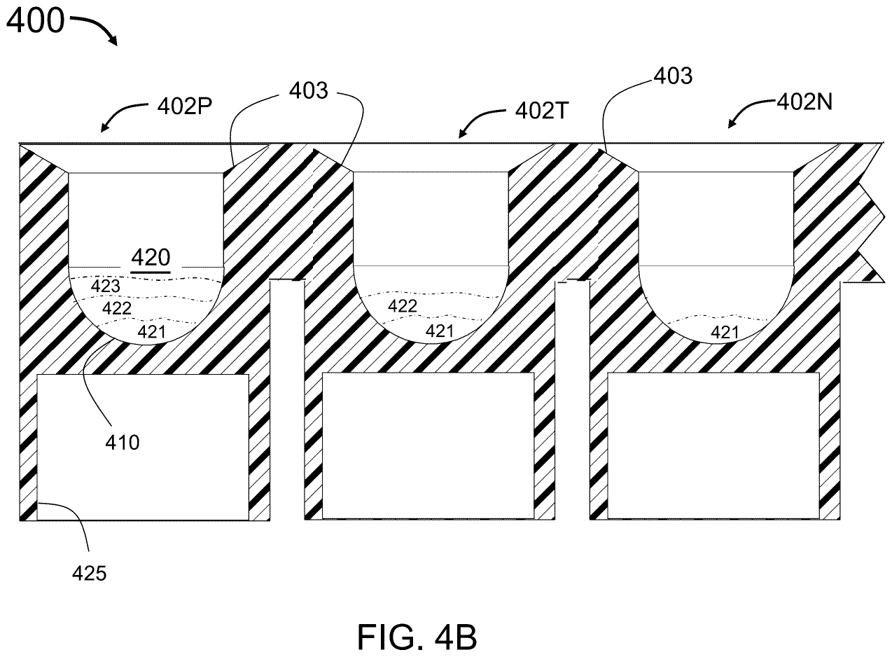

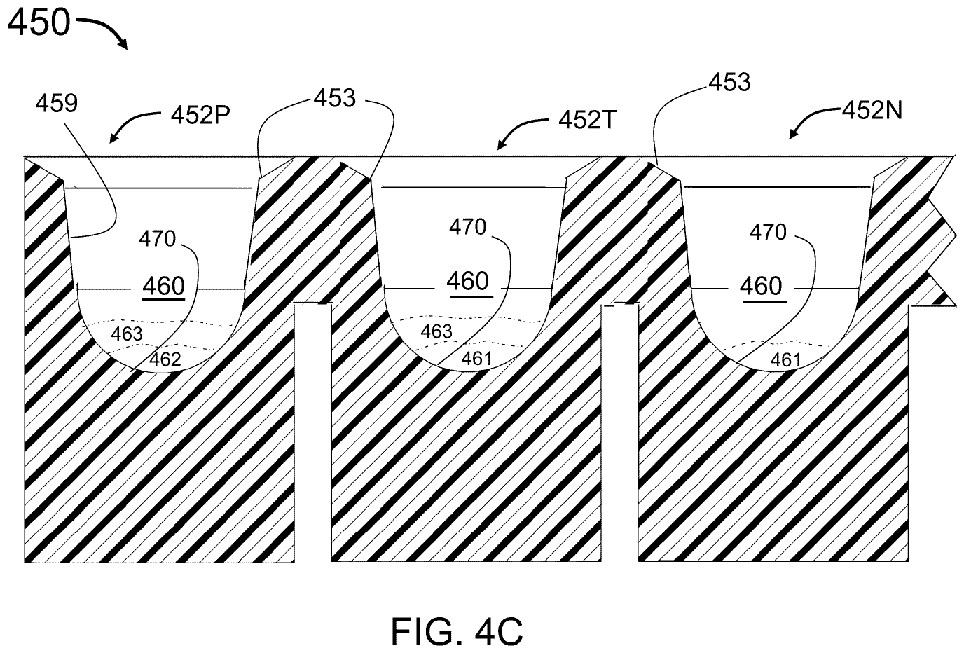

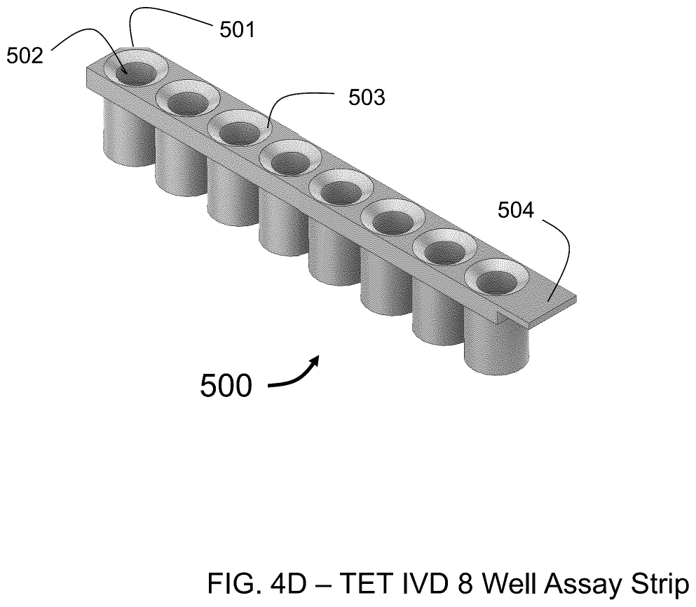

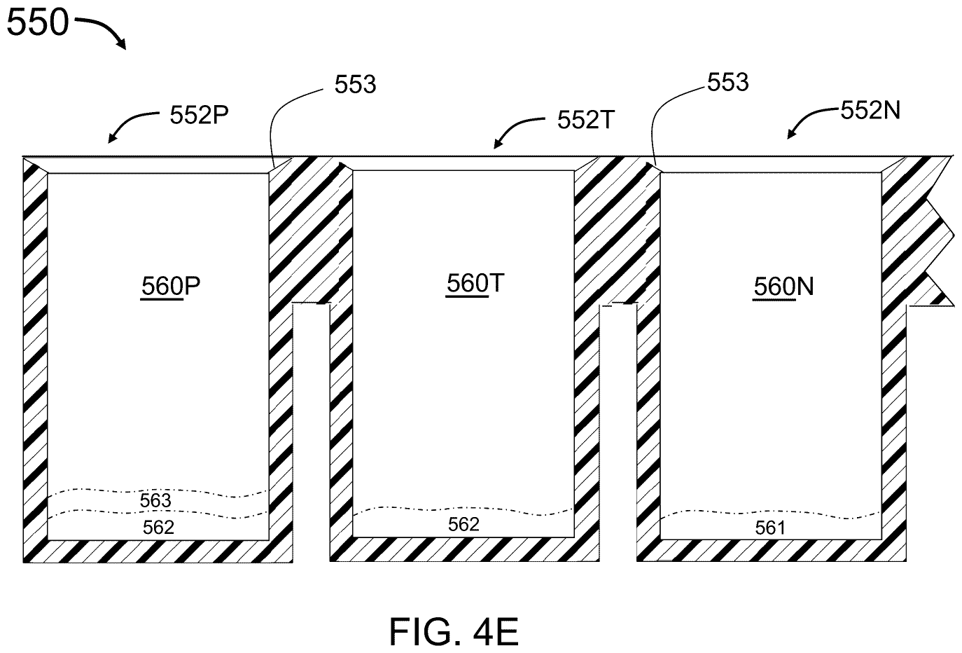

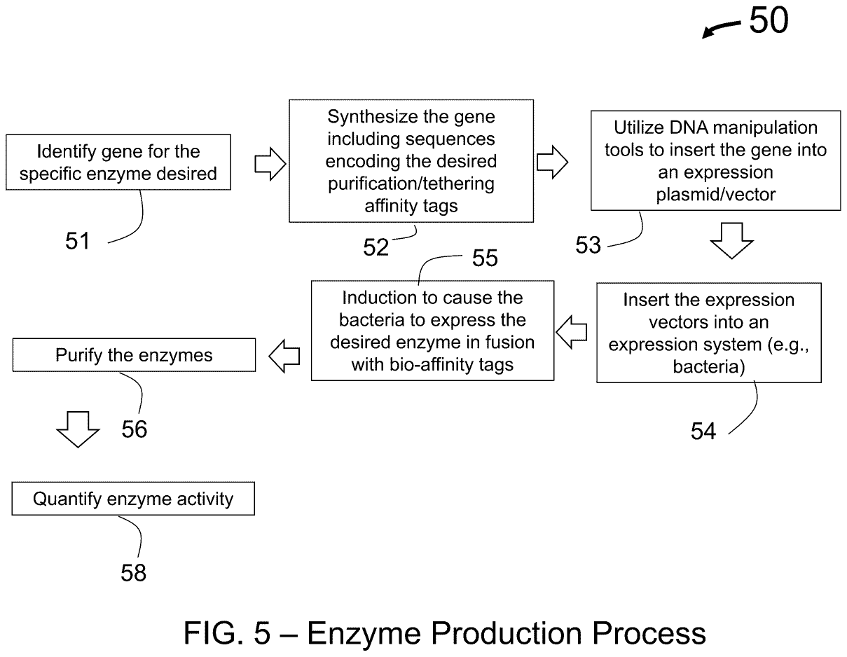

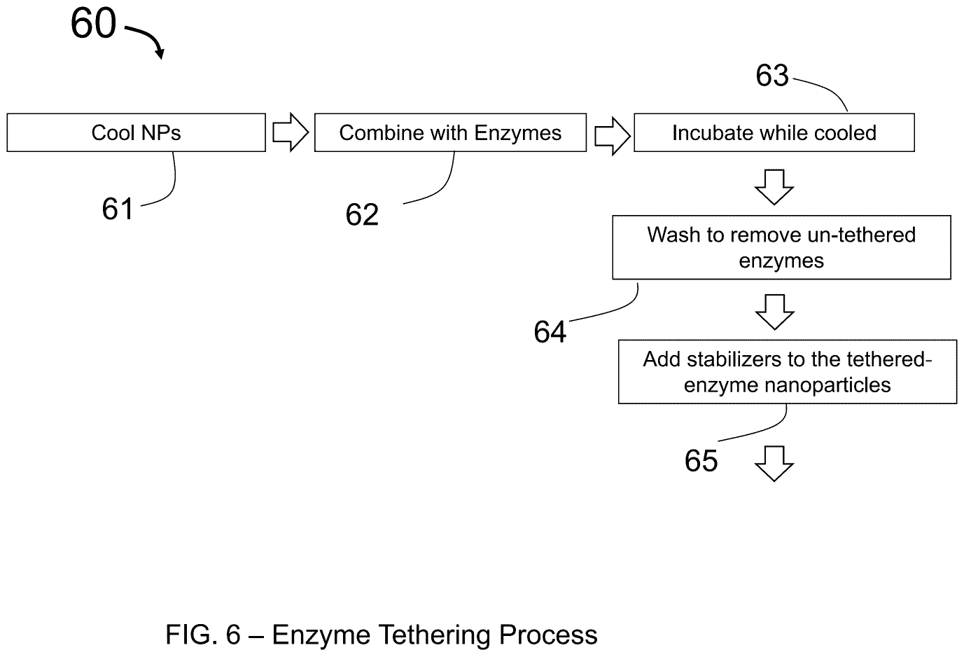

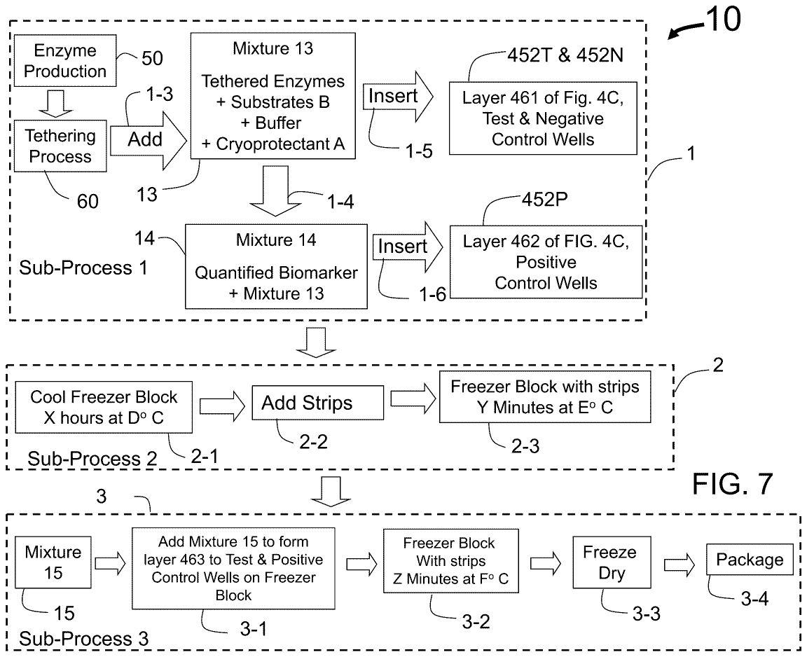

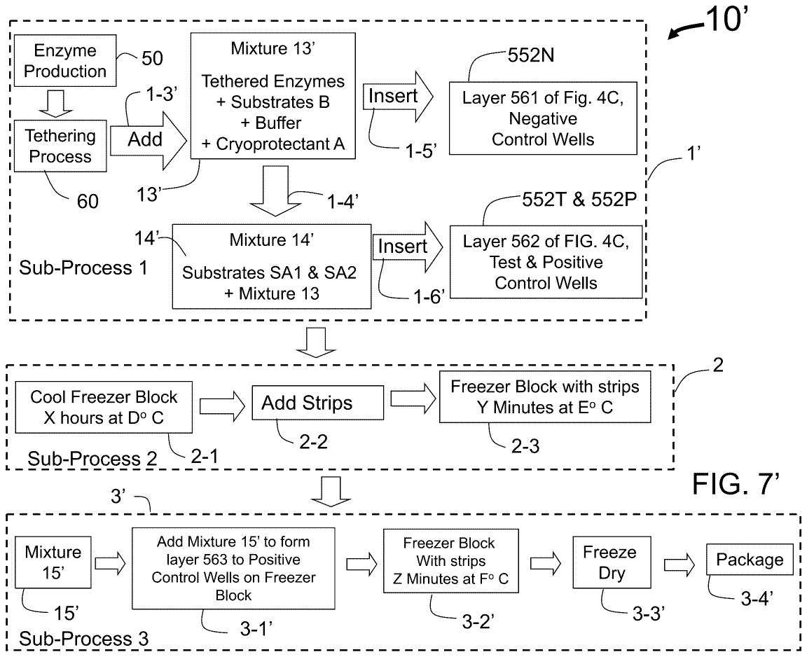

FIG. 4A is a schematic view of an embodiment of the present invention custom assay strip 400 with 12 wells 402, having tapered (chamfered or filleted) entry 403 with an alignment notch 401 and lip 404. The notch 401 and lip 404 provide guides for placing the 12-well strip into a standard plate reader holder so that the left to right orientation of the wells is proper as the contents of the wells may differ. Specifically, if test wells as well as negative and positive control wells are used in a non-symmetric layout, the proper orientation of the strip 400 is critical to interpret results. Note that in preferred embodiments for a luminescence readout, these 12-well strips crafted individually or produced as a portion of a 96-well plate, would be made of a white or reflecting materials so that more photons will leave the well and be captured by a plate reader or photodiode-based detection device. The image in FIG. 4A is shaded to provide easier viewing.

FIG. 4B is a longitudinal cross sectional view of a portion of a three layer component embodiment of the 12-well strip 400 of FIG. 4A showing a test well 402T, a positive control well 402P and a negative control well 402N, each well having a tapered entry 403 and a well volume 420, a curved bottom 410 and underside inset 425 to reduce the amount of plastic needed. The test well 402T has two layers of freeze-dried components 421 and 422 while the positive control well has three layers 421, 422 and 423. The negative control well 402N has only one layer 421. The layer 423 would include a pre-set amount of the biomarker being assayed with examples being an enolase for the NSE-FA assay and ALT or AST for the assays of liver enzymes. The positive control is key to the ability to quantify the enzymatic activity of the biomarker in the patient sample being assayed.

In a preferred embodiment where the biomarker is an enzyme, layer 423 would include the biomarker tethered to silica nanoparticles.

In an embodiment, the 12-well strip 400 would have at least one set of the three wells shown with alternate embodiments of 2 sets, 3 sets or 4 sets. It is also envisioned that other combinations such as one negative control 402N, three positive control wells 402P and three test wells 402T can be used.

In the embodiment where the strip 400 is used for the assay for functionally active NSE-FA shown in FIGS. 3A and 3B, an embodiment is to have the layer 421 include all the components shown in FIG. 2A except the 2-PG substrate 301. The layer 421 is placed in all the wells including test well 402T, negative control well 402N and positive control well 402P. During manufacturing, the layer 421 is placed into the well and then frozen.

For the NSE-FA embodiment of the strip 400 of FIG. 4B, the layer 422 would include the substrate 2-PG 301 of FIG. 3A and would be placed on top of the frozen layer 421 in the test wells 402T and positive control wells 402P but not the negative control wells 402N. Once inserted this layer would also be frozen. The layer 423 for the NSE-FA assay would include a pre-set amount of an active enolase and would be placed only in the positive control wells 402P and quickly frozen to prevent reaction with the frozen 2-PG in layer 422.

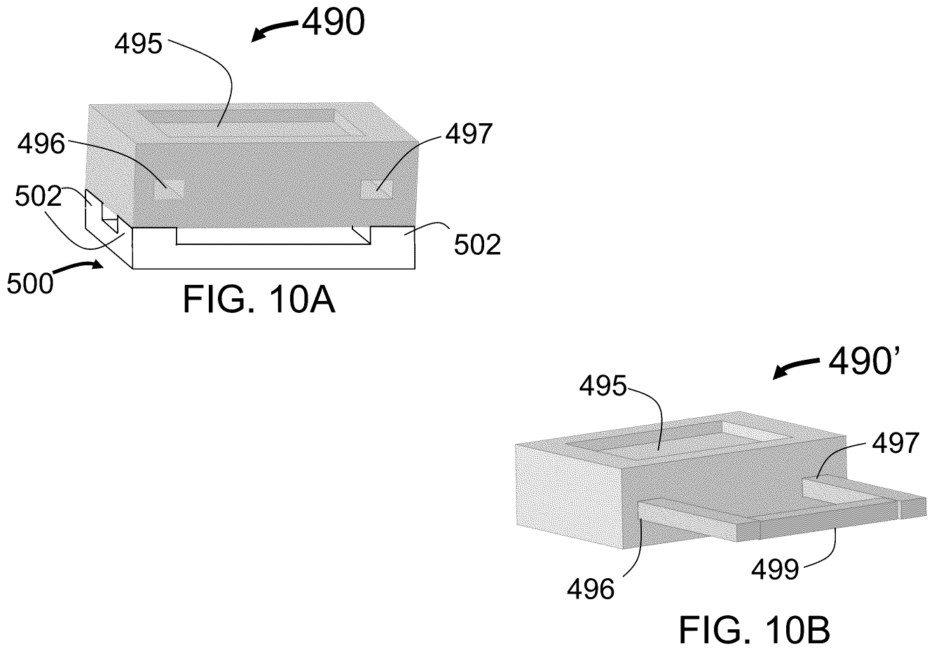

In a preferred embodiment one or more 12-well strips 400 would be placed on a freezer block to maintain the frozen state of the components when layers 422 and 423 are added. In a preferred embodiment, the freezer block such as the freezer block 490 of FIG. 10A would be designed to hold eight strips either individually or in a separate holder.

After all layers are deposited and then frozen, the strip(s) 400 are placed in a lyophilizer to freeze dry the strip(s) 400 that are then sealed in a light blocking and moisture resistant pouch that may optionally include a desiccant as any water that reaches the positive control well 402P could trigger premature reaction.

In an alternate embodiment to the layers 421, 422 and 423 may be placed in the wells in a different order with the biomarker (e.g. enolase) layer 423 placed first in the positive control wells, the primary component group 421 without 2-PG 301 placed next in all wells and the 2-PG 301 layer 422 placed last in the treatment wells 402T and positive control wells 402P. FIG. 7 describes a preferred embodiment of the process for laying down the layers to prevent inadvertent reaction.

It is also envisioned that if a fourth type of well being a second type of negative control well is used, then there would be three sets of four wells. An embodiment of such an added negative control is having a test well that includes a suppressant for NSE-FA but will still react to other forms of enolase from other sources including hemolysis.

FIG. 4C is a cross-sectional view of a two-layer preferred embodiment of the present invention coupled enzyme assay showing the shape of three wells of a 12-well strip 450 with positive control well 452P, test well 452T and negative control well 452N. Each well having a tapered entry 453, a tapered upper well 459, a well volume 460 and a curved well bottom 470. The positive control well 452P has two layers of freeze-dried components 462 and 463. The test well 452T, similar to the test well 402T of FIG. 4B, has two layers of freeze-dried components 461 and 463 while the negative control well 452N, similar to the negative control well 402N of FIG. 4B, has only one layer 461. The advantage of a tapered well top 459 is to better allow for mold release without use of chemicals.

In the embodiment where the strip 450 is used for the assay for functionally active NSE-FA described in FIGS. 3A and 3B, a preferred embodiment is to have the layer 461 include all the components shown in FIG. 2A except the 2-PG substrate 301. Layer 461 is placed in test wells 452T and negative control wells 452N.

In the embodiment where the strip 450 is used for the assay for ALT or AST described in FIG. 3C, a preferred embodiment is to have the layer 461 include the components Luminol and L-Alanine for ALT and Luminal and L-Aspartate for AST. Layer 461 is placed in test wells 452T and negative control wells 452N.

The layer 462 in the positive control wells would include the components in layer 461 plus a preset amount of the biomarker desired (e.g. an Enolase for the NSE-FA Assay, ALT or AST for liver enzyme assays). Layer 462 would be placed as a first layer of each positive control wells 452P. In a preferred embodiment where the biomarker is an enzyme, layer 462 would include the biomarker tethered to silica nanoparticles. For the NSE-FA embodiment the layer 462 would include an enolase tethered to silica nanoparticles. An alternate embodiment would use un-tethered freeze-dried enolase in layer 462.

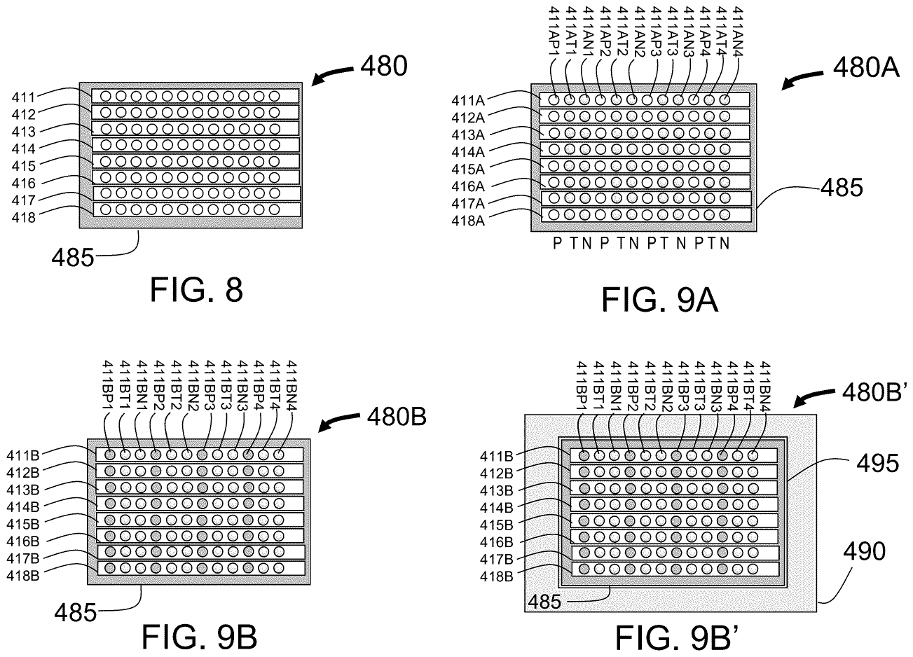

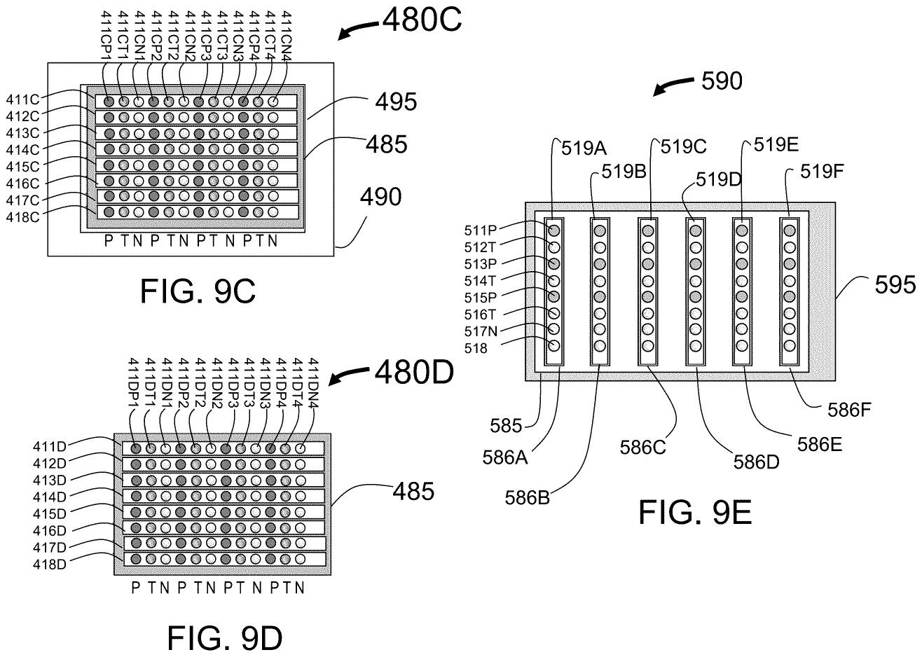

In a preferred embodiment, eight strips would be placed into a multi-strip holder, layers 461 and 462 would be placed into the wells at temperatures between 4° C. and 25° C. then a freezer block described (e.g., 490 of FIG. 9B′, 9C, 10A and 10B) would be removed from a freezer and the multi-strip holder (e.g., 485 of FIG. 8) with the strips 450 would be placed onto the freezer block 490 and frozen at −15° to −100° C. for 5 to 30 minutes with a preferred embodiment being for at least 15 minutes in a less than −70° C. (e.g. a −80° C.) freezer.

After the first layer (461 or 462) is deposited, the entire strip 450 is placed in a freezer for a specified time between 1 and 30 minutes in a −15° to −100° C. freezer, with a preferred embodiment being for at least 15 minutes in a less than −70° C. (e.g. a −80° C.) freezer.

After removing the frozen strip(s) 450 from the freezer, the second layer 463 is placed on top of the frozen first layer 461 in the test wells 452T and on top of the frozen first layer 462 of the positive control well(s) 452P but not the layer 461 of the negative control well(s) 452N. Once deposited, layer 463 would then be frozen for a specified time between 1 and 30 minutes in a −15° to −100° C. freezer, with a embodiment being for at least 15 minutes in a less than −70° C. (e.g. a −80° C.) freezer.

For the embodiment for NSE-FA, layer 463 would include the substrate 2-PG of FIGS. 3A and 3B as well as buffers and cryoprotectants. For the embodiment for ALT and AST the layer 463 would include the substrate a-ketoglutarate as well as buffers and cryoprotectants.

After all layers are deposited and then frozen, the strip(s) 450 are placed in a lyophilizer to freeze dry the strip(s) 450 that are then sealed in a light blocking and moisture resistant pouch that may optionally include a desiccant as any water that reaches the positive control well 452P could trigger premature reaction.

In a preferred embodiment where the biomarker is an enzyme, layer 462 would include the biomarker tethered to silica nanoparticles. For the NSE-FA embodiment the layer 462 would include an enolase tethered to silica nanoparticles.

In an alternate embodiment the layers may be placed in the wells in a different order with the layer 463 placed first in the positive control well(s) 452P and test well(s) 452T and the strip frozen. Then the layer 462 of the positive control well(s) 452P and the layer 461 of the test and negative control wells 452T and 452N would be added.

While the layers can be reversed, a preferred embodiment of the present invention has layer 463 that includes the substrates with which the enzyme biomarker reacts on top of the base layers 462 and 461 so layer 463 is that the first layer contacted when the sample is placed in the well. This facilitates a faster reaction start up allowing for better and quicker separation of the luminescence curves the test and positive control reactions.

FIG. 4D is a schematic view of an embodiment of the present invention custom assay strip 500 with 8 wells 502, having tapered (chamfered or filleted) entry 503 with an alignment notch 501 and lip 504. The notch 501 and lip 504 provide guides for placing the 12-well strip into a standard plate reader holder so that the left to right orientation of the wells is proper as the contents of the wells may differ. Note that in preferred embodiments for a luminescence readout, these 8-well strips crafted individually or produced as a portion of a 96-well plate, would be made of a white, light colored or reflecting materials so that more photons will leave the well and be captured by a plate reader or photodetector-based point-of-care reader. The image in FIG. 4D is shaded to provide easier viewing.

In a preferred embodiment of the present invention 8-well strip 500, the 8 wells would have three positive control/reference wells, three test wells and two negative control/reference wells. This will allow one to place 12 strips instead of 8 into a 96 well holder reducing the cost of production. There is little real advantage in 4 wells over three as with three one can average them together if they are close or eliminate one of the three if it appears to be an outlier and average the other two, if for example, the pipetting of fluid accidentally introduced too much or too little of the test sample into one well.

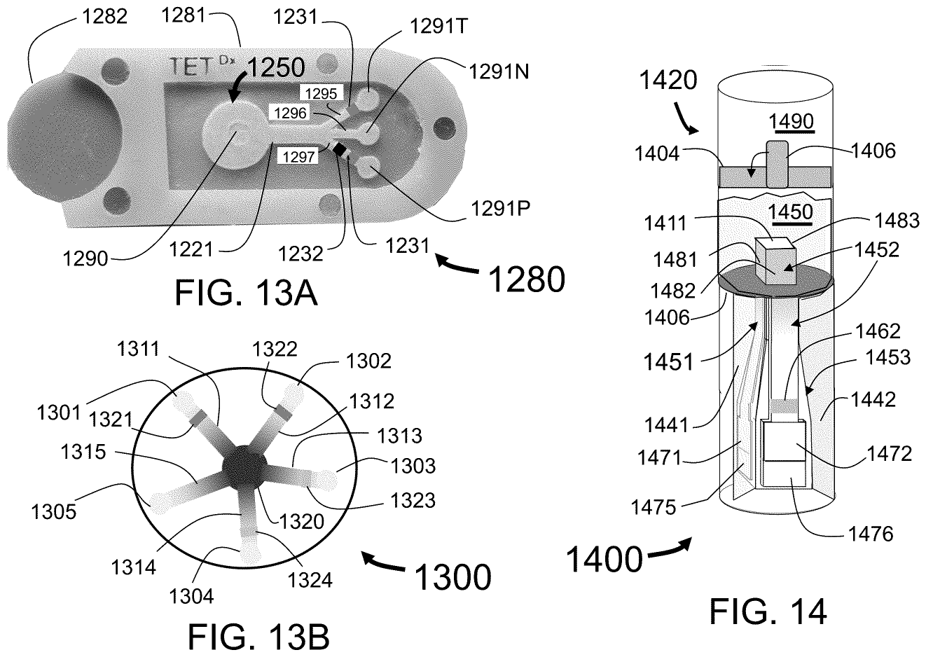

In another preferred embodiment, the inside surface of the wells 402 of FIGS. 4A and 502 of FIG. 4D have a coating (for example poly-ethylene-glycol (PEG), poly-ethylene-oxide (PEO), or tween) to prevent inadvertent attachment of proteins to the surface of the wells 402 and 502. Similar coatings can be helpful in PoC embodiments of test strips or cards such as shown in FIGS. 13A, 13B and 22.

Use of freeze drying in the preparation of Coupled Tethered Enzyme Luminescence Assays (CTELA) having positive controls or high/low references requires packaging in a low humidity environment to prevent premature reactions.

FIG. 4E is a cross-sectional view of a two-layer preferred embodiment of the present invention coupled enzyme assay having only two layers in the positive control wells with the second layer being enolase instead of 2-PG as in FIG. 4C. FIG. 4E shows the cylindrical shape 559 of three wells of an 8-or 12-well strip 550 with positive control well 552P, test well 552T and negative control well 552N. Each well having an optional tapered entry 553 and well volumes 560P, 560T and 560N. The positive control well 552P has two layers of freeze-dried components 562 and 563. The test well 552T has one layer of freeze-dried components 562 and the negative control well 552N, similarly has only one layer 561. A tapered well entry 553 may provide for better mold release without use of volatile or other chemical mold-release agents. While the positive control well 552P is shown with lower or first layer 562 that is the same as the only layer 562 in the test well 552T, it is envisioned that the second layer 563 that includes a pre-set amount of biomarker may be placed as the first or lower layer.

In a preferred embodiment an ATK inhibitor is included in layers 561 of the negative control well 552N and 562 of the test and positive control wells 552T and 552P.

In the embodiment where the strip 550 is used for the assay for functionally active NSE-FA described in FIGS. 3A and 3B,
A preferred embodiment is to have the layer 561 in the Negative Control well 552N include all the components shown in FIG. 2A except the 2-PG substrate 301.
Layer 562 in the test well 552T and positive control well 552P has all the components of the layer 561 of the negative control well 552N plus the addition of 2-PG;
The layer 563 of the positive control well 552P includes a pre-set amount of an enolase enzyme such as NSE, NNE or another form of enolase. The enzymes may be freeze dried directly or freeze dried after tethering to a surface such as a silica nanoparticle.

In a preferred embodiment where the strip 550 is used for the assay for ALT or AST described in FIG. 3C,
a preferred embodiment is to have the layer 561 include the components Luminol and L-Alanine for ALT and Luminol and L-Aspartate for AST. Layer 561 is placed in the negative control wells 552N.
The layer 562 in the positive control wells 552P and test wells 552T would include the components in layer 561 plus a-ketoglutarate for both ALT and AST.
The layer 563 of the positive control well 552P includes a pre-set amount of an ALT or AST. The enzymes ALT or AST may be freeze dried directly or freeze dried after tethering to a surface such as a silica nanoparticle.

After the first layer (561 or 562) is deposited, the entire strip 550 is placed for a specified time between 1 and 30 minutes in a −15° to −100° C. freezer, with a preferred embodiment being for at least 15 minutes in a less than −70° C. (e.g. a −80° C.) freezer.

In an embodiment, up to twelve with a preferred embodiment of six strips would be placed into a multi-strip holder, layers 561 and 562 would be placed into the wells at temperatures between 4° C. and 25° C. then a freezer block like that described (e.g., 490 of FIG. 9B', 9C, 10A and 10B) would be removed from a freezer and the multi-strip holder (e.g., 485 of FIG. 8) with the strips 550 would be placed onto the freezer block 490 and frozen at −15° to −100° C. for 5 to 30 minutes with a preferred embodiment being for at least 15 minutes in a less than −70° C. (e.g. a −80° C.) freezer.

After removing the frozen strip(s) 550 on the freezer blocks from the freezer, the second layer 563 is placed onto or adjacent to of the frozen first layer 562 of only the positive control well(s) 552P. Once deposited, layer 563 would then be frozen for a specified time between 1 and 30 minutes in a −15° to −100° C. freezer, with a preferred embodiment being for at least 15 minutes in a less than −70° C. (e.g. a −80° C.) freezer.

For the embodiment for NSE-FA, layer 563 would include a preset amount of the biomarker or a similar item (e.g. enolase for NSE) as well as buffers and cryoprotectants.

Although FIGS. 4B, 4C and 4E show layers in the wells as on top of each other, when the second or third layers are deposited into the well after the prior layer(s), in reality, the layers may end up with one or more portions adjacent to or on the side of a prior layer. For the purposes of the embodiments of the present invention, the term onto or on top of will include such adjacent configurations.

After all layers are deposited and then frozen, the strip(s) 550 are placed in a lyophilizer to freeze dry the strip(s) 550 that are then sealed while exposed to an inert, dry gas (e.g. nitrogen or argon) at a pressure below 1 atm so as to cause the package seal to flex inward when the sealed package is back at ambient 1 atm pressure. This concave shape of the seal over each well will provide an easy warning that the seal is damaged if the inwardly-flexed shape is not visible.

In a preferred embodiment, the inside surface of the wells 402 of FIGS. 4A, 402P, 402T and 402N of FIGS. 4B, 502 of FIGS. 4D and 552P, 552T and 552N of FIG. 4E have a coating; for example poly-ethylene-glycol (PEG), poly-ethylene-oxide (PEO), tween or other coatings to prevent inadvertent attachment of proteins to the surface of the wells 402 and 502

FIG. 5 is a block diagram of a preferred embodiment of the enzyme production process 50 using an expression system (e.g., bacteria) to produce enzymes designed for tethering.
The process begins by identification in step 51 of the specific gene that will encode expression by bacteria of the desired enzyme with affinity tags for tethering. Production of the enzymes to be tethered by introduction of coding genetic material (e.g., DNA or RNA sequences) into a biological entity or expression system that will then produce the needed enzymes with two different types of affinity tags with one type designed to facilitate extraction from the expression system and the other suitable for tethering. The biological entity may be mammalian cells, insect cells, yeast or bacteria. The system may also be a “cell-free expression system” containing elements typically found in one or more of the above entities or completely artificial (e.g., protein printing). One or more follow-on purification steps may be included. Using two different affinity tags allows for higher purity of the enzymes tethered to the nanoparticles. One preferred embodiment of this method of production is having both a 6-histidine tag (6xHis) for extraction/purification and a silicon dioxide (SiO₂) tag for tethering to a silica nanoparticle as the two affinity tags.
The gene in fusion with one or more affinity tags to allow purification and tethering as shown in FIG. 6 and purification in step 56 is synthesized in step 52, for example, using a DNA synthesizer.
Next, one inserts in step 53 the gene encoding one or more affinity tags into an expression plasmid/vector.
Next, one inserts the expression plasmid in step 54 into a protein expression system. Examples of expression systems include bacteria, insect cells, mammalian cells, yeast or other known expression systems. Bacteria are used in the preferred embodiment of the process 50.
Next in step 55 the bacteria are induced to produce/express the desired enzyme including the desired affinity fusion tags.
The final step is the purification process 56 that is used to separate other materials from the desired enzymes.

FIG. 6 is a block diagram of an embodiment of the enzyme tethering process 60 where active enzymes are tethered to nanoparticles (e.g., silica (SiO₂) nanoparticles) using oriented immobilization that increases the stability and activity of the enzymes to facilitate improved shelf life and faster reaction times than can be achieved with non-oriented enzymes. This novel process produces an oriented immobilization that improves substrate access to the substrate-binding site/active site of each enzyme molecule and/or enables improved conformational changes or movements, and/or improves substrate channeling to a subsequent reaction step. The tethering process 60 follows enzyme purification step 56 of the Enzyme Production Process 50 of FIG. 5 as follows:
The process begins in step 61 by cooling the nanoparticles in a vessel to a temperature between 1 and 10 degrees Celsius.
Next in step 62, the purified enzymes with the affinity tags are added to the vessel.
Next in step 63 the cooled vessel is allowed to incubate for a pre-set period of time. For example a period of 15 to 60 minutes may be used.
Next in step 64 while still cooled, the incubated mixture is washed to remove un-tethered enzymes. For example. the nanoparticles are spun down using a centrifuge to the bottom of the vessel and the remaining liquid is replaced with a buffer such as phosphate buffer. This is repeated 1 to 5 times. In a preferred embodiment, the spin-down speed should for example be between 300 g to 1000 g. In some cases, 2 or more different enzymes may be combined in step 62 where it is desired that more than one type of enzyme is tethered to each nanoparticle. It is also envisioned that one can control the number of enzymes per nanoparticle by controlling the amount of enzymes added in step 62 relative to the size, number and concentration of nanoparticles.
In the final step 65, stabilizers are added to the tethered nanoparticles. For example, suitable stabilizers include sorbitol, dextran, polyethelyne glycol or trehalose, among others.

It is envisioned that nanoparticles with other composition (other than silica) or other structures may also be used with the tethering process 60 to provide a surface for tethering enzymes.

While the prior art Travis embodiments describe use of two type of affinity tags to allow tethering to two types of surfaces, the present invention envisions a preferred embodiment with one type of affinity tag for tethering with oriented immobilization to a surface (e.g., a silica nanoparticle) and use of a second type of affinity tag to facilitate extraction of the enzymes from the expression system. Using two different affinity tags allows for higher purity of the enzymes tethered to the nanoparticles. An example of such a preferred embodiment as the affinity tags being a 6xHis tag and a SiO₂ tag.

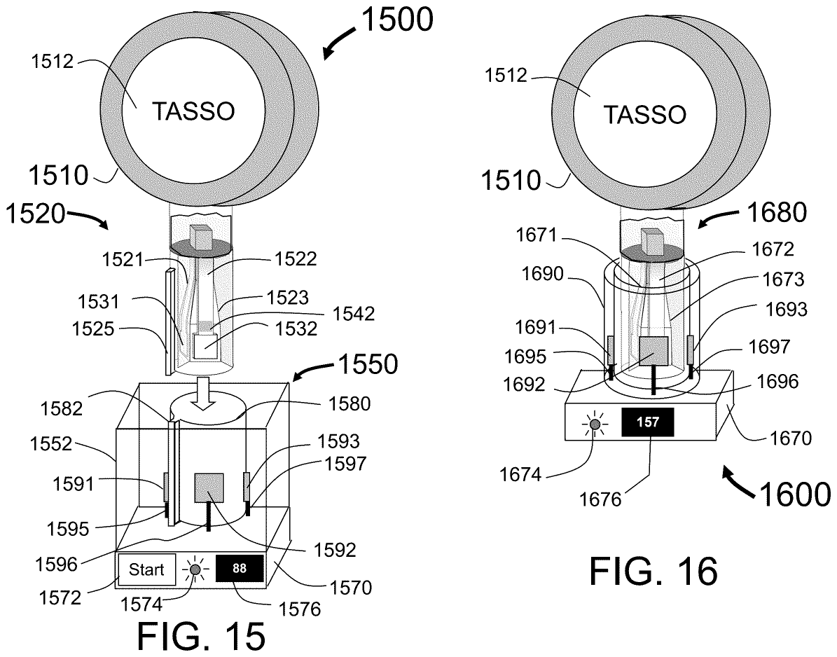

FIG. 7 is a block diagram of an example of a preferred embodiment of the production process 10 for the assay strips 400 of FIG. 4A, including the steps for inserting the materials that are placed into the wells/zones for the present invention TET coupled enzyme assay 100 of FIG. 1. Such an assay is designed to receive a sample that may include an enzyme biomarker being assayed and can be read using standard lab plate readers (e.g., the TECAN Infinite 200 PRO), photodiode or silicon photomultiplier-based readers as shown in FIGS. 15,16, and 23 or a reader being integrated with a TET-PoC point of care assay device designed to accept vials of a fluid such as shown in FIG. 19. A preferred embodiment of the mixture 13, mixture 14 and mixture 15 of FIG. 7 form respectively the layers 461, 462 and 463 of FIG. 4C.

A preferred embodiment of the present invention TET coupled enzyme assay would include at least one test well or zone, at least one positive control well or zone and at least one negative control well or zone.

A preferred embodiment of the present invention production process 10 to produce the strip 450 of FIG. 4C begins with Sub-Process 1 where a preferred embodiment process is performed at 4 degrees Centigrade comprising the following steps
1. The first step is the enzyme production process 50 of FIG. 5;
2. Next is the enzyme tethering process 60 of FIG. 6;
3. The tethered enzymes are then added in step 1-3 to the other materials including buffers, cryoprotectants and the substrates SB3 123, and SC3 133 shown in FIG. 1. These together form mixture 13 being the mixture used as the only layer for the negative control well(s) and the base layer for the test well(s) (e.g. layer 421 of FIG. 4B or layer 461 of FIG. 4C). Examples of the buffer components and cryoprotectants include sorbitol, dextran, trehalose, glycerol; potassium ions, magnesium ions, phosphate ions, sodium ions and/or polyethylene glycol (PEG). For an embodiment of the NSE-FA assay of FIG. 3A, mixture 13 includes ADP (SB3 123) and luciferin (SC3 133) but does not include SA1 111 the primary initial substrate 2-PG needed to start the coupled enzyme reaction once in contact with a patient sample having NSE-FA. It is also envisioned that while SA1 111 of FIG. 1
4. Next in step 1-4 a portion of mixture 13 is added to a quantified amount of the biomarker being assayed to form Mixture 14. This is the base layer 462 of FIG. 4C for the positive control well(s). For the NSE-FA assay, the quantified biomarker added is an enolase that in a preferred embodiment may be tethered.
5. At this point, once mixtures 13 and 14 are ready, in step 1-5 a pre-set quantity of the negative control mixture 13 is inserted as layer 461 into the test well(s) 452T and negative control well(s) 452N of FIG. 4C.
6. This is followed in step 1-6 where the mixture 14 is inserted as Layer 462 into the positive control well(s) 452P of FIG. 4C or zone(s), completing sub-process 1.

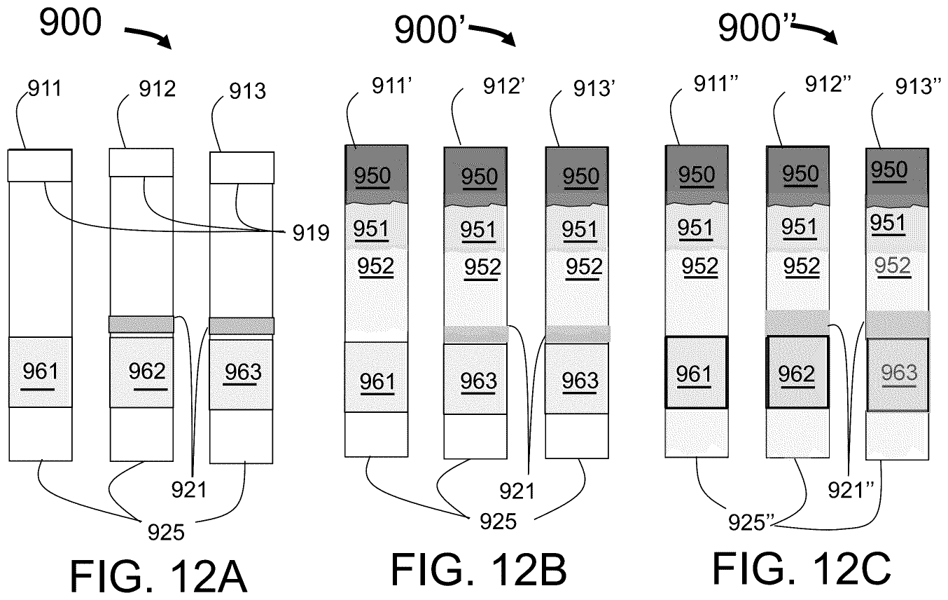

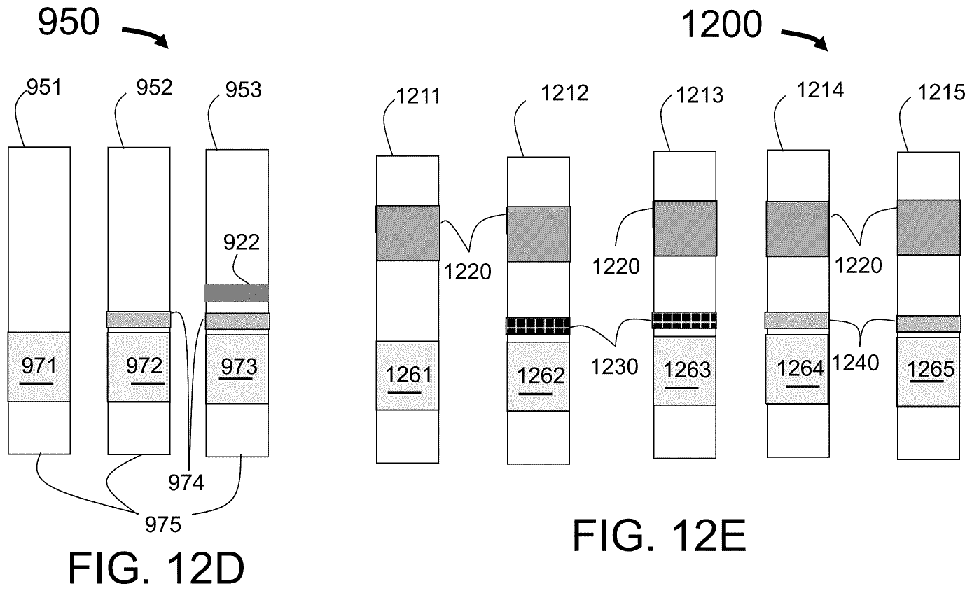

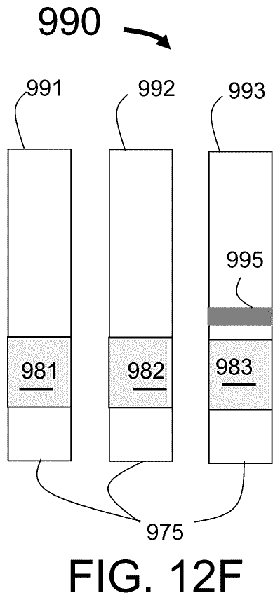

Sub-Process 1 is followed by Sub-Process 2 with steps as follows:
1. To begin Sub-Process 2, in step 2-1, a freezer block (e.g., the freezer block 490 shown in FIGS. 10A and 10B), is placed in a freezer for at least X hours at D degrees centigrade. The freezer block 490—for example might be a piece of aluminum adapted to hold one or more strips 400 of FIGS. 4A-4C or blood separation chromatography strips (e.g., the strips 900 of FIG. 12A). In a preferred embodiment, X is typically more than 6 hours and D is less than −15 degrees centigrade with a preferred embodiment being 24 hours and less than −70 degrees centigrade (e.g., a −80° C. freezer).
2. Next in step 2-2 the freezer block is removed from the freezer and the strips prepared with negative control, test and positive control first layers (e.g., layers 461 and 462 of FIG. 4C) are placed in contact with the freezer block. In a preferred embodiment up to eight 12-well strips are in a holder (e.g., the holder 485 in FIG. 8 for the 8 IVD strip 400 of FIG. 4A).
3. Next in step 2-3, the freezer block with strips is placed back in a freezer for Y minutes below E degrees centigrade. In one embodiment Y is more than 10 minutes and E is −15 degrees centigrade. In a preferred embodiment, Y is 20 minutes and E is −70 degrees centigrade (e.g. a −80° C. freezer). This completes Sub-Process 2.

The final Sub-Process 3 in the preferred embodiment for production of the present invention coupled enzyme assay comprises the steps as follows:
1. Once the first layers (e.g., 461 or 462 of FIG. 4C) are fully frozen and the strip(s) are still in contact with the freezer block, in step 3-1 of Sub-Process 3, Mixture 15 is inserted to form layer 463 of FIG. 4C for the positive control well(s) 452P and the test well(s) 452T of FIG. 4C. Mixture 15 includes the substrate(s) that will be acted upon by the enzyme biomarker being assayed to begin the reaction ending in a signal output (e.g., the production of luminescence). For the NSE-FA assay, Mixture 15 would include 2-PG. For liver enzymes ALT and AST, Mixture 15 would include a-ketoglutarate. Mixture 15 may also include buffers and cryoprotectants (e.g., sorbitol, dextran, trehalose, glycerol and PEG).
2. Next in step 3-2, the freezer block with the complete formulation for the assay wells or zones, is placed back in the freezer for Z minutes at below F degrees centigrade. In an embodiment Z may more than 10 minutes and F is −15 degrees Centigrade. In a preferred embodiment, Z is 20 minutes and F is −70 degrees centigrade (e.g., a −80° C. freezer).
3. Next, in step 3-3, the strip(s) 450 of FIG. 4C with the fully frozen complete formulation of test well(s) 452T, negative control well(s) 452N and positive control well(s) 452P, is placed in a freeze dryer to remove all water or other liquid in the well(s) that could allow the positive control well(s) to begin reacting without the addition of the patient liquid sample(s) to be tested.
4. The final step 3-4 is to remove the strips from the freeze dryer in a low humidity environment and package them in a sealed pouch. In a preferred embodiment a desiccant is placed in the pouch to ensure that no moisture reaches the well(s).

It is also envisioned that the positive control well(s) could be prepared with only the end stage substrates SC3 133 and SC1 131 needed for activation of the signaling tethered enzyme TE2 130 of FIG. 1. For the NSE-FA coupled enzyme assay, this would have the positive control with a first layer including tethered luciferase and luciferin and a second layer with a pre-set quantity of Adenosine Tri-Phosphate (ATP). Similarly, a positive control with the first layer being the composition of the negative control layer 461 of FIG. 4C with a second layer including a pre-set amount of the substrate SB1 121 of FIG. 1 that would be Phosphoenolpyruvate (PEP) for the NSE-FA assay.

It is also envisioned that there could be two or more positive control types with one having the amount of the biomarker (e.g., enolase activity) at the threshold for brain injury and one having a much higher amount (e.g., ten times), to provide calibration and enhance quantification between the two levels.

In another embodiment, the positive control well(s) can be replaced by previously-obtained, temperature-dependent luminescence data recorded from positive controls, or the TET-IVD strip could include an LED that provides a light output over time that may be temperature adjusted to emulate the result that is seen by recorded positive control wells when activated. It is also envisioned that the recorded signal from a positive well could be stored in the TET-PoC memory and no positive well(s) would be needed.

In another embodiment for the point of care TET-PoC NSE-FA assay with electrical voltage signal output, the tethered luciferase (LUC-NP) is replaced by the enzyme pyruvate oxidase tethered to silica nanoparticles (PYROX-NP), for preparation of the test wells, and positive control and negative control wells; otherwise, the multi-step process is highly similar.

In another embodiment for electrical signal output pyruvate oxidase would be tethered directly onto an electrode (preferably silver or gold). In this embodiment, the Si-tag on the pyruvate oxidase would be replaced with either an Ag-tag or Au-tag.

While the present invention embodiments include the use of PK enzymes and ADP as a substrate to produce ATP as the intermediate components in the assay, other embodiments can instead use
Pyruvate Phosphate Di-Kinase (PPDK) converts Adenosine Monophosphate (AMP) and Phosphate into ATP
Phosphoenolpyruvate carboxykinase (PEPCK) converts PEP and ADP into ATP.

In another embodiment, the pyruvate oxidase will be tethered to SiO₂ nanoparticles or to an electrode, and HRP will be immobilized onto an electrode (using Au-or Ag-tags, for example).

It is also envisioned that cofactors FAD and TDP could be added (but not required). Byproducts of hemolysis can adversely affect the measurements of a biomarker in a patient sample. There are two primary causes, one is the change in color to a pink or red for higher levels of hemolysis, that can affect transmission/absorbance of light. The other is the biological activity of components coming out of damaged blood cells. For the NSE-FA assay, the main interfering component coming from hemolyzed red blood cells (RBCs) is the enzyme Adenylate Kinase (ADK), which in the forward reaction acts on molecules of ADP to produce ATP. To reduce ADK interference effects coming from hemolysis, a preferred embodiment of the Mixture 13 would include the addition of an ADK inhibitor. This will then also have the ADK inhibitor be included in the subsequent mixtures 14 and 15.

These changes to the mixture 13 composition will allow for meaningful results from blood samples with hemolysis levels up to 3 on the CDC Hemolysis Reference Palette (within the range of 20 to 50mg/dL hemoglobin).

While it is possible to produce 1 strip at a time, this is not practical for the size and scope of the unmet need for the advanced assays possible with the present invention. The next figures and description will describe a production method to produce multiple strips efficiently for commercial or clinical applications. This technique is applicable for production with various automated or semi-automated injection systems such as (but not limited to) the Hamilton Microlab NIMBUS or STAR, or the Opentrons OT-2 or other Workstations.

FIG. 7′ is a block diagram of an example of a preferred embodiment of the production process 10′ for the assay strips 500 of FIG. 4D, including the steps for inserting the materials that are placed into the wells/zones for the present invention TET coupled enzyme assay 100 of FIG. 1. Such an assay is designed to receive a sample that may include the enzyme biomarker being assayed and can be read using standard lab plate readers (e.g. the TECAN Infinite 200 PRO), a photodiode or SIPM based plate reader as shown in FIGS. 15, 16, and 23 or by a reader (FIG. 19) integrated into a point of care assay device. A preferred embodiment of the mixture 13, mixture 14′ and mixture 15′ of FIG. 7′ form respectively the layers 561, 562 and 563 of FIG. 4E.

A preferred embodiment of the present invention TET coupled enzyme assay would include at least one test well or zone, at least one positive control well or zone and at least one negative control well or zone.

A preferred embodiment of the present invention production process 10′ to produce the strip 550 with test, positive and negative control wells shown in FIG. 4E begins with sub-process 1′ where a preferred embodiment process is performed at 4 degrees Centigrade comprising the following steps
1. The first step is the enzyme production process 50 of FIG. 5;
2. Next is the enzyme tethering process 60 of FIG. 6;
3. The tethered enzymes are then added in step 1-3′ to the other materials including buffers, cryoprotectants and the substrates SB3 and SC3 shown in FIG. 1. These together form mixture 13′ being the mixture used as the only layer for the negative control well(s). Examples of the buffers and cryoprotectants include sorbitol, dextran, trehalose, glycerol; potassium ions, magnesium ions, phosphate ions, sodium Ions and/or PEG. For an embodiment of the NSE-FA assay of FIG. 3A, mixture 13′ includes ADP (SB3 123) and luciferin (SC3 133) but does not include 2-PG (SA1 111) and for ALT/AST SA2 112 as the primary initial substrate needed to start the coupled enzyme reaction once in contact with a patient sample.
4. Next in step 1-4′ a portion of mixture 13′ is added to a quantified amount of the substrates SA1 111 and SA2 112 of FIGS. 1 and 2 as the primary initial substrate needed to start the coupled enzyme reaction once in contact with a patient sample to form Mixture 14′. This is the base layer 562 of FIG. 4E for the test well(S) 552T and positive control well(s) 552P of FIG. 4E. For the NSE-FA assay, the substrate added is 2-PG, For the ALT assay, the substrates are α-ketoglutarate and L-Alanine and for AST α-ketoglutarate and L-Aspartate. It is also envisioned that for the ALT and AST assays, SA2 112 may be included in Mixture 13′ and SA1 111 in Mixture 14′ or vice versa so long as Mixture 13′ does not have both it will not react with ALT or AST in the negative control wells.
5. At this point, once mixtures 13′ and 14′ are ready, in step 1-5′ a pre-set quantity of the negative control mixture 13′ is inserted as layer 561 into the negative control well(s) 552N of FIG. 4E.
6. This is followed in step 1-6′ where the mixture 14′ is inserted as Layer 562 into the positive control well(s) 552P and test well(s) of FIG. 4E or zone(s) completing sub-process 1′.

Sub-process 1′ is followed by sub-process 2 (identical to sub-process 2 of FIG. 7) with steps as follows:
1. First, in step 2-1 a freezer block (e.g., the freezer block 490 shown in FIGS. 10A and 10B), is placed in a freezer for at least X hours at D degrees centigrade. The freezer block 490—for example might be a piece of aluminum adapted to hold one or more strips 400 of FIGS. 4A-4C or blood separation chromatography strips (e.g., the strips 900 of FIG. 12A. In a preferred embodiment, X is typically more than 6 hours and D is less than −15 degrees centigrade with a preferred embodiment being 24 hours and less than −70 degrees centigrade (e.g. a −80° C. freezer).
2. Next in step 2-2 the freezer block is removed from the freezer and the strips prepared with negative control, test and positive control first layers (e.g., layer 561 and 562 of FIG. 4E) are placed in contact with the freezer block. In an embodiment up to twelve 8-well strips are placed in a 96 well plate shaped holder. In a preferred embodiment 6 8-well strips 500 of FIG. 4D are placed in a strip package (e.g., the package 585 of FIG. 9E) and the package is placed in a holder (e.g. the holder 595 of FIG. 9E.
3. Next in step 2-3, the freezer block with strips is placed back in a freezer for Y minutes below E degrees centigrade. In one embodiment Y is more than 10 minutes and E is −15 degrees centigrade. In a preferred embodiment, Y is 20 minutes and E is −70 degrees centigrade (e.g. a −80° C. freezer). This completes sub-process 2.

The final sub-process 3′ in the preferred embodiment for in the production of the present invention coupled enzyme assay comprises the steps as follows:
1. Once the first layers (e.g. 561 or 562 of FIG. 4E are fully frozen and the strip(s) are still in contact with the freezer block, in step 3-1′ of sub-process 3′, Mixture 15′ is inserted to form layer 563 of FIG. 4E for the positive control well(s) 552P of FIG. 4E. Mixture 15′ includes a preset amount of the biomarker being assayed. For example, for the NSE assay, an enolase enzyme (e.g. NSE or NNE, enolase alpha, gamma etc.) would be used. For ALT and AST, a preset amount of ALT or AST respectively would be used. Mixture 15′ may also include buffers and cryoprotectants (e.g. sorbitol, dextran, trehalose, glycerol and PEG).
2. Next as in step 3-2 of FIG. 7, in step 3-2', the freezer block with the complete formulation for the assay wells or zones, is placed back in the freezer for Z minutes at below F degrees centigrade. In an embodiment Z may be more than 10 minutes and F is less than −15 degrees Centigrade. In a preferred embodiment, Z is 20 minutes and F is less than −70 degrees centigrade (e.g. a −80° C. freezer).
3. Next, in step 3-3′, the strip(s) 550 of FIG. 4E with the fully frozen complete formulation of test well(s) 552T, negative control well(s) 5552N and positive control well(s) 552P, is placed in a freeze dryer to remove all water or other liquid in the well(s) that could allow the positive control well(s) to begin reacting without the addition of the patient liquid sample(s) to be tested.
4. The final step 3-4′ is to remove the strips from the freeze dryer in a low humidity or inert gas environment and package them in a sealed pouch. In an embodiment a desiccant is placed in the pouch to ensure that no moisture reaches the well(s). In a preferred embodiment, the packaging and sealing of the strip into a moisture proof container would include flushing the strip with an inert gas (e.g. nitrogen or argon) at a pressure below 1 atm and sealing the package (e.g. heat sealing to a cover (e.g. plastic, mylar or foil) such that the sealed cover is slightly pushed in. This feature allows one to easily identify a package exposed to air.

While It is also envisioned that the positive control well(s) could be prepared with only the end stage substrates SC3 133 and SC1 131 needed for activation of the signaling tethered enzyme TE2 130 of FIG. 1 or other substrates in coupled reaction, there is advantage in using a positive control with a form of the biomarker being assayed as it will reduce variations due to temperature as the test and positive control wells 552T and 552P of FIG. 4E will be impacted in the same way

Byproducts of hemolysis can adversely affect the measurements of a biomarker in a patient sample. There are two primary causes, one is the change in color to a pink or red for higher levels of hemolysis, that can affect transmission/absorbance of light. The other is the biological activity of components coming out of damaged blood cells. For the NSE-FAST assay, the main interfering component coming from hemolyzed red blood cells (RBCs) is the enzyme Adenylate Kinase (ADK), which in the forward reaction acts on molecules of ADP to produce ATP. If not part of a pre-treatment step A0-101 of FIGS. 1 and 2, to reduce ADK interference effects, for the NSE-FA assay, coming from hemolysis, a preferred embodiment of the Mixture 13′ would include the addition of an ADK inhibitor. This will then also have the ADK inhibitor be included in the subsequent mixture 14′.

Examples of ADK inhibitors that may be used in embodiments include but are not limited to:
P1,P5-di(adenosine 5′) pentaphosphate (Ap5A)
Actinomycin D
Diadenosine tetraphosphate (Ap4A)
Diadenosine triphosphate (Ap3A)

These changes to the mixture 13′ composition will allow for meaningful results from blood samples with hemolysis levels up to 3 on the CDC Hemolysis Reference Palette (within the range of 20 to 50mg/dL hemoglobin).

FIG. 8 shows a top view of a preferred embodiment of a production module 480 being a standard 96-well, 8-strip holder 485 with eight empty 12-well strips 411 though 418. The benefit of the holder 485 is that it allows easy handing during production of eight strips at a time and similarly is designed to be used in standard medical fluid handling devices for production, testing and also in the final use with one or more strips in a medical testing lab. It is also envisioned that embodiments of the present invention can function with as few as 3 wells (i.e., a test well, a positive control well and a negative control well). Thus while the holder 485 shows eight 12-well strips, it could instead hold twelve 8-well strips, sixteen 6-well strips or thirty-two 3-well strips.

Note that strips can be manufactured in various configurations (e.g., one or more negative control wells in a row, followed by one or more test wells in a row, followed by one or more positive control wells in a row; or one or more test wells, followed by one or more negative controls, followed by one or more positive controls, etc. ,). FIG. 9A is a top view of the production module 480A with holder 485 with the eight strips 411A-418A after layer 461 of FIG. 4C has been deposited into the test and negative control wells. Here, for illustrative purposes, we show a pattern of one positive control well, followed by one test well, followed by one negative control well, with this triplet pattern repeating four times in a 12-well strip. Specifically in strip 411A, Layer 461 of FIG. 4C is shown in the four test wells 411AT1, 411AT2, 411AT3 and 411AT4 and four negative control wells 411AN1, 411AN2, 411AN3 and 411AN4. The positive control wells 411AP1, 411AP2, 411AP3 and 411AP4 are still empty at this stage. There are similar deposits in the test and negative control wells for the other 7 strips 412A through 418A.

FIG. 9B is a top view of the production module 480B with holder 485 with the eight strips 411B-418B after completion of Sub-Process 1 of FIG. 7 where Layer 462 of FIG. 4C has been deposited into the positive control wells. Specifically in strip 411A, Layer 462 of FIG. 4C is shown in the four positive control wells 411AP1, 411AP2, 411AP3 and 411AP4. There are similar deposits in the positive control wells for the other seven strips 412B through 418B. Up to this point, the process can be performed at temperatures between 4 degrees C up to room temperature.

FIG. 9B′ is a top view of the production module 480B′ with holder 485 with eight strips 411B-418B as it would be after step 2-2 of Sub-Process 2 of FIG. 7 where the production module 480B is placed in a rectangular depression 495 in the top of the freezer block 490 after the

freezer block 490 has been removed from a freezer (not shown) after step 2-1 of FIG. 7. In this configuration, the production module 480B′ with the holder 485 and freezer block 490 is re-inserted into a freezer for Y minutes at E degrees C as shown in step 2-3 of FIG. 7.

FIG. 9C is a top view of the production module 480C with holder 485 with eight strips 411C-418C on the freezer block 490 as it would be after completed step 3-1 of Sub-Process 3 of FIG. 7 where the production module 480B′ including the freezer block 490 has been removed from the freezer completing Sub-Process 2 and has then received the final Layer 463 of FIG. 4C where mixture 15 of FIG. 7 has been deposited into the positive control and test wells. Specifically in strip 411C, Layer 463 is shown in the four positive control wells 411CP1, 411CP2, 411CP3 and 411CP4 and four test wells 411CT1, 411CT2, 411CT3 and 411CT4. The four negative control wells are skipped in this step. In this configuration, the production module 480C with freezer block 490 is re-inserted again into the freezer for Z minutes at F degrees C as shown by step 3-2 of FIG. 7.

FIG. 9D is a top view of the configuration of the completed production module 480D after steps 3-2 and 3-3 of FIG. 7, now removed from the freezer block 490 of FIG. 9C and cycled through a freeze dryer to lyophilize the contents of the wells. In this configuration, the holder 485 and eight strips 411D-418D have been removed from the freezer block 490 in a low humidity environment

and are ready for removal from the holder 485 and insertion and sealing into light and moisture proof pouches. In a preferred embodiment a desiccant pack/module is inserted into the pouch enclosing the freeze-dried strip to ensure no moisture reaches the positive control wells, which will begin to react once wet.

In this configuration, there are four positive control wells, four test wells and four negative control wells in each strip ready for use with samples from a patient. For example, for strip 411D, the wells 411DP1, 411DP2, 411DP3 and 411DP4 are positive control wells, the wells 411DT1, 411DT2, 411DT3 and 411DT4 are test wells and the wells 411DN1, 411DN2, 411DN3 and 411DN4 are negative control wells.

The embodiment of FIG. 9D shows the 12-well configuration of PTNPTNPTNPTN where P is a positive control well, T is a test well and N is a negative control well. It is also envisioned that other embodiments of different orders of wells would be viable including NNNNTTTTPPPP, TTTTPPPPNNNN, PPPPTTTTNNNN as well as TTPPNNTTPPNN etc. Also, if 3, 6 or 9 wells are produced, layout embodiments are envisioned such as NNTTPP, PPTTNN, PTNPTN, NNNTTTPPP, PPPTTTNNN, PTNPTNPTN etc. It is also envisioned that using the test well without either or both positive and negative controls could be functional.

FIG. 9E shows a top view of a preferred embodiment of the production arrangement 590 having a 96 well holder 595 with a strip package 585 having 6 slots 586A, 586B, 596C, 586D, 586E and 586F into which each has a strip 519A, 519B, 519C, 519D, 519E and 519F respectively shown in preparation for the freeze drying step 3 of FIGS. 7 and 3′ of FIG. 7′.

Each 8 well strip 519A-519F has three positive control wells 511P, 513P and 515P, three test wells 512T, 514T and 516T and two negative control wells 517N and 518. In an embodiment the well 518 can alternately be a well to measure the level of hemolysis in the sample. The configuration shown 590 is ideal for use with a robotic liquid handler such as the Hamilton Microlab NIMBUS.

A preferred embodiment of well 518 has it acting as a Hemolysis Quantification Well (HQW). An HQW 518 will generate a signal that is proportional to the level of hemolysis when compared with the negative control well such as the well 517N. The present invention envisions three embodiments for HQW 518. These include
1. a well 518 similar to the negative control well that does not include an ADK inhibitor in the other wells including the negative control well 517N;
2. a well 518 with luciferase as the only enzyme with no potassium in the mixture; or
3. a well 518 with luminal and a peroxide enzyme that generates H₂O₂ to react with the hemoglobin to generate luminescence.

Following freeze drying, it is envisioned that a cover can be sealed to the 6-strip package 585 in the presence of an inert, dry gas (e.g., nitrogen or argon), to prevent moisture from reaching the wells as such would cause premature reaction in the positive control wells 511P, 513P and 515P. The sealing process includes but is not limited to heat sealing and adhesive sealing. A preferred embodiment of the sealing process is to be accomplished in an inert atmosphere at less than 1 atm so that the top of the package (not shown) will show depression. If the depression is not present, the seal has been compromised and the strip should not be used.

After sealing, a simple fixture can be used to separate the sealed package 585 into 6 separate strips. Alternatively, the package may be perforated to allow each strip to be torn off one at a time.

The embodiment of FIG. 9E shows the 8-well configuration of PTPTPTNN where P is a positive control well, T is a test well and N is a negative control well. It is also envisioned that other embodiments of different orders of wells would be viable. A preferred embodiment of the 8-well layout (from 511-518) is NNTTTPPP. This has the advantage that it minimizes potential signal crosstalk between the dimmest wells (negative control) and the brightest wells—the positive controls. It also puts the wells being analyzed close together to avoid potential spread of the luminescence signals due to the time delays in pipetting and sequentially measuring the light from the wells. In addition, if the person pipetting the sample accidentally dips the pipette into the wells it limits potential contamination of carrying a substrate or positive control biomarker into the test or negative control wells. This technique applies to any number of wells in a present invention embodiment.

FIG. 10A is a schematic view of the freezer block 490 with depression 495 placed on top of an insulating pad 500 with pedestals 502 to limit the heat flow from the insulating pad to the freezer block 490 so it will remain at a cold temperature during Sub-Process 3 of FIG. 7 when layer 463 of FIG. 4C is added. This will ensure that layer 462 of FIG. 4C of the positive control wells will remain frozen and not start the reaction as the liquid of layer 463 is placed on top of layer 462. The freezer block 490 includes slots 496 and 497 to allow insertion of a handle 499 of FIG. 10B to facilitate moving the freezer block into and out of the freezer and freeze dryer during Sub-Process 2 and Sub-Process 3 of FIG. 7. It is envisioned that other embodiments of the handle could work including indentations 496 and 497 on the side of the freezer block 490 to allow a separate tongs to lift from above or a permanent rotatable handle that would rotate to the side lying flat during production and be rotatable to a position to allow the block 490 to be grabbed with gloves.

FIG. 10B is a schematic view of the Freezer block 490′ with the handle 499 inserted into the slots 496 and 497. Ideally the handle 499 is made of a material with low thermal conductivity such as wood or plastic. This is of particular importance if a very low temperature freezer (e.g. a −80° C. freezer) is used in Sub-Processes 2 and 3 of FIG. 7.

In a preferred embodiment where the freezer block 490 is used with a liquid-dispensing robot like the Hamilton NIMBUS or Opentrons Flex OT-2 Workstation, the insulating pad 500 would be sized to fit in the opening for a standard 96 well plate and fit into a recess (not shown) in the bottom of the freezer block 490 to ensure alignment of the recess 495 with the recess in the base of the robot that ensures accurate pipetting of fluids into the wells shown in FIGS. 9A through 9C.

It is also envisioned that a larger freezer block designed to hold multiple production modules 480 of FIG. 8 could be produced to facilitate production of multiple sets of eight 12-well strips (or multiple sets of sixteen 6- well strips etc.) during each stage of the Sub-Processes 1 and 3 of FIG. 7

FIG. 11A is a graph showing an example of the luminescence data over time from a 12-well strip over ten minutes. Each dot represents the amplitude of the luminescence from one of the 12 wells being the number of photons detected from the well by a detection device (e.g., a plate reader) during the half second measurement period used to sequentially measure each well. For this example, the TET coupled enzyme assay strip (e.g., the strip 400 of FIG. 4A) has four sets of three wells along its length with each set having a test well (e.g., the well 452T in FIG. 4C), a positive control well (e.g., the well 452P of FIG. 4C), and a negative control well (e.g., the well 452N of FIG. 4C). When placed into a standard plate reader such as the Tecan Infinite 200 PRO, each of the strip's 12 wells has its luminescence measured in turn repeatedly with all 12 wells read every 10 seconds for a 10 minute duration period by the light detector (e.g., a photomultiplier tube) in the plate reader.

Each dot represents the number of photons detected over the pre-set time period (e.g., 0.5 seconds) and with different shades of dots for each of the 12 wells as follows:
811L, 812L, 813L and 814L representing the luminescence from each of the four positive control wells,
821L, 822L, 823L and 824L representing the luminescence from each of the four test wells,
831L, 832L, 833L and 834L representing the luminescence from each of the four negative control wells.

The kinetics of the luminescence produced by the coupled enzyme assays of the present invention embodiments are such that the activity of the enzyme in the sample (e.g., NSE, ALT or AST) is designed to be approximately proportional to the rate of the luminescence photon production (given as LU) per second. The rate of the light output (luminescence) is determined by the slope of each of the N, T, and P traces, with units of Luminescence Units (LU) per Second (LU/Sec).

Plate readers (e.g., the TECAN Infinite 200 Pro) include software that can calculate a linear slope from the data collected for each well's luminescence over a measurement portion of the duration period (e.g., 10 minutes in FIG. 11A). It is envisioned that this measurement period can be as short as 30 seconds and as long as 30 minutes with a preferred embodiment being 2-5 minutes. In a preferred embodiment, one would start the measurement period with an optional brief, pre-determined delay after the start of the assay, which commences upon addition of the fluid sample to the test, positive and negative control wells and insertion into the plate reader. For example, a 2 minute measurement period could begin 30 seconds after the start of the duration period.

FIG. 11A shows the lines of best fit for the 12 slopes calculated for each of the 12 wells and three average slopes as:
811S, 812S, 813S and 814S representing the luminescence slopes from each of the 4 positive control wells,
821S, 822S, 823S and 824S representing the luminescence slopes from each of the 4 test wells,
831S, 832S, 833S and 834S representing the luminescence slopes from each of the 4 negative control wells.
line representing the average slope for the four positive control wells 815(SP),
line representing the average slope for the four test wells 825(ST) and
line representing the average slope for the four negative control wells 835(SN).

In a preferred embodiment the average slopes 815(SP), 825(ST) and 835(SN) are used by the plate reader to calculate the relative activity of the enzyme (e.g., NSE-FA) in the sample.

To understand how this works one begins with the contributions to the luminescence for each type of well. It is also important to note that the rate of the enzymatic activity in the coupled enzyme reaction of embodiments of the present invention is approximately proportional to the slope of the luminescence curve during a period of M minutes of the reaction before saturation effects occur. In embodiments, M may be between 1 and 30 minutes that optionally may start after a short delay. While the total kinetics of a coupled enzyme assay like that of the present invention are influenced by the various rates of activities of each enzyme, and subject to internal influences such as changing concentrations of reaction products, substrates and cofactors, and subject to external influences such as temperature, for the sake of simplicity here, the methods described herein are envisioned as a viable technique to reproducibly determine the activity of the enzyme biomarker E1 of FIG. 1.

Specifically:
1. Luminescence from a negative control well will be produced from any molecule in the blood that could activate either tethered enzyme in the coupled enzyme reaction or react with luciferin. As the initial substrate for the biomarker enzyme being assayed is missing in the negative control, the luminescence data seen from the negative control wells 831L, 832L, 833L, and 834L will be the base luminescence of the test and positive control wells. If this is too high, it may signal an error condition that would require a re-test, for example. In embodiments of the present invention, means to identify error conditions from the negative control data are envisioned.
2. Luminescence from a test well will equal that of luminescence from a negative control well plus the luminescence produced by the reaction of the substrates SA1 and SA2 with the active Enzyme E1 of FIG. 1. Thus, the normalized (Real) value of the activity of the Enzyme E1 sample pipetted into a test well(s) S_{TestReal} (S_{TR}) can be calculated by subtracting the luminescence slope SN of the negative control well(s) from S_{T} of the test well(s).
3. Luminescence from a positive control well will include the luminescence from a test well (2 above) plus the luminescence produced by the additional enzyme pre-seeded in the positive-control well in layer 462 of FIG. 4C or the enzyme pre-seeded in the second layer 563 of FIG. 4E. Thus, the normalized (Real) value of the activity of the pre-seeded enzyme in layer 462 of FIG. 4C or 563 of FIG. 4E S_{PositiveReal} (S_{PR}) can be calculated by subtracting the luminescence slope ST of the test wells from luminescence slope S_{P} of the positive control well(s).

Mathematically, the Real luminescence slope S_{PR} defined as the fraction of luminescence from only pre-seeded enzyme in the positive control well(s) (e.g., enolase for NSE-FA) can be calculated as:

$${S}_{PR} = {S}_{P} - {S}_{T}$$

and the Real luminescence slope S_{TR} from the NSE-FA in the patient sample from the test wells defined as the fraction of luminescence from only the biomarker enzyme E1 of FIG. 1 in the sample added to the test well(s) can be calculated as:

$${S}_{TR} = {S}_{T} - {S}_{N}$$

The values of S_{PR} and S_{TR} provide a means to not only measure the activity of the enzyme E1 of FIG. 1 in the patient sample (e.g., NSE-FA), but also provide a technique that can be used to set a threshold for a normal range for such activity (e.g., defining significant brain injury for the NSE-FA coupled enzyme assay).

In a preferred embodiment, an Activity Level can be calculated where the Activity Level (A_{P}) is STR expressed as percentage of S_{PR} i.e.

$${A}_{P} = 100 \times {S}_{TR} / {S}_{PR}$$

In terms of the slopes from the measured luminescence traces, this would be

$${A}_{P} = 100 \times ( {S}_{T} - {S}_{N} ) / ( {S}_{P} - {S}_{T} )$$

It is also envisioned that a fraction A_{F} may be used where A_{F}=S_{TR}/S_{PR}.

In another embodiment, the positive control and test well luminescence slopes may be normalized to the negative control only and the negative control normalized activity may be represented as a percentage or fraction as:

$${A}_{N} = 100 \times ( {S}_{T} - {S}_{N} ) / ( {S}_{P} - {S}_{N} ) , or \\ {A}_{F} = ( {S}_{T} - {S}_{N} ) / ( {S}_{P} - {S}_{N} )$$

Whether a fraction or percentage, these normalized values of activity can be compared to a normal range of activity defined by thresholds having a lower bound A_{TL} and upper bound A_{TU} where detection of abnormal biomarker levels would be levels of activity below A_{TL} or above A_{TU}.

For detection of ischemic stroke by measurement of NSE-FA, a lower threshold would not have clinical utility, and values below or equal to a single threshold level would be considered normal, whereas those greater than the normal cut-off would be considered abnormal.

In other embodiments, the 12 slopes may be used to reduce variance by taking the median of the slopes instead of the average or by eliminating the high and low and averaging the two other slopes to get the average slopes SP, ST and SN.

In still other embodiments, the 12 slopes may be used to reduce variance by removing any one slope that differs by more than some percentage (e.g., 5%) from the other three values.

It is envisioned that a term such as “amplitude” may be used in preparing data such as seen in FIG. 11A, to represent the number of photons detected over a half second by the reader used; however, it is also envisioned that other measurements of the amount of luminescence from a well or zone including:
1. the voltage at a specific time or the average voltage from a photodiode, or
2. a counted number of photons by a photo detector tube in a luminescence reader, or
3. the output from a charge-coupled device at a single point in time or averaged over to pre-set time period.

While FIG. 11A uses average slope to calculate biomarker levels, it is envisioned that other measurements such as Area Under the Curve (AUC) shown in FIG. 11B or the average luminescence at a single point of time shown in FIG. 11C can provide a basis for alternative algorithms to quantify biomarker levels and establish clinically useful thresholds

FIG. 11B is a graph showing the data for an alternate mathematical embodiment using the Area Under the Curve (AUC_{P}, for the positive control wells, AUC_{T} for the test wells and AUC_{N} for the negative control wells) to measure luminescence data from a coupled enzyme reaction. Similar subtractive methods or other algorithms may be applied to these values over a preset duration period (e.g., ˜10 minutes in FIG. 11B) to calculate the activity of the biomarker in the patient samples.

FIG. 11C is a graph showing the data for an alternate mathematical embodiment to measure luminescence data from a coupled enzyme reaction using an average value of the luminescence (P_{P} for the positive control wells, P_{T} for the test wells and P_{N} for the negative control wells) at a single point in time.

FIGS. 11B and 11C are alternatives to the scheme using slope described for FIG. 11A. These alternatives may be preferable for various embodiments, such as where the biomarker is a substrate and not an enzyme. These alternate embodiments of the present invention would include algorithms for measuring amounts of biomarker in a patient sample such as:
Measurement based on the area under the curves (AUC) as shown in FIG. 11B. Here calculations would be based on the values of the areas under the curves for the positive controls AUC_{P}, test wells AUC_{T} and negative control wells AUC_{N}. FIG. 11B shows 600 seconds but a longer or shorter time may be utilized. The calculation of the Real luminescence from the sample pipetted into the test wells AUC_{TR} and Real luminescence from the biomarker pre-seeded in the positive control wells AUC_{PR} can be calculated similarly to that shown in FIG. 11A where: AUC_{TR}=AUC_{T}−AUC_{N} and AUC_{PR}=AUC_{P}−AUC_{T}. The measured biomarker level can then be assessed as AUC_{TR} as a percentage or fraction of AUC_{PR}.
Measurement based on the peak luminescence over a pre-set time period as shown in FIG. 11C (in this example, for a 10 minute (600 second) time period). Here a similar algorithm to that used for slopes on FIG. 11A or area under the curve for FIG. 11B can be implemented. For example, the calculation of the Real luminescence from the sample pipetted into the test wells P_{TR} and Real luminescence from the biomarker pre-seeded in the positive wells P_{PR} can be calculated similarly to that shown in FIG. 11A where: P_{TR}=P_{T}−P_{N} and P_{PR}=P_{P}−P_{T}. The measured biomarker level can then be assessed as P_{TR} as a percentage or fraction of P_{PR}.

Other viable envisioned embodiments would include using:
The average of multiple activity measurements based on slope taken at a multiplicity of time points.
Measurement of the second derivative of the luminescence data curves.
Measurement of time to a peak value of luminescence.
Various combinations of the above (for example Peak Luminescence X Slope)

FIG. 11D shows a graph 850 of test 853, high-reference/positive control 851 and low reference/negative control 852 luminescence curves for an example of the light produced by a Coupled Tethered Enzyme Luminescence Assay (CTELA). The horizontal axis is time in seconds. Many of the embodiments of the present invention CTELA utilize a positive control or reference that allows analytics for the light output from the CTELA to determine the amount of biomarker or analyte in the sample. As previously discussed, a preferred embodiment of the present invention utilized the slopes of the luminescence curves to calculate the amount of biomarker/analyte in the sample. The example of FIG. 11D, shows red dashed lines to represent the slopes 854, 855, and 856 respectively of the of the luminescence curves of the positive control 851, negative control 852 and test 853 curves.

A preferred embodiment of the present invention CTELA uses different time periods for the measurement of the slopes 854, 855 and 856. Specifically in this example, the dashed boxes 857, 858 and 859 represent the time periods in which the slopes 854, 855 and 856 are calculated.

As previously described with respect to FIG. 4C, the materials in the positive control IVD well(s) are freeze dried in layers to prevent mixing or reaction of the substrate in layer 463 with the mixture including a preset amount of analyte in the layer 463. One benefit of using different time periods to measure the slope of the positive control as compared to the test well is to compensate for an additional delay required to rehydrate the freeze-dried materials in the positive control well 452P to allow the pre-seeded analyte (e.g., NSE or enolase) in the layer 462 to react with the substrate (e.g. 2-PG) in the layer 463 when liquid in the sample is placed in the well 452P.

This differs from the reaction in the test well 452T where all of the analyte (e.g. NSE) is hydrated and active in the liquid pipetted into the well 452T and can immediately act upon the substrate (e.g., 2-PG) in the layer 463 causing the coupled enzyme reaction to start sooner.

FIG. 11D shows where the test well slope 856 is measured over a time period 859 that starts earlier than the measurement of the positive control/reference slope 854 measured over the time period represented by the box 857.

The time period represented by the box 858 used for measuring the negative control/reference slope 855 may be the same or different than either the time periods 857 and 859 used for the positive control 851 or test curves 853.

The three luminescence curves 851, 852 and 853 may represent a single well of an IVD or reaction zone of a lateral flow assay or may be the average or the sum of the luminescence from multiple wells or reaction zones.

These embodiments of the present invention shown here utilize a different time period for the calculation of slope of the positive control luminescence 851 vs. that of the test luminescence curve 853. In a preferred embodiment, the start of the time period 857 for the positive control/reference would be delayed by a delay Q 860 from the start of the time period 859 for calculating the slope of test well luminescence curve 853.

While the time periods represented by the positive control box 857 and test box 859 are of similar duration, it is envisioned that different durations may be used.

While FIG. 11D shows the use of different time periods for measurement of slope of the luminescence curves 851, 852 and 853, it is also envisioned that the other techniques shown in FIGS. 11B and 11C such as area under the curve or value at a specific time may also take advantage of the present invention using different time periods and durations for analysis of luminescence curves.

In a preferred embodiment of the present invention, the test luminescence curve would have its slope measured over a time period of at least 15 seconds starting within 15 seconds of the start of the luminescence data represented by time=0 on the horizontal axis of FIG. 11D.

In a preferred embodiment of the present invention, the positive control/reference luminescence curve would have its slope measured over a time period of at least 15 seconds starting at least 10 seconds after the start of the slope measurement of luminescence data for the test curve 853.

An example of such a preferred embodiment would be the one minute test shown in FIG. 11D where the slope 856 of the test curve 853 is measured during the first 30 seconds and the slope 854 of the positive control/reference curve 851 is measured during the second 30 seconds.

The kinetics for the CTELA used to detect/quantify any specific biomarker/analyte or assay implementation may be different. The present invention also includes the use of machine learning artificial intelligence to optimize the choices of start time and duration of the time periods used for calculations as described above. For example, for best detection of nervous system injury by the measurement of NSE in a sample, one could take hundreds or thousands of measurements of brain injury and non-brain injury measurements with the knowledge of which is which to optimize the threshold used to define/identify brain injury, or maximize differentiation between brain injury and non-brain injury results, with a broadened dynamic range potentially providing additional clinical utility (e.g., volume of infarct, prognostic information).

While the embodiment for FIG. 11D shows the measurement period 857 for the positive control curve 851 to start later than the measurement period 859 for the test well curve 853, it is envisioned that some embodiments can use different time periods that start together or have 857 start before 859.

FIGS. 12A, 12B, 12C and 12D show an embodiment of the top view of lateral flow blood separation filter paper embodiments of the present invention that would be applicable to a Point-of-Care (PoC) or home use assay using the present invention tethered enzyme technology. The configuration shown would be applicable to embodiments of assays for NSE-FA or liver enzyme ALT and AST activity as shown in FIGS. 1, 2, 3A, 3B and 3C.

FIG. 12A is a top view showing the blood separation paper strip set 900 with negative control strip 911, test strip 912 and positive control strip 913 as they might be configured before being used in a PoC diagnostic assay.

A preferred embodiment of the strip set 900 would have well reaction zones 961 and 962 of strips 911 and 912 including components similar to layer 461 of FIG. 4C, and zone 963 of positive control strip 913 including the components similar to layer 462 of FIG. 4C including a preset amount of the assayed biomarker. The band 921 of strips 912 and 913 would have components similar to layer 463 of FIG. 4C including the substrate on which the enzyme biomarker works (for example, 2-PG for the NSE-FA coupled enzyme assay shown in FIGS. 3A and 3B). Fluid input zone 919 in strips 911, 912 and 913 shows the location where whole blood is placed. In a preferred embodiment the fluid input zone 919 may include or be connected to a blood absorbing reservoir or physical barrier that can help prevent overflow if excess blood is placed onto the zone 919.

In preferred embodiments, the entire strip may be sealed or coated except for the fluid input zones 919. Examples of such coatings include plastic and paraffin. A small air vent (not shown) could be added below the zones 925 to ensure full flow of the plasma into and past the reaction zones 961, 962 and 963. It is envisioned that once the liquid blood/plasma is fully saturated in the paper strip it will stop flowing with plasma in the reaction zones 961, 962 and 963 each having approximately the same volume of plasma that can react to provide luminescence.

In an embodiment, the flow extension zones 925 of strips 911, 912 and 913 allow excess plasma to flow beyond the reaction zones 961-963, ensuring that each reaction zone has a standard and saturating volume that fills the paper in that zone, but which does not pool in excess. Other embodiments are envisioned that have the N, T, and P reaction on a single lateral flow paper, separated by hydrophobic separators such as ink or wax based. Rather than side-by-side, another embodiment might have the 3 lateral flow papers stacked over each other, with separation layer including photodiodes between the papers.

FIG. 12B is a top view showing the strips 900′ at a time of several seconds after a patient's blood has been placed in the fluid input zone 919 of the strip set 900. It shows the blood in area 950 of the test strips 911′, 912′ and 913′. As the liquid flows away from the area 950, blood cells and cell fragments (e.g., platelets) are retained within the areas 950-951 and the remaining plasma 952 continues to flow down the strips 911′, 912′ and 913′. As shown the plasma has just reached the bands 921.

FIG. 12C is a top view showing the assay strips 900″ as the plasma 952 has now filled the reaction zones 961, 962 and 963 and is filling the flow extension zones 925″ of the strips 911″, 912″ and 913″ respectively. As shown,
the plasma 952 of the negative control strip reached the negative control reaction zone 961;
the plasma 952 of the reaction test strip has mixed with the band 921″ bringing the reaction components and any products formed within the band 921″ into the reaction test zone 962; and
the plasma 952 of the positive control strip has mixed with the band 921″ bringing the components and any products formed found in the band 921″ into the reaction test zone 963.

The reaction zones 961, 962 and 963 will now produce luminescence that can be measured photometrically to provide the luminescence data such as that shown in FIG. 11A from which the activity of the enzyme E1 103 of FIG. 1 can be determined.

Examples for NSE-FA, ALT and AST of this preferred embodiment would have the following elements of FIG. 2 in the zones and bands of the strip set 900 as follows:

For NSE-FA, zones 961 and 962 would include the tethered enzymes Pyruvate Kinase (TET-PK) 303, tethered Luciferase (TET-Luciferase) 305 and the compounds ADP 302 and Luciferin 304 of FIG. 3A. The zone 963 would include the components in band 961 (or 962) plus a preset amount of enolase. In a preferred embodiment, the enolase would be tethered. The band 921 would include the substrate 2-PG 301 of FIG. 3A.

For ALT, zones 961 and 962 would include the tethered enzymes Glutamate Oxidase (TET-Glut-OX) and tethered Horse Radish Peroxidase (TET-HRP), and the compound Luminol shown in FIG. 2. The zone 963 would include the components in band 961 (or 962) plus a preset amount of the liver enzyme ALT. In a preferred embodiment, the ALT in zone 963 would be tethered. The band 921 would include the substrates α-ketoglutarate and L-Alaninine shown in FIG. 2.

For AST, zones 961 and 962 would include the tethered enzymes Glutamate Oxidase (TET-Glut-OX) and tethered Horse Radish Peroxidase (TET-HRP), and the compound Luminol shown in FIG. 2. The zone 963 would include the components in band 961 (or 962) plus a preset amount of the liver enzyme AST. In a preferred embodiment, the AST in zone 963 would be tethered. The band 921 would include the substrates α-ketoglutarate and L-Aspartate shown in FIG. 2.

In this embodiment pre-treatment of the blood or plasma with uricase (tethered or not) and uric acid is necessary for the ALT and AST luminescence coupled enzyme assays of the present invention. For liver enzyme use with the configuration of the strip set 900 of FIG. 12A, the blood would need be pre-treated before it is placed onto the strips 911, 912 and 913. In a preferred embodiment this pre-treatment uses freeze dried tethered uricase and uric acid placed into the blood collection vial from which blood is added to the strip set 900. This may be done by freeze drying the uric acid and uricase onto the inside surface of the vial or separately introducing them into the vial, for example by adding a tablet, or powder to the vial.

While it is possible to put all the components into the reaction zones 961, 962 and 963 using layering such as shown in FIG. 4C, an advantage of this preferred embodiment of the PoC assay is that the substrates with which the biomarker reacts in the band 921 are separated from the reaction zones 961, 962 and 963.

In embodiments of the present invention PoC coupled enzyme assay, it is envisioned that a single input of blood could be configured with the fluid input zones 919 of the negative control strip 911, test strip 912 and positive control strip 913 interconnected so that blood placed in the interconnected zone 919 would flow and be filtered into 3 or more blood filter/assay strips or channels such as the strips 900. Examples of this concept are shown in FIGS. 13A, 13B and 14.

For clarification, the term band such as the bands 921 of FIG. 12A refers to a collection of components meant to be picked up by fluid (e.g., plasma) flowing down the strips 912 and 913 such that those components are carried into the reaction zones 962 and 963 where they participate in any reaction producing assay luminescence. The reaction zones 961, 962 and 963 are the areas of the strips 911, 912 and 913 monitored by photon detection mechanisms adapted to measure the amplitude of the luminescence reaction occurring in each reaction zone.

FIG. 12D is a top view showing an alternate embodiment of the lateral flow blood separation assay paper strip set 950 with negative control strip 951, test strip 952 and positive control strip 953. In this embodiment, this negative control strip 951 is the same as the negative control strip 911 of FIG. 12A and the test strip 952 is the same as the test strip 912 of FIG. 12A. Specifically, the zone 971 is the same as the zone 961 of FIG. 12A, the zone 972 is the same as the zone 962 of FIG. 12 A, the band 974 is the same as the band 921 of FIG. 12A, and the area 975 is the same as the area 925 of FIG. 12A.

While the band 974 of the positive control strip 953 is the same as the band 921 of FIG. 12A, the positive control strip 953 differs in that zone 973 has the same components as the zones 971 and 972 and does not include a preset amount of biomarker as does the zone 963 of FIG. 12A. In this embodiment, the preset amount of biomarker is placed into band 922 that will release the biomarker into the flowing plasma as it flows down strip 953 similar to the flow of plasma in FIG. 12 B and 12C.

FIG. 12E is a top view showing the blood separation paper strip set 1200 for both liver enzymes ALT and AST with negative control strip 1211, ALT test strip 1212, ALT positive control strip 1213, AST test strip 1214 and AST positive control strip 1215 as they would be configured before being used in a PoC diagnostic assay.

The zones 1261, 1262 and 1264 include the tethered enzymes (see FIG. 2) TET-Glut-Ox, TET-HRP as well as Luminol for the ALT/AST liver tests. Zone 1263 also includes the pre-set amount of ALT (tethered or not) as the positive control for the ALT assay. Zone 1265 also includes a pre-set amount of AST (tethered or not) as the positive control for the AST assay.

The bands 1230 of strips 1212 and 1213 include α-ketoglutarate and L-Alanine, the substrates with which ALT reacts. The bands 1240 of strips 1214 and 1215 include α-ketoglutarate and L-Aspartate, the substrates with which AST reacts.

It is also envisioned that in embodiments, the positive control pre-set amounts can be in a separate band like the band 922 of FIG. 12D instead of in the zones 1263 and 1265 of FIG. 12E.

The embodiments of ALT and AST assays in FIG. 12E do not require separate pre-treatment of the blood in a separate vial but include the bands 1220 having uric acid and uricase (tethered or not) that will provide the pre-treatment as the blood and then plasma flow along the strips 1211 through 1215 so that the treatment will be completed before the plasma reaches the zones 1261 through 1265.

While the preferred embodiment of positive controls uses a preset amount of the biomarker being assayed (or an analog such as enolase for NSE), it is envisioned that alternate positive control formulations that include pre-set amounts of substrates/intermediates produced in later portions of the coupled enzyme reaction would function well. For example, for the NSE-FA assay, having a pre-set amount of phosphoenolpyruvate (PEP, which is produced by NSE-FA) or ATP could serve a similar purpose rather than a preset amount of enolase.

It is envisioned that instead of the paper chromatography strips shown in FIGS. 12A through 12D, for the purposes of this specification, the present invention embodiments of fluid flow strips may include any fluid flow entity (with or without blood separation capability) selected from the group of:
chromatography paper,
microfluidic channels,
membranes,
matrices, or beads/particles/fibers
any fluid flow entity in an enclosed space that will facilitate passive fluid flow (e.g., as achieved through capillary action), or active flow (e.g., as achieved with a pump).

For the purposes of this specification, the term test strip may represent any of the above types of fluid flow entity with a test zone designed to measure a biomarker, a positive control strip may represent any fluid flow entity that has a pre-set amount of the biomarker to allow the assay to quantify the amount of biomarker or biomarker activity in the sample, and the term negative control strip represents any fluid flow entity that can serve as a negative control for the present invention assay.

FIG. 12F represents another preferred embodiment of the blood separation strips used for the present invention assay 990 with negative control strip 991, test strip 992 and Positive control strip 993. Here the substrates 2-PG for NSE-FA, α-ketoglutarate and L-Alanine for ALT and α-ketoglutarate and L-Aspartate for AST that are contained in separate bands in FIGS. 12A through 12E, are in this embodiment 990, included in the zones 982 of the test strip 992 and 983 of the positive control strip 993. None of the substrates now in zones 982 and 983 are placed in the zone 981 of the negative control strip 991. This design is similar to the strip 550 of FIG. 4E, where only the positive control well 552P gets a second layer 563 that includes a pre-set amount of an enolase for the NSE-FA assay and ALT or AST for their assays. Here the band 995 of the positive control strip 993 includes a pre-set amount of an enolase for the NSE-FA assay and ALT or AST for their assays.

The design of the assay 990 reduces the number of total bands to only one as compared to two bands 921 in the assay 900 of FIGS. 12A through 12C and three bands 974 and 922 of the assay 950 of FIG. 12D. It is also envisioned that an ATK inhibitor may be included into any of the zones 961, 962, 963, 971, 972, 973, 1261, 1262, 1263, 1264, 1265, 981,982 and 983 of FIGS. 12A-12F to reduce the impact of hemolysis on the assay. Alternately, the ATK inhibitor can be included in any of the bands or as a separate band in any of the embodiments shown.

FIG. 13A is a schematic view showing an embodiment of a TET assay card 1280 configured for insertion into a photodiode-based reader. The assay card 1280 has a main body 1281, handle 1282 and assay structure 1250 having a fluid input zone 1290, blood separation strip 1221 and plasma flow structures 1295, 1296, and 1297 with 1295 connected to the test zone 1291T, 1296 connected to the negative control zone 1291N and 1297 connected to the positive control zone 1291P.

The plasma flow structures 1295 and 1297 have substrate bands 1231 similar to the bands 921 of FIG. 12A and the bands 974 of FIG. 12D. The plasma flow structure 1297 also has a positive control band 1232 having a preset amount of the biomarker being assayed similar to the band 922 of the positive control strip 953 of FIG. 12D. In this embodiment, the materials in zone 1291T are similar to that of zone 972 of FIG. 12D, the contents of the zone 1291N are similar to that of zone 971 if FIG. 12D and the contents of zone 1291P are the same as the that of zone 973 of FIG. 12D.

In a preferred embodiment, the band 1232 is missing and like the embodiment of FIG. 12A, the pre-set amount of biomarker is included in zone 1211P similar to that of zone 963 of FIG. 12A.

In this preferred embodiment where the positive control zone 1211P includes a pre-set amount of biomarker, the operation of the card 1200 is similar to that shown with the strip sets 900′ and 900″ of FIGS. 12B and 12C where blood cells and cell fragments are restrained within the blood separation strip 1221 allowing plasma to flow down the plasma flow structures 1215, 1216 and 1217 picking up materials in the substrate bands 1231 (test and positive controls only) and flowing into the reaction zones 1211T, 1211N and 1211P respectively.

In use, the card 1200 would be removed from its sealed and light-proof pouch, blood from a finger- or heel-prick or point of care collection device would be placed into the fluid input zone 1210 and the card 1200 held by the handle 1202 would be placed into a photodiode detection device whereupon insertion would, in a preferred embodiment, activate or turn on the device that would then analyze the luminescence from the three reaction zones 1211T, 1211N and 1211P to provide a quantitative measurement of the biomarker and/or indicator of the test result as being one or more of: high, low, or normal (if upper and lower thresholds are applied); and/or negative (normal) or positive (abnormal) if a single threshold is applied.

For example, NSE-FA that exceeds a threshold would indicate significant acute brain injury that might be associated with a stroke or concussion. Note that the indicator could have various embodiments, including visual displays (e.g., text saying “high” versus “normal,” or one or more colored lights such as green for normal range values, versus red for high values), or an auditory indicator for values exceeding the threshold, or any combination thereof.

FIG. 13B is a top view showing an embodiment of a TET diagnostic PoC card layout 1300 where the fluid input zone 1320 is centrally located with 5 blood separation strips 1311, 1312, 1313, 1314 and 1315 leading outward to reaction zones 1301, 1302, 1303, 1304 and 1305 respectively. Substrate bands 1321, 1322, 1323 and 1324 can have components or function similarly to the bands 921 of FIG. 12A. For example, the light grey bands could be for ALT and the dark grey bands for AST.

Embodiments of the card 1300 could have any combination of test, positive control and negative control reaction zones; however, a preferred embodiment would have one negative control zone, one positive control zone and three test reaction zones.

In a preferred embodiment such as that for liver enzymes ALT and AST, one could have a configuration where the 5 strips would have similar function to the 5 strips of FIG. 12E with zone 1301 being the test zone for ALT, 1302 the positive control for ALT, 1303 being the test zone for AST, 1304 being the positive control zone for AST and 1305 being the negative control zone. This configuration would use the blood pre-treatment method in a collection vial of FIG. 3C. In this case, the components within bands 1321 and 1322 would be similar to band 1230 in FIG. 12E, and the components within bands 1323 and 1324 would be similar to band 1240 in FIG. 12E. An alternate embodiment would include an added band or area for pre-treatment in or near the fluid input zone 1320. Also as previously described, any of these embodiments may include an ADK inhibitor in the blood collection apparatus or in the blood deposit area such as 1320 or the bands 1321-1324 or zones 1301-1305.

FIG. 14 is a schematic view of an embodiment of a TET coupled enzyme assay test module 1400 where a blood sample volume 1450 is deposited into the upper cylinder 1420 with strip holder 1411 and 4 blood separation paper strips 1451, 1452, 1453 and not shown 1454 having fluid input zones 1481, 1482, 1483 and 1484 (not shown). The strip 1452 is the test strip with the substrate band 1462 and reaction zone 1472 similar to the strip 912 with band 921 and reaction zone 962 of FIG. 12A. In an embodiment, a valve 1404 with actuator 1406 would allow blood to be placed in the upper portion 1490 of the upper cylinder 1420 to control the start of blood flow into the strips 1451, 1452, 1453 and 1454 (not shown). In embodiments, a portion of the actuator 1406 lies outside of the upper cylinder 1420.

The strip 1451 is a negative control strip with reaction zone 1471 similar to the strip 911 with reaction zone 961 of FIG. 12A. Optical separation is important for an accurate reading of luminescence. To facilitate that, the test module 1400 includes optically opaque separators 1441 and 1442 with two not shown 1443 and 1444. The allows the module 1400 to be inserted into a photodiode-based optical reader such as that shown in FIG. 16 where at least one photodiode is aligned with each reaction zone e.g., 1471, 1472 etc., to have each photodiode accurately measure the luminescence from each zone and with electronic circuitry, that may include a microcomputer, analyze the results using an activity measurement calculation such as those described in association with FIGS. 11A, 11B and 11C.

The module 1400 can be used in several ways. In some embodiments it may be a stand-alone fully disposable device with an integrated reader with blood inserted from a syringe, vacuum collection tube, or other devices used to collect blood from venipuncture or a finger prick, or be set to be attached as a microtainer to a blood collection device such as the TASSO of Tasso, Inc.

In a preferred embodiment, the module 1400 can be designed to be inserted into a photodiode-based reader where only the module 1400 need be disposable.

Although four reaction zones 1471-1474 are described here, as few as one and as many as twenty zones or more may be used with a preferred embodiment of three or four zones.

The flow extension zones 1475, 1476, and not shown 1477 and 1478 perform the same function as the flow extension zones 925 of FIG. 12A.

It is also envisioned that each of the strips 1451, 1452, 1453 and 1454 except for the fluid input zones 1481, 1482, 1483 and 1484 could be coated with a sealing material, e.g., plastic or paraffin. This will cause the plasma to flow down each strip 1471-1474 and stop once the filter paper is fully saturated. A small air vent may also be added to the bottom of the sealed strips 1451, 1452, 1453 and 1454. Similar coatings are also applicable to the embodiments shown in FIGS. 13A, 13B, 13C, 15 and 16.

Also as previously described, any of these embodiments may include an ADK inhibitor in the blood collection apparatus or in the blood deposit area such as 1411 or the bands 1462 or zones 1471-1474.

FIG. 15 is a schematic view of an embodiment of a two-piece TASSO/TET assay system 1500 with a Photonic Luminescence Reader (PLR) 1550 and the TASSO blood collection device 1510 with initiation button 1512 and with the normal collection vial replaced by an embodiment of the TET coupled enzyme assay test module 1520 similar to the test module 1400 of FIG. 14 but with the addition of the alignment key 1525. The test module 1520 is designed to receive the blood collected by the TASSO device 1500, separate the cells from the plasma allowing the plasma to flow along the four strips 1521,1522, 1523 and 1524 (not shown) into the reaction zones 1531, 1532 and 1533 and 1534 (both not shown), picking up where needed chemicals in for example the substrate band 1542 of the test strip 1522.

The Photonic Luminescence Reader (PLR) 1550 with upper case 1552 includes an alignment female key slot 1582 as part of the generally cylindrical guide 1580. The guide 1580 has four attached photodiodes 1591, 1592, 1593 and not shown 1594 with cables 1595, 1596, 1597 and not shown 1598 to attach the photodiodes to electronic circuitry in the electronics module 1570. Each of the cables 1591-1594 has typically one or two wires each. The photodiodes 1591-1594 are attached to the cylindrical guide 1580 with at least the portion of the guide 1580 where the photodiodes 1591-1594 are attached being optically transparent. This can be accomplished by different embodiments including having a hole in the guide 1580, having the entire guide 1580 be transparent or in a preferred embodiment, having a transparent window under a portion of the photodiodes 1591, 1592, 1593 and not shown 1594. Embodiments with holes or windows may be preferred to prevent light leakage from one zone being detected by a photodiode aligned with another zone (i.e. crosstalk).

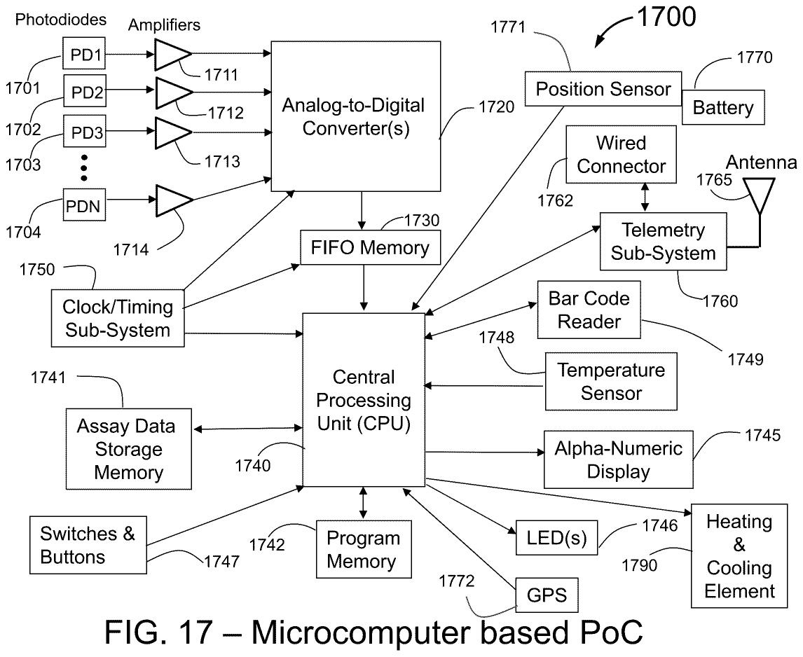

The PLR 1550 has an electronics module 1570 attached to the bottom of the upper case 1552 into which the cables 1595-1598 bring the signals from the photodiodes 1591-1594 to the electronic circuitry with an embodiment shown in FIG. 17. The outside of the PLR also includes a start button or switch 1572, a digital readout 1576 and an indicator LED 1574. The digital readout 1576 provides information on the quantitative measurement of the assay performed by the PLR 1550 with the LED 1574 being able to indicate one or more detection-related conditions. For example, the LED 1574 might be red/green/yellow LED where it would be green if detection of NSE-FA is below the threshold for brain injury and red if above. It might flash while the device is working and could go yellow for an error condition. As noted above, other indicators including auditory could be present in different embodiments. In an embodiment of the present invention module 1520, strip 1521 is the negative control strip, strip 1522 is the test strip and strip 1523 is the positive control strip. In an embodiment, the 4^{th} strip 1524 that is hidden behind the schematic view, can be one of the following:
An additional test strip
An additional positive or negative control strip
An additional hemolysis quantification strip
An additional biomarker assay test different from that of the test strip 1522, for example for the NSE-FA assay a test that includes an inhibitor for Neuron Specific Enolase and allow luminescence from other types of enolase in the sample. Another example for liver enzymes is to have strip 1522 be for ALT and strip 1524 for AST with a common negative control (or positive control) for comparison.

It is envisioned that multiple digital displays or LEDs might also be used with configurations that could include indication of power on, negative result, positive result, error condition, test working and/or numerical or alphanumerical displays. Embodiments of the electronics module 1570 having wireless or wired telemetry as shown in FIG. 17 is also envisioned. One embodiment of the method for using the system 1500 for detecting and measuring acute brain injury is as follows:
1. The TASSO device 1510 is removed from its package
2. The TET module 1520 is attached to the TASSO device 1510 in place of the normal blood vial container;
3. The TASSO is placed on the patient's arm, the central button 1512 is pressed to initiate blood collection;
4. After a pre-set time (e.g. 2-5 minutes) or when the blood fills the TET module 1520 to a marked level, the entire TASSO 1510 or just the module 1520 is removed from the patient and inserted into the PLR 1550 aligning the key 1525 of the module 1520 with the slot 1582 of the PLR 1550.
5. The start button 1572 is pressed and the electronic circuitry of the electronics module 1570 will collect luminescence data from the 4 photodiodes 1591-1594 for a pre-set period.
6. The electronics module 1570 will then calculate the enzymatic activity of the biomarker being assayed and show the result on the numerical display 1576. Embodiments of example calculations are described along with FIGS. 11A, 11B and 11C.
7. The electronics module 1570 would also compare the value of the activity with a pre-set threshold and turn the LED 1574 red if above the threshold and green if below.
8. The TASSO 1510 with module 1520 are then disposed of. The PLR 1550 will turn off after the module 1480 is removed to be available for another reading.

It is also envisioned that a contact switch (not shown) could be added to the top of the electronics module 1570 that would be activated when the module 1520 is inserted into the electronics module 1570 to automatically turn the electronics on, eliminating the need for the switch 1572. The contact switch may be located at different places with a preferred embodiment requiring that it activate once the reaction zones e.g. 1531 and 1532 align with the photodiodes 1591 and 1592 respectively.

FIG. 16 is a schematic view of an integrated and fully disposable point-of-care TET coupled enzyme assay system 1600 including a TASSO blood collection device 1510 with initiation button 1512. The standard TASSO blood collection vial is replaced by the TET coupled enzyme assay test module 1680 similar to the TET module 1520 of FIG. 15 or the TET module 1400 of FIG. 14. The TET module 1680 includes the test 1672, positive control 1673 and negative control 1671 blood filtration strips similar to the strips 1522, 1523 and 1521 respectively of the module 1520 of FIG. 15 but the module 1680 also includes the cylindrical housing 1690 with photodiodes 1691,1692 and 1693 similar to the cylinder 1580 and photodiodes 1591, 1592 and 1593 of the separate PLR 1550 of FIG. 15. The photodiodes 1691,1692, 1693 and 1694 (not shown) with cables 1695, 1696, 1697 and 1698 (not shown) perform the same function as the photodiodes 1591-1594 and cables 1595-1598 of FIG. 15.

The integrated assay 1600 has an electronics module 1670 attached to the bottom of the upper detection module 1690 into which the wires 1695-1698 bring the signals from the photodiodes 1691-1694 to the electronic circuitry. An embodiment of such electronic circuitry is shown in FIG. 17.

Operation of the system 1600 can be similar to that of the system 1500 of FIG. 15 with the TASSO 1510 button 1512 initiating both the collection of blood and the activation of the electronics module 1670 to measure the luminescence from the reaction zones (hidden) in the TET module 1690. The values produced could be qualitative and/or quantitative measurements of the assay shown by the color on the LED 1674 and numerically on the display 1676 respectively.

The two-piece embodiment is preferred if multiple tests need to be performed where the TASSO/TET 1510/1480 modules are disposable and the PLR 1550 is multi-use. For single assay use such as for concussion at a football game, the fully disposable integrated PoC unit 1600 could be a preferred embodiment.

FIG. 17 is a block diagram of an embodiment of the electronics module 1700 that has features that would be incorporated into either or both electronic module embodiments 1570 of FIG. 15 and 1670 of FIG. 16. The module 1700 has a battery 1770, up to N photodiodes PD1 1701, PD2 1702, PD3 1703 through PDN 1704 whose signal is amplified and/or filtered through the amplifiers 1711, 1712, 1713 through 1714 whose output is digitized by the analog-to-digital converter(s) 1720. The digital signal is sampled into FIFO buffer memory 1730 and input to the central processing unit (CPU) 1740 with the A-to-D converter(s) (ADCs) 1720, First-in, First-out (FIFO) Memory 1730 and CPU 1740 synchronized by the clock/timing sub system 1750.

It is also envisioned that a preferred embodiment could allow the CPU 1740 to read directly from the ADC 1720 instead of through the FIFO memory 1730.

In some embodiments, the CPU 1740 has one or more buttons/switches 1747 such as the start button 1572 of FIG. 15 or may receive input from depression of the TASSO button 1512 of FIGS. 15 and 16. The CPU 1740 also has assay data memory 1741, program memory 1742, and connects to a telemetry sub-system 1760 with Antenna 1765.

The telemetry subsystem 1760 with antenna 1765 may be configured to operate using a standard wireless protocol, for example: Bluetooth, WiFi or Medical Band (MICS). An embodiment of the telemetry sub-system 1760 may also provide a wired connector 1762 (e.g. USB, USB-C, lightning or other) to connect the system 1700 to a local computer, tablet or smart-phone (e.g. iPhone or Android). A wired connector may also be used to recharge the battery 1770.

In embodiments with a separate assay module/card and electronic module such as the configuration shown in FIG. 15, the electronics module may include a bar code reader 1749 to record the serial number of the assay module/card used in the assay that can be transmitted to external equipment using the telemetry sub-system 1760.

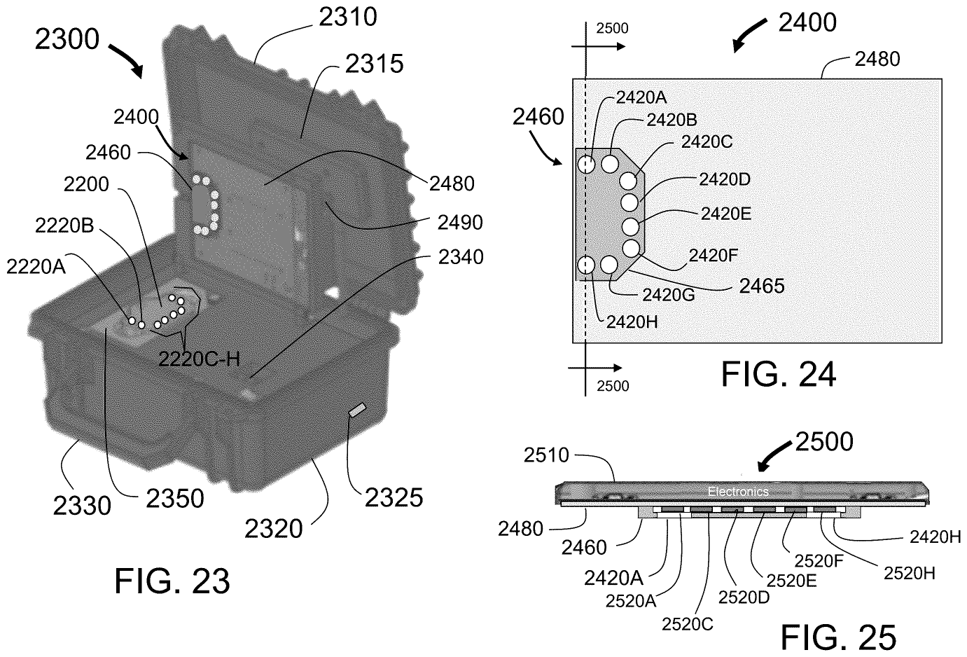

Also connected to the CPU is a temperature sensor 1748 whose reading may be used by the CPU 1740 to adjust parameters in the biomarker detection calculation for a TET assay that may be affected by temperature. It is also envisioned that multiple temperature sensors 1748 may be used to allow measuring the temperature at a number of different locations in the reader 2300 of FIG. 23. Locations can be the outside of the reader case 2300, on the circuit board 2400 of FIG. 23 or on a heating/cooling element 1790.

The output of the detection and measurement calculation(s) in the CPU 1740 can be displayed with the alpha-numeric display 1745 or the LED(s) 1746, or an auditory signal (not shown).

In a preferred embodiment of the electronics module 1700, a heating and cooling element 1790 is included to adjust the system to a pre-set temperature, controlled by the CPU 1740 with input from the temperature sensor 1748. An embodiment of such a device would be a thermoelectric heater/cooler.

Other embodiments would include the GPS transceiver 1772 and the Position sensor (e.g. an accelerometer) 1771 to ensure the device is properly oriented.

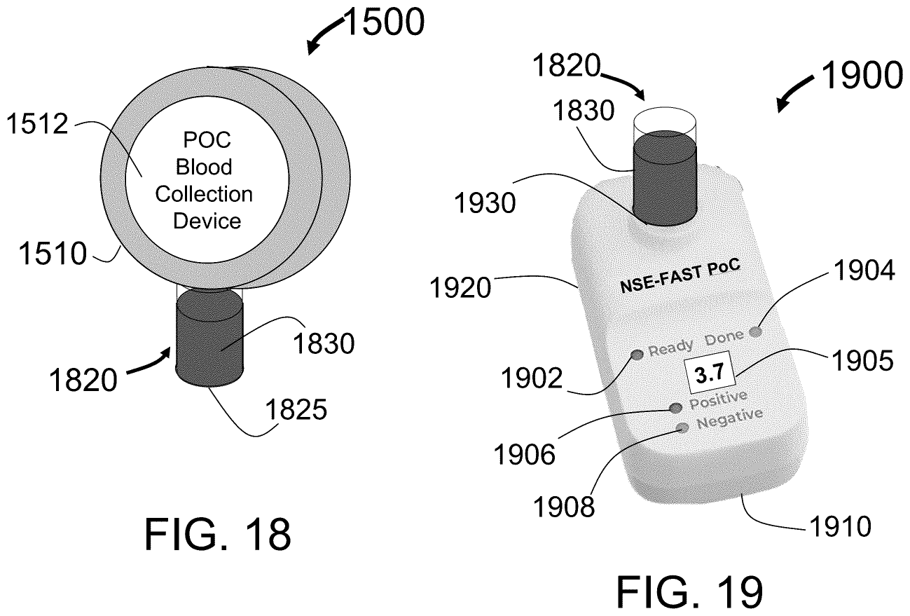

FIG. 18 is a schematic view showing a preferred embodiment of a point of care blood collection device 1500 such as the TASSO® with body 1510 and blood collection activator 1512. A blood collection vial/microtainer 1820 shown with collected blood 1830 and bottom surface 1825 suitable for needle penetration.

In embodiments, the microtainer 1820 has specific materials inside that can be used to pre-treat the blood (for example, the uricase and uric acid shown as A0 101 in FIG. 1). In embodiments, such materials may be freeze died and attached to the inner surface of the microtainer 1820 or simply placed into the microtainer 1820 as a powder or tablet.

FIG. 19 is a schematic view showing a preferred embodiment of a disposable NSE coupled enzyme reaction functional activity stroke test (NSE-FAST) assay 1900. The use of the assay 1900 is similar to that of the Lucira® Covid test sold by Pfizer. The embodiment of the assay 1900 has a case 1920, a numerical display 1905 and four LEDs including a ready LED 1902, a done LED 1904, a positive test LED 1906 and a negative test LED 1908. The assay 1900 has a cylindrical slot 1930 for insertion of the microtainer 1820 with bottom surface 1825 of FIG. 18. At the bottom of the slot 1930 (not shown) is a needle to puncture the bottom surface 1825 of the microtainer 1820 to allow blood 1830 to flow into the assay 1900 where blood separation paper strips similar to the blood separation paper strip set 900 shown in FIG. 12A with negative control strip 911, test strip 912 and positive control strip 913 would be used to separate out plasma that would then flow the into reaction zones similar to those shown in FIGS. 12A, 13A, or 14A. Photo diodes (not shown) similar to the photo diodes 1701 through 1703 of FIG. 17 would detect the luminescence produced in the test, positive control and negative control reaction zone(s) and the CPU 1740 of FIG. 17 would then calculate the activity of the enzyme in the patient sample as previously described. The CPU 1740 would then display the result using the numerical value 1905 and/or the test result positive LED 1906 or test result negative LED 1908. It is also envisioned that the assay 1900 may also utilize embodiments shown in FIG. 13A, 13B or 13C. In embodiments, in addition or instead of the display it is envisioned that the CPU 1740 of FIG. 17 would transmit the result (measurement and/or positive/negative) to external equipment (not shown) through the telemetry sub-system 1760 over the wired connector 1762 or antenna 1765 of FIG. 17.

In a preferred embodiment, an additional LED might indicate an error requiring a new sample to be tested. Alternative embodiments as described above could use auditory signals to indicate when the assay is completed or an error has occurred.

Similar to the Lucira® Covid test, although not shown, the bottom of the case 1920 could have a battery cover 1910 into which one or more batteries (e.g., the battery 1770 of FIG. 17) can be inserted to start the electronics running in preparation for the test. Alternately, the battery could be embedded and the insertion of the microtainer 1820 could turn the system on.

An embodiment of the present invention assay 1900 shown in FIG. 19 is envisioned for tethered enzyme PoC applications for many different biomarkers including the NSE-FA assay and ALT & AST liver enzyme assays shown in FIG. 2.

Also, while the example for FIG. 19 shows use with whole blood from a TASSO device, it is envisioned that the microtainer 1820 can be filled with plasma, serum, urine or liquid into which material from a nasal swab or other material has been suspended or dissolved to facilitate detection of different biomarkers (e.g., viral or bacterial pathogens).

It is envisioned that for assays of liver enzymes ALT and AST the embodiment of the assay 1900 would have separate displays and positive and negative LEDs for each with a preferred embodiment using five blood separation chromatography strips like the strips 1200 of FIG. 12E if the pre-treatment bands 1220 are on the strip or like the strips 900 of FIG. 12A if the pre-treatment materials are in the microtainer 1820.

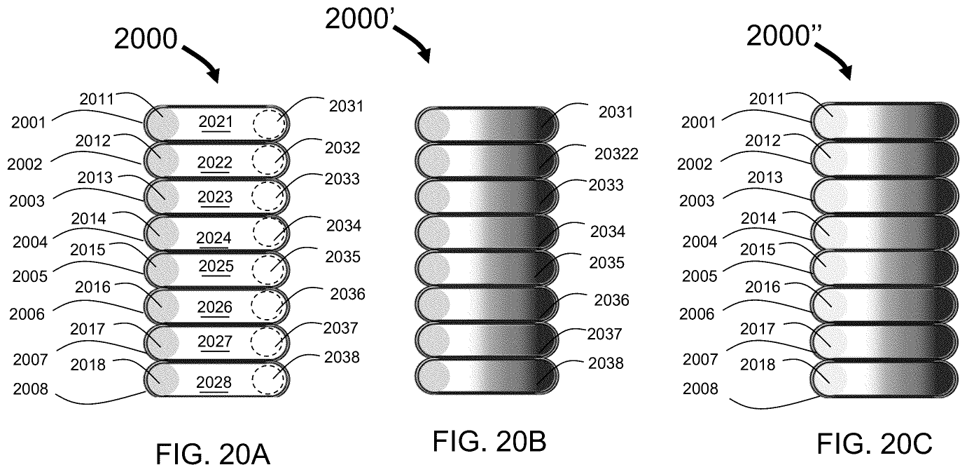

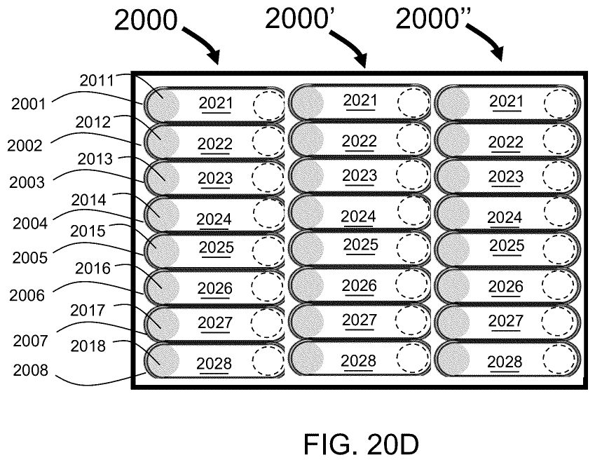

FIGS. 20A, 20B and 20C show a schematic diagram of an embodiment of a present invention Direct Blood Separation (DBS) IVD 2000 comprising 8 connected lateral flow lanes that can function directly from whole blood potentially saving ten minutes or more currently required to centrifuge blood to yield plasma for use in luminescence assays. The DBS IVD 2000 can be designed to fit into modified plate holders that would align the reaction zones within the lanes with the corresponding photodetectors in standard plates (e.g., 96 or 384-well plates), enabling reading by commercial plate readers. Alternatively, these IVDs could be designed to fit with other Photonic Luminescence Readers.

FIG. 20A shows the DBS IVD 2000 with connected lanes 2001 through 2008 having blood separation paper strips 2021 through 2028, luminescence reaction zones 2011 through 2018 and blood input zones 2031 through 2038.

FIG. 20B shows the DBS IVD 2000′ with blood having been deposited in the blood input zones 2031 through 2038 with the blood cells captured on the right side of each lane.

FIG. 20C shows the DBS IVD 2000″ with lanes 2001 through 2008 where plasma has reached the luminescence reaction zones 2011 through 2018 where they will produce luminescence. While the DBS IVD 2000 shown here would include in the reaction zones 2011 through 2018 the necessary components to produce the reaction-based luminescence, it is envisioned that like the lateral flow embodiments shown in FIGS. 12A through 12E, chemical bands used to separate reaction materials or provide pre-treatment of the plasma may be used with the DBS IVD format shown in FIGS. 20A through 20C.

FIG. 20D shows a top view of three of the present invention assays 2000, 2000′ and 2000″, each comprising 8 connected lanes (as shown in FIG. 20A) placed in a 96-well holder that can be inserted into a standard plate reader. For blood testing from multiple patients, this ability to fit 3 such DBS assays in a single plate reader tray is beneficial

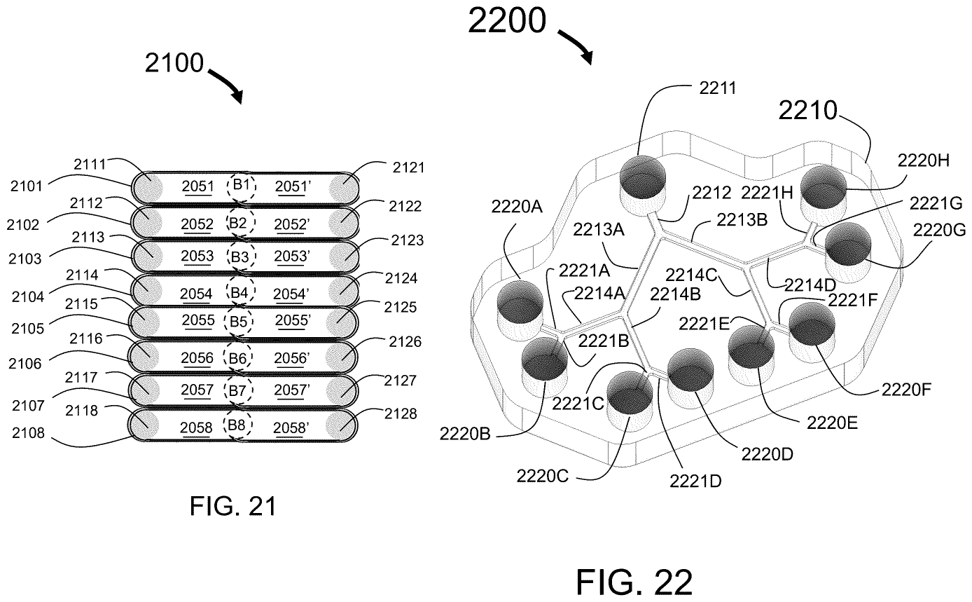

FIG. 21 is a top view of an alternate embodiment of the present invention comprising 8 lanes 2101 through 2108 that can provide 16 assay wells 2111 through 2118 and 2121 through 2128 of IVD measurement from 8 centrally-located input zones B1 through B8 where blood is deposited. The sub-lane 2051 connects the blood input zone B1 to the assay well 2111 and the sub-lane 2051 connects the blood input zone B1 to the assay well 2121. Similarly the sub-lanes 2052 through 2058 connect the blood input zones B2 through B8 to the assay wells 2112 through 2118 respectively. Also, the sub-lanes 2052′ through 2058′ connect the blood input zones B2 through B8 to the assay wells 2122 through 2128 respectively.

Also as previously described, any of these embodiments may include an ADK inhibitor in the blood collection apparatus or in the blood deposit areas such as 2031 through 2038 of FIG. 20A or B1-B8 of FIG. 21; in the zones 2001-2008 of FIG. 20A or 2111-2118 and 2121-2128 of FIG. 21; or in bands not shown.

FIG. 22 is a schematic view of an 8-well assay card 2200 with card body 2210 designed for point-of-care including at-home use. A fluid input port 2211 provides the location for delivery of a fluid sample. The port 2211 is connected to the 8 wells 2220A, 2220B, 2220C, 2220D, 2220E, 2220F, 2220G and 2220H by a series of lateral flow tubes. Specifically, the input port 2211 is connected to the primary tube 2212 connected to secondary tubes 2213A and 2213B. The secondary tube 2213A connects to the two tubes 2214A and 2214B, the secondary tube 2213B connects to the two tubes 2214C and 2214D. The tube 2214A connects to the feeder tube 2221A for the well 2220A as well as the feeder tube 2221B for the well 2220B. The tube 2214B connects to the feeder tube 2221C for the well 2220C and to the feeder tube 2221D for the well 2220D.

The tube 2214C connects to the feeder tube 2221E for the well 2220E and to the feeder tube 2221F for the well 2220F. The tube 2214D connects to the feeder tube 2221G for the well 2220G and to the feeder tube 2221H for the well 2220H.

While the card 2200 is shown as transparent to better show the lateral flow tubes the preferred embodiment would likely be made from a white plastic to better reflect light within the 8 wells 2220A through 2220H and to prevent light from one well reaching the site of another well.

The card 2200 of FIG. 22 is designed to accept a body fluid, for example blood plasma, serum, urine or a liquid in which a nasal or other swab has been soaked.

For whole blood, a preferred embodiment would include sufficient blood separation paper as described for FIGS. 12A through 12E to allow all the blood cells to be captured and plasma to then flow through the lateral flow tubes to the wells 2220A through 2220H.

In a preferred embodiment the wells 2211 and 2220A through 2220H as well as the lateral flow tubes 2213A, 2213B, 2214A, 2214B,2214C, 2214D, 2221A through 2221H would be coated during manufacturing. Examples of suitable coatings include:
poly-ethylene-glycol (PEG),
poly-ethylene-oxide (PEO),
tween or other coatings to prevent inadvertent attachment of proteins to the surface of the wells or tubes.

FIG. 23 is a schematic view of a point-of-care reader 2300 for the 8-well assay card 2200 of FIG. 22. The reader 2300 has a top 2310 that can be opened and closed, bottom case 2320, rechargeable battery 2340 and USBC port 2325, handle 2330, electronics package 2400 with printed circuit board 2480, photo-detector scaffold 2460 and display 2315 (the bottom of the display is shown here; the display itself faces up on the outside of the top 2310).

FIG. 23 shows the assay card 2200 of FIG. 22 with wells 2220A through 2220H after a sample has been introduced into the assay card 2200 and the card 2200 has been placed into the card slot 2350 in the bottom case 2320. After placement the top 2310 is closed placing photo-detector windows 2420A through 2420H of the reader circuit pack 2400 directly over the wells 2220A through 2220H of the assay card 2200. In a preferred embodiment, closing the case enables the photo-detectors (not shown) behind the photo-detector windows 2420A-2420H to begin measuring the light from the wells 2220A through 2220H.

While the display 2315 is shown attached to the case top 2310 with a cable 2490, it is envisioned that it could be instead attached to the top of the printed circuit board 2480 that is attached through the case top 2310. It is also envisioned that with a few LEDs for power etc on the display 2315, the actual user interface could be included as an APP on a cell phone or tablet connected to the CPU 1740 1740 of FIG. 17 through the telemetry sub-system 1760 of FIG. 17 using a protocol such as Bluetooth.

While not shown in FIG. 23, it is envisioned that the reader 2300 would include under the card slot 2350 a heating and cooling element (e.g., a thermoelectric device) with a temperature sensor such as the temperature sensor 1748 and heating and cooling element 1790 of FIG. 17.

FIG. 24 is a bottom view of the electronics package 2400 showing the printed circuit board 2480, photo-detector scaffold 2460 and the eight photo-detector windows 2420A through 2420H. Photo-detector windows will help reduce any crosstalk where the light from one well can be detected by a photo-detector for a different well.

FIG. 25 shows a cross-sectional view at 2500 -2500 of FIG. 24 showing the printed circuit board 2480 with electronic components 2510 such as described for the circuit 1700 of FIG. 17. Shown mounted to the bottom of the printed circuit board 2480 within the photo-detector scaffold 2460 are photo-detectors 2520A, 2520H and 2520C through 2520F. The photo-detectors 2520B and 2520G are hidden behind 2520A and 2520H respectively. Visible in FIG. 25 are the clear photo-detector windows 2420A and 2420H that allow light from the wells 2220A and 2220H of FIG. 22 to reach the photo-detectors 2520A and 2520H respectively. The other 6 widows 2420B through 2420G are hidden in the FIG. 25 cross sectional view.

The scaffold 2460 in a preferred embodiment would be made of a light blocking material and would engage the top of the card 2200 of FIGS. 22 and 23 when the case 2300 is closed. The scaffold 2460 should also be easily cleaned in case any of the fluid in the card 2200 gets onto the surface of the scaffold 2460.

Throughout this specification we describe use of freezing and freeze-drying as important to the present invention as these will prevent premature activation of chemical reactions. It is envisioned that other methods can be used to also prevent premature activation of the chemical reactions described herein and embodiments using these other methods may also facilitate production of the present invention assays. Examples include manipulations of the combination of pressure and temperature, using powdered components including enzymes and the use of reaction inhibitors or dissolvable physical barriers between layers. Another technique envisioned is using encapsulated or caged components to prevent premature mixing of components. A final version is to utilize magnetic particles that can remain separated until the field is removed.

While the well versions of the present invention coupled enzyme assays are intended for use with plate readers capable of measuring luminescence, it is envisioned that small dedicated devices could be implemented to measure the luminescence from the wells using photo-diodes, CCD arrays, photomultiplier tubes or any other photon detection or measurement device.

While the present invention embodiments of the point-of-care assays show the use of photodiodes, embodiments using other photon detection or measurement devices including CCD arrays and photomultiplier tubes are envisioned.

While the present invention specification describes assays for Neuron Specific Enolase, these embodiments are equally appropriate and are applicable to an assay for any active enolase enzyme.

It is also envisioned that embodiments with only one layer for all wells could be produced where the 2^{nd} layer would be in its own separate well and use of the strip would involve pipetting the sample in to a well with the first layer then removing that fluid and introducing it into a second well with components that in other embodiments would have been a second layer.

While the present invention embodiments envision introducing one, two or three layers as liquids to be freeze dried, it is also envisioned that embodiments producing an equivalent of such layers can be accomplished using frozen or pre-dried powders, pills or disks that are produced and then placed into the well.

The paper lateral flow embodiments shown herein could also be produced by having stacked paper to provide the equivalent of layers or bands as described herein.

It is also envisioned that the equivalent of the second layer(s) can be provided by pipetting an additional liquid sample into wells or lateral flow strips before introducing a patient liquid sample.

While the use of tethered enzymes is preferred, embodiments using untethered enzymes would also function for the present invention assays.

Finally, it is envisioned that a single sheet of blood separation paper with pre-set barriers could simplify the design and production of a multiple zone lateral flow assay.

Various other modifications, adaptations, and alternative designs are of course possible in light of the above teachings. Therefore, it should be understood at this time that, within the scope of the appended claims, the invention can be practiced otherwise than as specifically described herein.

What is claimed is:

1. A coupled tethered enzyme luminescence assay adapted to measure the amount of enzymatic activity of neuron specific enolase in a liquid sample derived from the blood of a patient comprising:
at least one test well and at least one positive control well, each comprising a freeze-dried mixture of a plurality of components;
the test well comprising 2-phosphoglycerate, an ADK inhibitor, a plurality of first tethered enzyme nanobots, each of the first tethered enzyme nanobots, each first tethered enzyme nanobot formed by tethering at least one hundred pyruvate kinase enzyme to a silica nanoparticle and a plurality of second tethered nanobots, each second tethered enzyme nanobot formed by tethering at least one hundred luciferase enzyme molecules to a silica nanoparticle, the tethering being structured to provide oriented immobilization of the tethered enzymes pyruvate kinase and luciferase;
each of the pyruvate kinase and luciferase enzymes having two different types of affinity tags comprising a first type of affinity tag adapted to facilitate extraction of enzymes from a liquid and a second type of affinity tag adapted to provide tethering to a silica nanoparticle;
the positive control well, including all the components of the at least one test well with the addition of a preset amount of an enolase enzyme.

2. The assay of claim 1 where the affinity tag to facility extraction from a liquid is a 6xhis tag.

3. The assay of claim 1 where the affinity tag to provide tethering to a silica nanoparticle is an SiO2 tag.

4. The assay of claim 1 where the at least one positive control well comprises two layers introduced separately with the assay being frozen following introduction of the first layer.

5. The assay of claim 4 where the assay is frozen at a temperature below −70 degrees centigrade following introduction of the first layer into the at least positive control well.

6. The assay of claim 4 where the second layer comprises a pre-set amount of an enolase enzyme.

7. The assay of claim 6 where the assay is frozen, and then freeze-dried after introduction of the second layer to the frozen first layer of the at least one positive control well preventing premature reaction between the enolase in the second layer and the 2-phosphoglycerate in the first layer of the at least one positive control well.

8. The assay of claim 7 where the freeze-dried assay is sealed into an air-tight package in the presence of an inert gas to prevent moisture from causing premature reaction between the enolase in the second layer and the 2-phosphoglycerate in the first layer of the at least one positive control well.

9. The assay of claim 8 where the inert gas is nitrogen.

10. The assay of claim 8 where the packaging occurs in the presence of the inert gas at a pressure of less than 1 atmosphere.

11. The assay of claim 1 further including at least one well adapted to produce luminescence allowing measurement of the level of hemolysis in the liquid sample.

12. A coupled tethered enzyme luminescence assay adapted to measure the amount of enzymatic activity of neuron specific enolase in a liquid sample derived from the blood of a patient comprising:
at least one test well and at least one positive control well, each comprising a freeze-dried mixture of a plurality of components, the at least one positive control well including a first layer and a second layer separately introduced into each positive control well during assay preparation, the test well including only a single layer having the same plurality of components as the first layer of the at least one positive control well;
the first layer of the positive control well comprising 2-phosphoglycerate, an ADK inhibitor, a plurality of first tethered enzyme nanobots, each first tethered enzyme nanobot formed by tethering at least one hundred pyruvate kinase enzyme to a silica nanoparticle and a plurality of second tethered nanobots, each second tethered enzyme nanobot formed by tethering at least one hundred luciferase enzyme molecules to a silica nanoparticle, the tethering being structured to provide oriented immobilization of the tethered enzymes pyruvate kinase and luciferase;
each of the pyruvate kinase and luciferase enzymes having two different types of affinity tags comprising a first type of affinity tag adapted to facilitate extraction of enzymes from a liquid and a second type of affinity tag adapted to provide tethering to a silica nanoparticle; the second layer of the at least one positive control well including a preset amount of an enolase enzyme.

13. The assay of claim 12 where the affinity tag to facility extraction from a liquid is a 6xhis tag.

14. The assay of claim 12 where the affinity tag to provide tethering to a silica nanoparticle is an SiO2 tag.

15. The assay of claim 12 where the assay is frozen following introduction of the first layer into the at least one positive control well.

16. The assay of claim 15 where the assay if frozen at a temperature below −70 degrees centigrade.

17. The assay of claim 15 where the assay is frozen, and then freeze-dried after introduction of the second layer to the frozen first layer of the at least one positive control well preventing premature reaction between the enolase in the second layer and the 2-phosphoglycerate in the first layer of the at least one positive control well.

18. The assay of claim 17 where the freeze-dried assay is sealed into an air-tight package in the presence of an inert gas to prevent moisture from causing premature reaction between the enolase in the second layer and the 2-phosphoglycerate in the first layer of the at least one positive control well.

19. The assay of claim 18 where the inert gas is nitrogen.

20. The assay of claim 18 where the packaging occurs in the presence of the inert gas at a pressure of less than 1 atmosphere.

21. The assay of claim 12 further comprising a reader adapted to measure the luminescence produced when a liquid sample comprising enzymatically active neuron specific enolase is introduced into the assay, the reader further adapted to calculate the amount of enzymatic activity of the neuron specific enolase in the liquid sample by comparison of the measured luminescence produced by the Luciferase enzymes in the second tethered nanobots of the at least one positive control well and at least one test well.

22. The assay of claim 21 further including at least one well adapted to produce luminescence allowing measurement of the level of hemolysis in the liquid sample.

23. A coupled tethered enzyme luminescence assay adapted to measure the amount of enzymatic activity of neuron specific enolase in a liquid sample derived from the blood of a patient comprising:
at least one test well and at least one positive control well, each comprising a freeze-dried mixture of a plurality of components;
the test well comprising 2-phosphoglycerate, an ADK inhibitor, a plurality of first tethered enzyme nanobots, each of the first tethered enzyme nanobots, each first tethered enzyme nanobot formed by tethering at least one hundred pyruvate kinase enzyme to a silica nanoparticle and a plurality of second tethered nanobots, each second tethered enzyme nanobot formed by tethering at least one hundred luciferase enzyme molecules to a silica nanoparticle, the tethering being structured to provide oriented immobilization of the tethered enzymes pyruvate kinase and luciferase;
each of the pyruvate kinase and luciferase enzymes having two different types of affinity tags comprising a first type of affinity tag being a 6xhis tag adapted to facilitate extraction of enzymes from a liquid and a second type of affinity tag being an SiO2 tag adapted to provide tethering to a silica nanoparticle;
the positive control well, including all the components of the at least one test well with the addition of a preset amount of an enolase enzyme.

24. The assay of claim 23 where the at least one positive control well comprises two layers introduced separately with the assay being frozen following introduction of the first layer.

25. The assay of claim 24 where the assay is frozen at a temperature below −70 degrees centigrade following introduction of the first layer into the at least one positive control well.

26. The assay of claim 24 where the second layer comprises a pre-set amount of an enolase enzyme.

27. The assay of claim 26 where the assay is frozen, and then freeze-dried after introduction of the second layer to the frozen first layer of the at least one positive control well preventing premature reaction between the enolase in the second layer and the 2-phosphoglycerate in the first layer of the at least one positive control well.

28. The assay of claim 26 where the freeze-dried assay is sealed into an air-tight package in the presence of an inert gas to prevent moisture from causing premature reaction between the enolase in the second layer and the 2-phosphoglycerate in the first layer of the at least one positive control well.

29. The assay of claim 28 where the inert gas is nitrogen.

30. The assay of claim 28 where the packaging occurs in the presence of the inert gas at a pressure of less than 1 atmosphere.

31. The assay of claim 23 further comprising a reader adapted to measure the luminescence produced when a liquid sample comprising enzymatically active neuron specific enolase is introduced into the assay, the reader further adapted to calculate the amount of enzymatic activity of the neuron specific enolase in the liquid sample by comparison of the measured luminescence produced by the Luciferase enzymes in the second tethered nanobots of the at least one positive control well and at least one test well.

32. The assay of claim 23 further including at least one well adapted to produce luminescence allowing measurement of the level of hemolysis in the liquid sample.
